SEQUENCE LISTING (56 sequences)

SEQ ID NO: 1 - protein, 510 aa, Vibrio vulnificus
GDKTKVVVDLAQIFTVQELKERAKVFAKPIGASYQGILDQLDLVHQAKGRDQIAASFELNKKINDYIAEHPTSGRNQALTQLKEQVTSALFIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSAEMILLKKFADQSYLSQLDSDRMDQIEGIYRSSHETDIDAWDRRYSGTGYDELTNKLASATGVDEQLAVLLDDRKGLLIGEVHGSDVNGLRFVNEQMDALKKQGVTVIGLEHLRSDLAQPLIDRYLATGVMSSELSAMLKTKHLDVTLFENARANGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKGSL

SEQ ID NO: 2 - protein, 186 aa, Vibrio vulnificus
FIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSAEMILLKKFADQSYLSQLDSDRMDQIEGIYRSSHET

SEQ ID NO: 3 - protein, 510 aa, Vibrio harveyi
GDKTKVVVDLAQIFTVQELKERAKVFAKPIGASYQGILDQLDLVHQAKGRDQIAASFELNKKINDYIAEHPTSGRNQALTQLKEQVTSALFIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSVEMILLKKFADQSYLSQLDSDRMDQIEGIYRSSHETDIDAWDRRYSGTGYDELTNKLASATGVDEQLAVLLDDRKGLLIGEVHGSDVNGLRFVNEQMDALKKQGVTVIGLEHLRSDLAQPLIDRYLATGVMSSELSAMLKTKHLDVTLFENARANGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKDSL

SEQ ID NO: 4 - protein, 186 aa, Vibrio harveyi
FIGKMQVAQAGIDAIAQTRPELAARIFMVAIEEANGKHVGLTDMMVRWANEDPYLAPKHGYKGETPSDLGFDAKYHVDLGEHYADFKQWLETSQSNGLLSKATLDESTKTVHLGYSYQELQDLTGAESVQMAFYFLKEAAKKADPISGDSVEMILLKKFADQSYLSQLDSDRMDQIEGIYRSSHET

SEQ ID NO: 5 - protein, 510 aa, Vibrio ordalii
GDKTKVVVDLAQIFTVQELKERAKVFAKPIGASYQGILDQLDLVHQAKGRDQIAASFELNKKINAYIAEHPTSGRNQALTQLKEQVTSALFIGKMQIAQAGIDAIAQTRPELAARIFMVAIEEANGKHVGLTDMMVRWANEDPYLAPKHGYKGETPSDLGFDAKYHVDLSEHYADFKQWLETSQSNGLLSKAMLDESTKTVHLGYSYQELQDLTGVESVQMAFYFLKEAAKKADPISGDSAEMILLKKFADQSYLSQLDSDRMDQIEGIYRSSHETDVDAWDRRYSGKGYDELTNKLASATGVDEQLSVLLDDRKGLLIGEVHGSDVNGLRFVNEQMDALKKQGVTVIGLEHLRSDLAQPLIDRYLATGVMSSELSAMLKTKHLDVALFENARAHGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKDSL

SEQ ID NO: 6 - protein, 186 aa, Vibrio ordalii
FIGKMQIAQAGIDAIAQTRPELAARIFMVAIEEANGKHVGLTDMMVRWANEDPYLAPKHGYKGETPSDLGFDAKYHVDLSEHYADFKQWLETSQSNGLLSKAMLDESTKTVHLGYSYQELQDLTGVESVQMAFYFLKEAAKKADPISGDSAEMILLKKFADQSYLSQLDSDRMDQIEGIYRSSHET

SEQ ID NO: 7 - protein, 511 aa, Vibrio cholerae
SGNKAKVAVDLAQIFTVQELKERAKVFAKPIGASYQGILDQLDLVHQAKGRYQIAASFELNKKINDYIAEHPTSGRNQALTQLKEQVTSALFIGKMQVAQAGIDAIAQTRPELATRIFMVAIEEANGKHVGLTDMMLRWANEDPYLAPKHGYKGEMPSDLGFDAKYHVDLGEHYADFKQWLETSQSNGLLSKATLDESTKTVHLGYSYQELQDLTGAESVQMAFYFLKEAAKKADPISGDSAEMILLKKFADQNYLSQLDSDRMDQIEGIYRSSHETDVDAWDRRYSGKGYDELTNMLASATGVDEQLSVLLDDRKGLLIGEVHGSDVNGLRFVNEQMEALKKQGVTVIGLEHLRSDLAQPLIDRYLATGVMSSELSAMLKTKHLDVTLFENARVNGMRIVALDANSSARPNVQGTEHGLMYRAGAANNIAVEVLQNLPDGEKFVAIYGKAHLQSHKGIEGFVPGITHRLDLPALKVSDSNQFTVEQDDVSLRVVYDDVANKPKITFKGSL

SEQ ID NO: 8 - protein, 186 aa, Vibrio cholerae
FIGKMQVAQAGIDAIAQTRPELATRIFMVAIEEANGKHVGLTDMMLRWANEDPYLAPKHGYKGEMPSDLGFDAKYHVDLGEHYADFKQWLETSQSNGLLSKATLDESTKTVHLGYSYQELQDLTGAESVQMAFYFLKEAAKKADPISGDSAEMILLKKFADQNYLSQLDSDRMDQIEGIYRSSHET

SEQ ID NO: 9 - protein, 511 aa, Vibrio splendidus
GNKTKLVVDLSTIMTKQELKDGGKVFAKPIGASYQAILDQVELVHSSIGRDQVGASFELNKQINNYLAEHPTSGRNLALTTLKEQVNTALFSGKMKVTQESIDAIAQTRTDLAARIYVVAMEEANGEHVGLTDMMVRWANEDPYLSPKQGYAGETPSDLGFDAKYHIELGEQYSDFKLWLEKSQSADLLSKAALDEATKTVHLGYSYQELQDLTGVESVQMAFYFLKEAAKKSDSTTSDSAEMILLKKFADQGYISQLETDRMDHIEGIYRSSHETDVDNWDRRYSGAGYDELSDKLAGANGGVEEQLSVLLNERKGLLIGEVHGSDVNGLRFVNEQMDALKKQGVTVIGLEHLRSDLAQPLIDNYLSTGIMSSELSAMIKTKHLDITLFENARANGMRILALDANSTARPTVQGTEHGLMYRAGAANNVAVDALQALPDGEKFVAIYGKAHLQSHKGIESFVPGITHRLGLPALKVSASDQFVIEQDDKTLRTVYDDVANKPKIEFRASL

SEQ ID NO: 10 - protein, 186 aa, Vibrio splendidus
FSGKMKVTQESIDAIAQTRTDLAARIYVVAMEEANGEHVGLTDMMVRWANEDPYLSPKQGYAGETPSDLGFDAKYHIELGEQYSDFKLWLEKSQSADLLSKAALDEATKTVHLGYSYQELQDLTGVESVQMAFYFLKEAAKKSDSTTSDSAEMILLKKFADQGYISQLETDRMDHIEGIYRSSHET

SEQ ID NO: 11 - protein, 510 aa, Moritella dasanensis
GNKAKQSADLSEVFTKDQLKKNAKVFAKPIGVSYQRILDQVGLVHSTTGRDQIAASFELNKQIDAYVEANPASGRNQAFNQLKGQITNALFNGDIQVAKEGISEIAQTRPELAARIYIIAQEEANGKNLGLTDLMVRWAKEDPYLSAKNGYQGDIPSDLGFEAKFHVELGSQYADFKQTLEKAQVEGLLTKAVIDESTKTVHLGYTYQELQDQTGTESVQMAAYFLKEAAKKSDPTSADSAEMILLNKFADKNYITELERQRIDQIESIYRSSHDTDIAGWDKRYSGTALNELNSQLGAATSVEAQLALLLEKRNGLLIGESHGSDVNGLRFVNEQMDALKAQGVSVIGLEHLRADLAQPLIDSYLSSGDMSSELRIMLKTKHLDISLFENARAKGLRIVALDANSTTRPTIQGTEHGLMYRAGAANNVAVETLSGLPAGEKFVAIYGNAHLQSHKGIEGFVPGITHRLDLPGLKISETNQFKAQADDLSQRVIYGDVLNKAKIEFTNSL

SEQ ID NO: 12 - protein, 186 aa, Moritella dasanensis
FNGDIQVAKEGISEIAQTRPELAARIYIIAQEEANGKNLGLTDLMVRWAKEDPYLSAKNGYQGDIPSDLGFEAKFHVELGSQYADFKQTLEKAQVEGLLTKAVIDESTKTVHLGYTYQELQDQTGTESVQMAAYFLKEAAKKSDPTSADSAEMILLNKFADKNYITELERQRIDQIESIYRSSHDT

SEQ ID NO: 13 - protein, 509 aa, Aeromonas salmonicida
PGKVVAQERASSLFADAYSPDELKKAAQVFAKPIGESYQGILDQLAVLHGATGQVQVEAALRLNKLIDDYQARHEGSGRNPALSKLQSQLNGGLYHGELASLQADVAALAKSRPDLAALVIGKAAEEAKGQHPGLTQMLLRWAAQDPYLAAKGGYQGQAPADLPFDASFHVVLGEQYGELKRWLADAQSKGLLSKAVLDETGKVLHLGYSYQELQDMTGDQSAQMTVYFIKEAAKQAAPGSELSAEMIMLDKFADRRYLGELGSRRLEQVESIYRSSKQTDVAAWDARYAGNALRDLNDQVAQESTLAGQLSRLLENRNGLLIGETHGSDVNGLRFVNEQMDVLKAQGVTVIGLEHLRGELAQPLIDRYLAGGDMSPELATMLKTKHLDPSLFERAREKGLRIVALDDGSTARPAIAGTEHGLMYRAGAANNVAVDVLGKLPAGEKFVAIYGSAHLASHKGIEGFVPGITHRLGLPALKVDADNRFRLQEDDTSQRVEYGDVARKWTPL

SEQ ID NO: 14 - protein, 186 aa, Aeromonas salmonicida
YHGELASLQADVAALAKSRPDLAALVIGKAAEEAKGQHPGLTQMLLRWAAQDPYLAAKGGYQGQAPADLPFDASFHVVLGEQYGELKRWLADAQSKGLLSKAVLDETGKVLHLGYSYQELQDMTGDQSAQMTVYFIKEAAKQAAPGSELSAEMIMLDKFADRRYLGELGSRRLEQVESIYRSSKQT

SEQ ID NO: 15 - protein, 512 aa, Aeromonas hydrophila
PGKTGVTERTARLFADVYSPDELKKAAQVFAKPIGESYQQILDQLATLHGASGQAKVEAALRLNNLIDDYLVKHEGSGRNPALSKLQSQLHGNLYRGELASLQAEVTALAKTRPDLAAIVIGKAAEEAQGQHPGLTQMVLRWAAQDPYLAAKAGYQGVVPADLPFDARFHIALGEQHDDLKKWLTEAQGKGLLNRAVLDDTRKVLHLGYSYQELQDMTGEQSAQMAVYFIKEAAKQAAPGSELSAELIMLDKFGDRRYLGELESRRIAQIENIYHSSKQTDVAAWDARYGGDALRTLNNQLDGESTLAGQLSRLLDNRNGLLIGETHGSDVNGLRFVNEQMDALKIQGVTVIALEHLRSELAQPLIDRYLAGGEMSPELTSMLKNKHLEPSLFERARERGMRIVALDDGSTARPAIAGTEHGLMYRAGAANNVAVEVLGKLPAGEKFVAIYGSAHLGSHKGIEGFVPGITHRLGLPALKVDADNRFHLQADDVSQRVEYADVGRKWTPVAAL

SEQ ID NO: 16 - protein, 186 aa, Aeromonas hydrophila
YRGELASLQAEVTALAKTRPDLAAIVIGKAAEEAQGQHPGLTQMVLRWAAQDPYLAAKAGYQGVVPADLPFDARFHIALGEQHDDLKKWLTEAQGKGLLNRAVLDDTRKVLHLGYSYQELQDMTGEQSAQMAVYFIKEAAKQAAPGSELSAELIMLDKFGDRRYLGELESRRIAQIENIYHSSKQT

SEQ ID NO: 17 - protein, 561 aa, Photorhabdus temperate
MEYEYDKTDDRKRKHSTQWADYEEKSFVPTLDLSQSRQHNPSHDALNRADNHETSPLLHNLITSDNLRKEAAVFAKRIGSSYQGILDGLHRIHTLSGNEQLTAGFELHQRITRYLKTHPDSKRNTSLRRMQTQLEDLMFTGTLQMVRSPLLEMAETRPDMASRIYQIACNETRGNTPGLTDLMVRWVKEDPYLATKTGYQGEIPNDLPFDPKFHVELGAQFDDFKKWLNIAQSQGLLTHARLDEPSKRVHLGYSYNELLDMTGVESVQMAVYFLKEAAKQADPGFAGSQEAILLNRFANPAYLAQLEQGRLSQIEAIYHSSHNTDVAAWDKQFDADALVQLNHQLNGSTDLDSQLSLLLKNRQGLLIGESHGSDLNGLRFVNEQMNALKAHGVSVIGLEHLRSDLAQPLIDNFLASGDMSAELAAMIKTKHLDPALFEQARIKSMKIIALDDNSTTRPVVAGTQHGLMYRAGAANNVAVERLQQLPVGEKFVAIYGNAHLQSHEGIDHFIPGMTHRLGLPALKVDANNHFVAQADDTSQRKRYDDVANVPRIQLIPQAKLL

SEQ ID NO: 18 - protein, 220 aa, Photorhabdus temperate
FELHQRITRYLKTHPDSKRNTSLRRMQTQLEDLMFTGTLQMVRSPLLEMAETRPDMASRIYQIACNETRGNTPGLTDLMVRWVKEDPYLATKTGYQGEIPNDLPFDPKFHVELGAQFDDFKKWLNIAQSQGLLTHARLDEPSKRVHLGYSYNELLDMTGVESVQMAVYFLKEAAKQADPGFAGSQEAILLNRFANPAYLAQLEQGRLSQIEAIYHSSHNT

SEQ ID NO: 19 - protein, 531 aa, Xenorhabdus nematophila
ALSGKNKTLETVIAENDGTPSLNELITKDGLRKKASVFAKPIGPAYQAILDKLDHIHNLTGNEQLSAGFELYQRITRYLNEHPDSKRNTALSGVQTQLGDIMFRGALQEVRSPLLEIAQTRPEMASRIYQIARNEARGDTPGLTDLMVRWVKEDPYLAAKLGYQGEIPADLAFNPKFHVDLGDQFDDFKQCLSKAQDKGLLINARIDEQNKRVHLGYSYNELLDMTGSEDVKMAVYFLKEVAKQADPNFAGSHEAILLNRFANPAYLVQLEQGRLAQIEAIYHSSHQTDIAAWDKQYSSDALTQLNRQLSDGTDLNSQLSLLLKDRQGLLIGESHGSDLNGLRFVNEQMDALKVHGVTVIGLEHLRSDLAQPLIDKFLAGGDMPAELTAMIETKHLPVDLFEQAKSKGIKIIALDDNSTTRPAIEGSQHGLMYRAGAANNVAVKRLGLLAEGEKFVAIYGDAHLQSHEGIDHFVPGMTHRLGLPALKVDANNRFTAQADDISLRKHYDDVPQLEKNLYKPNRVVGGDLEVL

SEQ ID NO: 20 - protein, 186 aa, Xenorhabdus nematophila
FRGALQEVRSPLLEIAQTRPEMASRIYQIARNEARGDTPGLTDLMVRWVKEDPYLAAKLGYQGEIPADLAFNPKFHVDLGDQFDDFKQCLSKAQDKGLLINARIDEQNKRVHLGYSYNELLDMTGSEDVKMAVYFLKEVAKQADPNFAGSHEAILLNRFANPAYLVQLEQGRLAQIEAIYHSSHQT

SEQ ID NO: 21 - protein, 568 aa, Photorhabdus luminescens
MTGVSECSQQRSNLKDDGYISSRKLTGDNMVYEYDKTIERRRNPSIQLNNNEKSSEQALELSQNNPLLHDLITSNNLRKEAAVFAKRIGPSYQEILDELEHLHHLSGNEQLAAGFELHRRITHYLEEHPDSKRNTALRRTQTQFGDLMFTGTLQKIRHSLLEMAETRPEMASHIYQIAREEVKGNTPGLTDLMVRWVKEDPYLAAKTGYQGKIPNDLPFEPKFHVELGAQFDDFKKWLDTAQSKELLTHTRLDEQNKQVHLGYSYNELLDMTGVESVQMAVYFLKEAAKQAEPGSTKSQEDILLHRFANPTYLAQLEHSRLAQIEAIYHSSHDTDVTAWDQQFASDALTQFNHQLNNTVDLNSQLSLLLKDRQGLLIGESHGSDLNGLRFVEEQMEVLKAHGVTVIGLEHLRSDLAQPLIDKFLASGNEPMPAELAALLKTKHLSANLFEQARSKQMKIIALDNNSTTRPTVEGTQHGLMYRAGAANNVAVERLRQLPAGEKFVAIYGNAHLQSHEGIDHFLPGITHRLGLPALKVDENNRFTAQVDNINQRKRYDDVVELPRIQLTS

SEQ ID NO: 22 - protein, 220 aa, Photorhabdus luminescens
FELHRRITHYLEEHPDSKRNTALRRTQTQFGDLMFTGTLQKIRHSLLEMAETRPEMASHIYQIAREEVKGNTPGLTDLMVRWVKEDPYLAAKTGYQGKIPNDLPFEPKFHVELGAQFDDFKKWLDTAQSKELLTHTRLDEQNKQVHLGYSYNELLDMTGVESVQMAVYFLKEAAKQAEPGSTKSQEDILLHRFANPTYLAQLEHSRLAQIEAIYHSSHDT

SEQ ID NO: 23 - protein, 542 aa, Photorhabdus asymbiotica
MVFEHDKTVERKRKPSIQLGNDKEKSSEQALELPQSKQNNPLLHDLITSNNLRKEAAVFAKQIGPSYQGILDGLEHLHNLSGNEQLTAGFELHRRITRYLEEHPDSKRNAALRRTQTQLGDLMFTGTLQEVRHPLLEMAETRPAMASQIYQIARDEAKGNTPGLTDLMVRWVKEDPYLAAKSGYQGKIPNDLPFEPKFHVELGDQFGEFKTWLDTAQNQGLLTHTRLDEQNKQVHLGYSYNELLDMTGGVESVKMAVYFLKEAAKQAEPGSAKSQEAILLNRFANPAYLTQLEQGRLAQMEAIYHSSHNTDVAAWDQQFSPDALTQFNHQLDNSVDLNSQLSFLLKDRQGLLIGESHGSDLNGLRFVEEQMDALKAHGVTVIGLEHLRSDLAQPLIDKFLTSENEPMPAELAAMLKTKHLSVNLFEQARSKQMKIIALDNNSTTRPAEGEHSLMYRAGAANNVAVERLQQLPAEEKFVAIYGNAHLQSHEGIDHFLPGITHRLGLPALKVDENNRFTAQADNINQRKCYDDVVEVSRIQLTS

SEQ ID NO: 24 - protein, 221 aa, Photorhabdus asymbiotica
FELHRRITRYLEEHPDSKRNAALRRTQTQLGDLMFTGTLQEVRHPLLEMAETRPAMASQIYQIARDEAKGNTPGLTDLMVRWVKEDPYLAAKSGYQGKIPNDLPFEPKFHVELGDQFGEFKTWLDTAQNQGLLTHTRLDEQNKQVHLGYSYNELLDMTGGVESVKMAVYFLKEAAKQAEPGSAKSQEAILLNRFANPAYLTQLEQGRLAQMEAIYHSSHNT

SEQ ID NO: 25 - protein, 504 aa, Yersinia kristensenii
IESNVIISKDELKKQASVMGKPIGYSYKKILNIIDLINSTSNSERIKNIFILKSEIERYINEHPSSGRNKAFLTLGEKIDKSLFNSKMQPAKNAILRLSKTQPEMAARLYEVAARESQGSHVGLTNMMHVWISEDGYLTLLKGFEGKIPDRNLLNFDPTYHIATGDQFDECKTKLLQAQSNGELRQVYINESTRSFTIGYTYEEMASFRARGSENSQFFSYILNEVAGRNNSTDRSKELNWLDNCADKKFLKQLQLSRLDQIESIYQRNNKIDFASWDSKYSGISRDRINRELNQYGDVDGQLSVLLRGNQGLLIGETHGSQEEGRRFIIEQISELKRHGVTTIGLEHLRRDHIQPLIDDYYRTGVISPDLNTFLTAKGVKNIVTTAFENKVKIIFLDDNSTSKGSGNHSLMYRAGSANNIAVDILKQIPANEKFVVIYGEAHLKSHIGIESPVSGISHQMKLPILQVDANNRLTVSADDPTQRTIYPRNNTTGSPRIIFPATL

SEQ ID NO: 26 - protein, 189 aa, Yersinia kristensenii
FNSKMQPAKNAILRLSKTQPEMAARLYEVAARESQGSHVGLTNMMHVWISEDGYLTLLKGFEGKIPDRNLLNFDPTYHIATGDQFDECKTKLLQAQSNGELRQVYINESTRSFTIGYTYEEMASFRARGSENSQFFSYILNEVAGRNNSTDRSKELNWLDNCADKKFLKQLQLSRLDQIESIYQRNNKI

SEQ ID NO: 27 - protein, 503 aa, Pasteurella multocida
IGLEGVWTPEVLKARASVIGKPIGESYKRILAKLQRIHNSNILDERQGLMHELMELIDLYEESQPSSERLNAFRELRTQLEKALYLPEMEALKKQILQIPNKGSGAARFLLRTAMNEMAGKTSESTADLIRFALQDTVISAPFRGYAGAIPEAIDFPVKYVIEDISVFDKIQTNYWELPAYESWNEGSNSALLPGLLRESQSKGMLSKCRIIENSLYIGHSYEEMFYSISPYSNQVGGPYELYPFTFFSMLQEVQGDLGFEQAFATRNYFNTLVSDRLSLMENTMLLTESFDYTPWDAIYGDINYDEQFAAMSINERIEKCMNTYRGVAFQNSSKSIDFFLNNLTTFIDNGLTEIAISDLPYDIVQQEISQFLQGSNEWKTLDAMLFNLDKGDINGAFRKLLQSAKDNNIKFRAIGHSDNSVPPFNNPYKSLYYKGNIIAEAIEKLDREGQKFVVFADSSLLNSTPGTGRPMPGLVQYLKIPATVVDSDGAWQFLPDVASSRV

SEQ ID NO: 28 - protein, 207 aa, Pasteurella multocida
YLPEMEALKKQILQIPNKGSGAARFLLRTAMNEMAGKTSESTADLIRFALQDTVISAPFRGYAGAIPEAIDFPVKYVIEDISVFDKIQTNYWELPAYESWNEGSNSALLPGLLRESQSKGMLSKCRIIENSLYIGHSYEEMFYSISPYSNQVGGPYELYPFTFFSMLQEVQGDLGFEQAFATRNYFNTLVSDRLSLMENTMLLTESF

SEQ ID NO: 29 - protein, 9 aa, Influenza virus
YPYDVPDYA

SEQ ID NO: 30 - DNA, 33 bp, Artificial
  Forward primer for amplying rtxA1 gene of Vibrio vulnificus
aaggtaccgtttatcggtaagatgcaagttgcc

SEQ ID NO: 31 - DNA, 32 bp, Artificial
  Reverse primer for amplying rtxA1 gene of Vibrio vulnificus
agaattctcacaaactgcccttgaacgtgatc

SEQ ID NO: 32 - DNA, 30 bp, Artificial
  Forward primer for amplying rtxA1 gene of Vibrio vulnificus
aaggtaccgggtgataaaaccaaggtcgtg

SEQ ID NO: 33 - DNA, 30 bp, Artificial
  Reverse primer for amplying rtxA1 gene of Vibrio vulnificus
aaggtaccggatattgacgcttgggatcgt

SEQ ID NO: 34 - DNA, 169 bp, Artificial
  Coding sequence for mutant form of Vibrio vulnificus DUF5 polypeptide having D3721R/R3841D mutations
atctttatggtcgcgattgaagaagccaacggtaaacacgtaggtttgacggacatgatggttcgttgggccaatgaagaaccatacttggcaccgaagcatggttacaaaggcgaaacgccaagtgaccttggttttgatgcgaagtaccacgtagatctaggtgagc

SEQ ID NO: 35 - DNA, 43 bp, Artificial
  Forward primer for amplifying coding sequence of Vibrio vulnificus DUF5
tacttccaatccaatgctcaagagctgaaagaaagagcaaaag

SEQ ID NO: 36 - DNA, 43 bp, Artificial; identical to SEQ ID NO: 35
  Reverse primer for amplifying coding sequence of Vibrio vulnificus DUF5

SEQ ID NO: 37 - DNA, 43 bp, Artificial; identical to SEQ ID NO: 35
  Forward primer for amplifying coding sequence for DUF5 polypeptide of Vibrio vulnificus

SEQ ID NO: 38 - DNA, 38 bp, Artificial
  Reverse primer for amplifying coding sequence for DUF5 polypeptide of Vibrio vulnificus
ttatccacttccaatgctacaaactgcccttgaacgtg

SEQ ID NO: 39 - DNA, 39 bp, Artificial
  Forward primer for amplifying coding sequence for DUF5 polypeptide of Aeromonas hydrophila
tacttccaatccaatgctccgggcaaaacggtggtgacg

SEQ ID NO: 40 - DNA, 41 bp, Artificial
  Reverse primer for amplifying coding sequence for DUF5 polypeptide of Aeromonas hydrophila
ttatccacttccaatgctagacatcggcgtactcgacccgc

SEQ ID NO: 41 - DNA, 42 bp, Artificial
  Forward primer for amplifying coding sequence for DUF5 polypeptide of Photorabdus asymbiotica
tacttccaatccaatgctccattactccatgacctcatcacc

SEQ ID NO: 42 - DNA, 40 bp, Artificial
  Reverse primer for amplifying coding sequence for DUF5 polypeptide of Photorabdus asymbiotica
ttatccacttccaatgctacacatcatcataacacttgcg

SEQ ID NO: 43 - DNA, 53 bp, Artificial
  Forward primer for amplifying coding sequence of human KRAS
tacttccaatccaatgctatgactgaatataaacttgtggtagttggagctgg

SEQ ID NO: 44 - DNA, 56 bp, Artificial
  Reverse primer for amplifying coding sequence of human KRAS
ttatccacttccaatgctacataattacacactttgtctttgacttctttttcttc

SEQ ID NO: 45 - DNA, 45 bp, Artificial
  Forward primer for amplifying coding sequence of human HRAS
tacttccaatccaatgctatgacggaatataagctggtggtggtg

SEQ ID NO: 46 - DNA, 41 bp, Artificial
  Reverse primer for amplifying coding sequence of human HRAS
ttatccacttccaatgctaggagagcacacacttgcagctc

SEQ ID NO: 47 - DNA, 43 bp, Artificial
  Forward primer for amplifying coding sequence of human NRAS
tacttccaatccaatgctatgactgagtacaaactggtggtgg

SEQ ID NO: 48 - DNA, 41 bp, Artificial
  Reverse primer for amplifying coding sequence of human NRAS
ttatccacttccaatgctacatcaccacacatggcaatccc

SEQ ID NO: 49 - DNA, 40 bp, Artificial
  Forward primer for amplifying human EGF
gcttcgaattctgcacccgggtggtctggttccgcgtgga

SEQ ID NO: 50 - DNA, 41 bp, Artificial
  Reverse primer for amplifying human EGF
ctagatccggtggatcccctcagtggtggtggtggtggtgc

SEQ ID NO: 51 - DNA, 31 bp, Artificial
  Forward primer for amplifying mutant human KRAS having the G13D mutation
tagttggagctggtgacgtaggcaagagtgc

SEQ ID NO: 52 - DNA, 31 bp, Artificial
  Reverse primer for amplifying mutant human KRAS having the G13D mutation
gcactcttgcctacgtcaccagctccaacta

SEQ ID NO: 53 - DNA, 42 bp, Artificial
  Forward primer for amplifying mutant human KRAS having the Q61R mutation
gatattctcgacacagcaggtagagaggagtacagtgcaatg

SEQ ID NO: 54 - DNA, 42 bp, Artificial
  Reverse primer for amplifying mutant human KRAS having the Q61R mutation
cattgcactgtactcctctctacctgctgtgtcgagaatatc

SEQ ID NO: 55 - DNA, 38 bp, Artificial
  Forward primer for applying rtxA1 gene of Vibrio vulnificus
gagctagcatgggtgataaaaccaaggtcgtggattta

SEQ ID NO: 56 - DNA, 39 bp, Artificial
  Reverse primer for amplifying rtxA1 gene of Vibrio vulnificus
gccgtcgaccaaactgcccttgaacgtgatcttcggttt

(19) United States
(12) Patent Application Publication
(10) Pub. No.: US 2016/0030531 A1
(43) Pub. Date: Feb. 4, 2016

(54) BACTERIAL TOXINS AND USES THEREOF AS RAS SPECIFIC PROTEASES

(71) Applicant: Northwestern University, Evanston, IL (US)

(72) Inventors: Karla J.F. Satchell, Evanston, IL (US); Irena Antic, Chicago, IL (US); Marco Biancucci, Chicago, IL (US)

(73) Assignee: NORTHWESTERN UNIVERSITY, Evanston, IL (US)

(21) Appl. No.: 14/816,724

(22) Filed: Aug. 3, 2015

Related U.S. Application Data
(60) Provisional application No. 62/032,330, filed on Aug. 1, 2014.
(60) Provisional application No. 62/172,432, filed on Jun. 8, 2015.

(51) Int. Cl. A61K 38/48

(52) U.S. Cl. CPC A61K 38/48; C12Y 304/00

(57) ABSTRACT

Disclosed are bacterial toxins and uses thereof as specific proteases for Ras sarcoma oncoproteins (Ras proteins). The bacterial toxins may be modified for use as pharmaceutical agents for treating Ras-dependent diseases and disorders including cell proliferation diseases and disorders such as cancer.

### CROSS-REFERENCE TO RELATED PATENT APPLICATIONS

The present application claims the benefit of priority under 35 U.S.C. §119(e) to U.S. Provisional Application No. 62/172,432, filed on Jun. 8, 2015, and to U.S. Provisional Application No. 62/032,330, filed on Aug. 1, 2014, the contents of which are incorporated herein by reference in their entireties.

### STATEMENT REGARDING FEDERALLY SPONSORED RESEARCH OR DEVELOPMENT

This invention was made with government support under grant numbers RO1 AI092825 and RO1 AI098369 awarded by the National Institutes of Health. The government has certain rights in the invention.

### BACKGROUND

The field of the invention relates to bacterial toxins. In particular, the field of the invention relates to bacterial toxins that are specific proteases for Ras sarcoma oncoproteins (Ras) and uses therefor for treating Ras-dependent diseases and disorders.

Rat sarcoma (Ras) oncoproteins (e.g., KRas, HRas, and NRas) regulate cell growth, differentiation, and survival by mediating specific signal transduction within cells. Mutational activation of Ras genes is associated with 33% of human cancers, making it one of the most frequent oncogenic mutations. Cancer research has focused on developing several strategies to block mutant Ras and to inhibit the over-activation of the downstream signaling. However, three decades after the discovery of Ras, no drugs or therapeutics that target Ras proteins directly or act on Ras-driven human cancers have been developed successfully.

Here, we discovered a novel protease that cleaves Ras. This protein, known as domain of unknown function in the fifth position (DUF5), is an effector domain of the Multifunctional-Autoprocessing Repeats-in-Toxins (MARTX) family of bacterial toxins. The domain is present in the toxin secreted by some strains of the bacterial pathogen Vibrio vulnificus. This domain is also found in the MARTX toxin of other bacterial species and as a toxic domain unlinked to a MARTX toxin in other bacteria, revealing that cleavage of Ras is a conserved toxic function among various bacterial species.

When DUF5 from V. vulnificus (DUF5_{Vv}) is released into the cytosol of eukaryotic cells by natural toxin delivery from the bacterium, by transient expression following DNA transfection, or by the anthrax lethal factor N-terminal domain-protective antigen (LF_{N}-PA) delivery system, it is able to block the Ras pathway, resulting in loss of cell proliferation. Here, we demonstrate, both in vitro and in vivo, that this block occurs because DUF5_{Vv} is an endopeptidase that cleaves Ras within Switch I, an essential loop for exchange of guanosine nucleotide diphosphate (GDP) with guanosine nucleotide triphosphate (GTP) to activate Ras and for the interaction with several Ras-binding partners. The binding of guanosine nucleosides and binding partners then regulate the Ras downstream pathways that control cell growth, motility, differentiation and response to cell stress.

Because in many cancers Ras is constitutively activated by specific mutations, developing treatments against tumors harboring Ras mutations remains one of the most challenging goals in modern medicine. The use of protein toxin-based therapeutic approaches is a consolidated and alternative way of treating cancer disease compared to conventional radiation or chemical therapy. Because DUF5_{Vv} specifically cleaves Ras, including Ras mutant isoforms found in cancer cells, resulting in loss of proliferation, we have found that DUF5_{Vv} and proteins similar to DUF5_{Vv} can be used as the toxic component to create new conjugated toxin-based therapies for cancer treatment. In addition, DUF5_{Vv} can be used as a cell biological reagent to rapidly eliminate Ras from cells for research or industrial purposes.

### SUMMARY

Disclosed are bacterial toxins and uses thereof as specific proteases for Ras sarcoma oncoproteins (Ras proteins). The bacterial toxins may be modified for use as therapeutic polypeptides pharmaceutical agents for treating Ras-dependent diseases and disorders including cell proliferation diseases and disorders such as cancer.

The disclosed bacterial toxins include DUF5 proteases and active subdomains thereof that exhibit protease activity for Ras proteins, and preferably which exhibit specific protease activity for Ras proteins. Active subdomains of DUF5 proteases that exhibit protease activity for Ras proteins may include the C2A subdomain.

The disclosed bacterial toxins may be utilized in methods for treating a cell proliferative disease or disorder in a patient. Contemplated treatment methods may include administering a therapeutic polypeptide comprising a DUF5 protease or an active portion thereof comprising the C2A subdomain to the patient. Typically, the cell proliferative disease or disorder is associated with an activating mutation in a Ras protein and is a Ras-dependent cell proliferative disease or disorder such as a Ras-dependent cancer.

The disclosed bacterial toxins include the DUF5 protease, a homolog thereof, or an active portion thereof comprising the C2A subdomain from a number of microorganisms. These include, but are not limited to Vibrio vulnificus, Vibrio ordalii, Vibrio cholerae, Vibrio splendidus, Moritella dasanensis, Aeromonas salmonicida, Aeromonas hydrophila, Photorhabdus temperata, Xenorhabdus nematophila, Photorhabdus luminescens, Photorhabdus asymbiotica, Yersinia kristensenii, and Pasteurella multocida.

The disclosed bacterial toxins may be formulated as therapeutic polypeptides for delivery to the cytosol of proliferating cells. In some embodiments of the therapeutic polypeptides, the DUF5 protease, a homolog thereof, or a portion thereof comprising the C2A subdomain may be fused or complexed with a carrier that facilitates transport of the DUF5 protease, the homolog thereof, or the C2A subdomain thereof into the cytosol of proliferating cells.

Pharmaceutical compositions and kits comprising the disclosed bacterial toxins for treating cell proliferative diseases or disorders also are contemplated herein. The compositions may include a therapeutic polypeptide comprising a DUF5 protease, a homolog thereof, or a portion thereof comprising the C2A subdomain, and a carrier that facilitates transport of the DUF5 protease, the homolog thereof, or the portion thereof comprising the C2A subdomain into the cytosol of proliferating cells. In the therapeutic polypeptides of the compositions and kits, the DUF5 protease, the homolog thereof, or the portion thereof comprising the C2A subdomain may be fused or conjugated to the carrier or complexed with the carrier. Specifically contemplated are fusion proteins comprising the amino acid sequence of the disclosed bacterial toxins fused to the amino acid sequence of a carrier polypeptide that facilitates transport of the bacterial toxins into proliferating cells.

### BRIEF DESCRIPTION OF THE FIGURES

FIG. 1. Vibrio vulnificus CMCP6 MARTX toxin CMCP6. (A) Linear schematic shows the overall domain structure of the toxin with the pore forming conserved regions in grey and the autoprocessing cysteine protease (CPD) in blue. The cytotoxic and cytopathic “effector domains” are DUF1, RID, ABH, MCF, and DUF5 are described in text. (B) The current model for toxin assembly on the eukaryotic cell membrane to form a pore for translocation of the central domains. After being translocated, the CPD binds inositol hexakisphosphate (InsP6) to initiate autoprocessing between effector domains for release to the cytosol. The five domains are then free to access targets in the cell.

FIG. 2. Left: DUF5_{Vv} crystals. Right: Domain structure of DUF5_{Vv} based on crystal structure. C1/MLD (blue): membrane localization domain; C2A (green): Smallest active domain; C2B (brown): Putative stabilization or specificity domain.

FIG. 3. Cells intoxicated with DUF5_{Vv} or C1/C2A show loss of all cellular Ras (A) Western blot to detect total ERK1/2 (upper panels) or phosphor-ERK1/2 (lower panels). Cellular actin in whole cell lysate was used as the loading control. Prior to collection, HeLa cell were incubated for 24 hr with protective antigen (PA), the N-terminus of Lethal factor (LFn), DUF5_{Vv} fused to anthrax toxin lethal factor (LF_{N}DUF5), or mixture of proteins as shown. (B) GLISA activation assay (Cytoskeleton Inc) to quantify total active Ras in the GTP bound form (% active Ras) from Hela cell lysates intoxicated as in panel A. (C) Detection of total Ras in cell lysates by western blot using Ras10 monoclonal antibody (upper panels). Detection with anti-actin antibody was used as the loading control.

FIG. 4. HeLa cells intoxicated with DUF5_{Vv} or C1/C2A show loss of all cellular Ras. Detection of total Ras in cell lysates by western blot using Ras10 monoclonal antibody (upper panels). Detection with anti-actin antibody was used as the loading control.

FIG. 5. Cells intoxicated with DUF5 show rapid loss of detectable Ras. Western blot detection of total Ras using Ras10 antibody (Upper panel). Prior to collection, HeLa cell were incubated for time shown with DUF5 fused to anthrax toxin lethal factor (LF_{N}DUF5) in the absence (first lane) or presence of PA.

FIG. 6. Intoxication of cells with DUF5_{Vv} results in truncation of H-Ras. (A) HeLa cells were transfected to overexpress HA-tagged HRas and then intoxicated with LF_{N}DUF5_{Vv}/PA for 24 hr. HA-HRas was immunoprecipitated with anti-HA peptide agarose beads and bound protein was eluted from the bead, separated by SDS-PAGE and visualized using Coomassie Brilliant blue. (B) The 18 kDa band was excised and subjected to peptide mapping by mass spectrometry. Peptides matched to H-Ras region shown in grey. (C) Western blot analysis on IP elution fractions using both anti-HA antibody to detect full length expression product and an antibody specific to C-terminus of H-Ras to verify this protein is H-Ras from which the N-terminus comprised of the HA and Ras10 epitopes is absent.

FIG. 7. Intoxication of cells with DUF5_{Vv} results in truncation of all Ras isoforms. HeLa cells were transfected to overexpress HA-tagged Ras isoforms as indicated and then intoxicated with LF_{N}DUF5_{Vv}/PA for 24 hr. Western blot analysis on HeLa whole cell lysates transfected with (A) HA-KRas, (B) HA-NRas and (C) HA-HRas. Cells were either untreated (−) or intoxicated with LF_{N}DUF5_{Vv} in combination with PA (+).

FIG. 8. DUF5_{Vv} directly cleaves Ras isoforms in vitro. Reactions of rDUF5_{Vv} recombinant Ras isoforms as indicated (1:1 molar ratio) was performed in 50 mM TRIS, 10 mM MgCL₂, 500 mM NaCl pH 7.5 at 37° C. Nucleotides were added as shown. After 10 minutes of incubation, each sample reaction was stopped by addition of 6×SDS-PAGE Loading buffer and boiling for 5 min. Samples were separated on 15% SDS-PAGE gel and bands were visualized with Coomassie brilliant blue.

FIG. 9. DUF5_{Vv} directly cleaves K-Ras in vitro. A reaction of rDUF5_{Vv} with KRas performed in FIG. 8 in the presence of guanosine nucleotides as indicated show no dependence on nucleotide for proper conformation of rKRas in this reaction.

FIG. 10. DUF5_{Vv} cleaves Ras isoforms between Y32 and D33. Bands in FIG. 8 were excised and N-terminal sequence determined by Edman degradation. All isoforms cleaved the same site shown by arrows.

FIG. 11. rKRas is cleaved by DUF5 from A. hydrophila (DUF5_{Ah}) and by P. asymbiotica hypothetical protein PAT3833 (DUF5_{Pa}). A reaction of rDUF5_{Vv} with KRas performed in FIG. 8 show that other proteins with homology to DUF5_{Vv} can also cleave rKRas in vitro.

FIG. 12. Other small GTPase proteins are not cleaved by DUF5_{Vv} A reaction performed as in FIG. 8 with rDUF5_{Vv} with small GTPases proteins purified as fusions to glutathione-S-transfer as indicated. No other small GTPases were cleaved by DUF5_{Vv}.

FIG. 13. LF_{N}DUF5_{Vv} is toxic to colorectal and breast cancer cell lines. Cells were treated with LFNDUF5_{Vv} in the presence of PA and cytotoxicity was observed.

FIG. 14. rKRas G12V is cleaved by DUF5_{Vv} A reaction of rDUF5_{Vv} with rKRas bearing the common G12V mutation was performed as in FIG. 8. These data show that DUF5_{Vv} can also mutant forms of rKRas that are common in cancer.

FIG. 15. DUF5 from V. vulnificus MARTX toxin is cytotoxic to HeLa cells. (A) Scale drawing of V. vulnificus MARTX and P. multocida PMT protein toxins with enlarged region showing C1, C2A, and C2B domains that are shared between the two toxins (B-F) Epifluorescent and DIC images (200×) of HeLa epithelial cells transfected with pEGFP-N3 plasmid clones expressing EGFP (B), DUF5_{Vv}-EGFP (C-E), or C1C2Pm-EGFP (F). Panels (D) and (E) enlarged 200% to show detail of localization of DUF5_{Vv}-EGFP and cell blebbing, respectively. Arrows in panel E indicate EGFP-positive cells in DIC only image. Percent of rounded cells in each cell type is quantified from three independent experiments (G) and expression of protein in transfected cells is shown by western blot detection using an anti-GFP antibody (H).

FIG. 16A, FIG. 16B, FIG. 16C, FIG. 16D, FIG. 16E and FIG. 16F. The C1 MLD of DUF5_{Vv} is necessary only for efficient cell rounding. (FIG. 16A) Structural model of DUF5_{Vv} generated in HHPRED and Modeller based on published structure of PMT. C1, C2A, and C2B subdomains are indicated. (FIG. 16B-FIG. 16D) Epifluorescent and DIC images (200×) of HeLa epithelial cells transfected with pEGFP-N3 plasmid clones expressing EGFP (FIG. 16B), DUF5_{Vv}-EGFP (FIG. 16C), or C2-GFP (FIG. 16E) and C2B-EGFP (FIG. 16F).

FIG. 17A, FIG. 17B, FIG. 17C, FIG. 17D, FIG. 17E, FIG. 17F, FIG. 17G, FIG. 17H, FIG. 17I, FIG. 17J and FIG. 17K. C2A is the cytotoxic subdomain of DUF5_{Vv}. (FIG. 17A). Schematic of proteins expressed in the panel. (FIG. 17B-FIG. 17H) Epifluorescent and DIC images (200×) of HeLa epithelial cells transfected with pEGFP-N3 plasmid clones expressing EGFP (B), DUF5_{Vv}-EGFP (FIG. 17C), C2-EGFP (FIG. 17D), C2A-EGFP (FIG. 17E) and C2B-EGFP (FIG. 17F). Average of percent rounded cells in each cell type is quantified from three independent experiments (FIG. 17G) and expression of protein in transfected cells is shown by western blot detection using and anti-GFP antibody (FIG. 17H). Note that C2A-EGFP could not be detected due to consistent poor sample recovery from plates due to toxicity of this domain. (FIG. 17I, FIG. 17J, FIG. 17K) DIC images of HeLa cells intoxicated with 7 nM PA in combination with 3 nM purified unmodified LFN (I), LFN fused to DUF5_{Vv} (FIG. 17J) and LFN fused to only the C1-C2A subdomains of DUF5_{Vv} (FIG. 17K).

FIG. 18A, FIG. 18B, FIG. 18C, FIG. 18D, FIG. 18E, FIG. 18F and FIG. 18G. DUF5 from A. hydrophila is cytotoxic and causes cell rounding. LFN-DUF5_{Ah} caused cell rounding when delivered to HeLa cells (FIG. 18A-FIG. 18B). Protein purity was assessed with SDS-PAGE (FIG. 18C). Rounding efficiency was comparable to DUF5_{Vv}, at all concentrations tested (FIG. 18D, FIG. 18E). Finally, release of LDH from intoxicated cells was measured (F, G), showing that there is no appreciable lysis when cells are intoxicated with either DUF5 protein.

FIG. 19. Amino acid alignment generated in MacVector 12.6.0 of only the C2A and C2B subdomains. Grey shading indicates 100% identical residues. Triangles indicate that sections of sequence were removed during alignment calculations. Asterisk indicated residues changed to alanine via site directed mutagenesis and boxes indicate two aa identified as important for growth inhibition in yeast. Larger asterisks indicate residues G3948 and V3906 which were mutated to stop codons, while the last large asterisk indicates S3986, which was not targeted in the initial mutagenesis but was later found by structural modeling to potentially interact with R3841.

FIG. 20A, FIG. 20B and FIG. 20C. DUF5_{Vv} and the C2 domain cause growth inhibition when expressed in yeast (FIG. 20A, FIG. 20B, and FIG. 20C. S. cerevisiae strain InvSc1 was transformed with pYC2 NT/A plasmid expressing proteins indicated at left or with empty vector (EV), actin crosslinking domain from V. cholerae MARTX (ACD), or the C2 domain with stop codons introduced at V3906 or G3048. Panels show 5 μl of 10-fold serial dilutions spotted to SC agar without uracil supplemented with either glucose (non-inducing) or galactose and raffinose (inducing). Panels at right show 12 h growth curves of cultures in SC broth with galactose.

FIG. 21A, FIG. 21B, FIG. 21C, FIG. 21D, FIG. 21E and FIG. 21F. D3721 and R3841 are important residues for growth inhibition of yeast. Growth inhibition in yeast for C2-D3721A and C2-D3721E (FIG. 21A) and DUF5_{Vv}-D3721A, DUF5_{Vv}-R3841A, DUF5 with both residues mutated to alanine (DARA) and DUF5 with swapped residues (DRRD) in panel B. See FIG. 17 for details. (FIG. 21C, FIG. 21D) Structural model of DUF5_{Vv} showing polar contacts of D3721 and R3841 and potential cross association of R3841 with 53986 in C2B. Color-coding is the same as in FIG. 15A. Panel E shows the purified 6×His-tagged DUF5 and DUF5 D3721A proteins that were used in FTS experiments to determine melting temperature in panel F.

FIG. 22A, FIG. 22B, FIG. 22C, FIG. 22D, FIG. 22E, FIG. 22F and FIG. 22G. D3721 and R3841 are important residues for intoxication of HeLa cells. (FIG. 22A-FIG. 22E) DIC images of HeLa cells intoxicated for 24 h (upper) or 48 h (lower) with 7 nM PA in combination with 3 nM purified unmodified LFN (FIG. 22A), LFN fused to DUF5_{Vv} (FIG. 22B) and LFN fused to only the C1-C2A subdomains of DUF5_{Vv} (FIG. 22C), or LFN fused to only the C1-C2A subdomains of DUF5_{Vv} with D3721 (FIG. 22D) or R3841 (FIG. 22E) point mutations. Protein purity was assessed by SDS-PAGE in panel F. Three independent experiments were performed and cells were manually counted (FIG. 22G).

FIG. 23. MARTX toxin undergoes autoprocessing upon entry into the host cell. Autoprocessing by the inositol hexakisphosphate bound cysteine protease domain releases other effector domains and allows them to perform their functions. DUF5 has been shown to be a stable protein when all the subdomains are present, and is able to efficiently round cells when the C2 domain is intact. When C2B subdomain in removed from the protein, leaving only C1-C2A, cell rounding is less efficient, presumably due to protein turnover. Therefore, C2B is hypothesized to be involved in stabilizing the interaction between DUF5 and the cellular target. C2A and C2B are required for a stable interaction with the target protein, but C2A alone is sufficient for cytotoxic activity.

FIG. 24A, FIG. 24B, FIG. 24C, FIG. 24D and FIG. 24E. DUF5_{Vv}-dependent disruption of Ras-ERK-dependent cell proliferation. (FIG. 24A) Major categories of yeast mutants enabling growth in the presence of DUF5_{Vv}-C2. (FIG. 24B, FIG. 24D) Representative immunoblots (n=3) of lysates prepared from cells treated for 24 h (b) or time indicated (d) with LFNDUF5_{Vv} in the absence 0 or the presence (+) of PA. Trimmed ERK1/2 blots are shown unedited in FIG. 29. (FIG. 24C, FIG. 24E) Clonogenic colony-formation assay (n=2) of cells treated for 24 (FIG. 24C) or 1 h (FIG. 24E). Error bars represent the range of the data.

FIG. 25A, FIG. 25B, FIG. 25C, FIG. 25D, FIG. 25E and FIG. 25F. DUF5_{Vv} is a Ras site-specific endopeptidase. (FIG. 25A) Coomassie-stained 18% SDS-polyacrylamide gel of anti-HA immunoprecipitated proteins from cells expressing HA-HRas treated for 24 h as indicated. Lower band (HRas*) was excised for peptide sequencing with HRas peptide coverage highlighted in yellow. (FIG. 25B) Same fractions probed by immunoblotting to detect the N terminus (anti-HA) and C terminus (isotype-specific antibody). (FIG. 25C) Lysates from cells expressing HA-tagged KRas, NRas or HRas probed by immunoblotting as indicated. (FIG. 25D) In-vitro cleavage of 10 mM rKRas to KRas* with 10 mM rDUF5_{Vv} (inset) or concentration indicated. Error bars indicate mean±s.d. (n=3). (FIG. 25E) In-vitro cleavage of 10 mM rKRas, rHRas and rNRas with 10 mM rDUF5_{Vv}. Identical results of Edman degradation were obtained for all three proteins. (FIG. 25F). Black arrow indicates the cleavage site in the Switch I region (red) of HRas69.

FIG. 26A, FIG. 26B, FIG. 26C, FIG. 26D, FIG. 26E and FIG. 26F. DUF5 homologues and other GTPase substrates. (FIG. 26A) Schematic diagram of DUF5 (orange) within the mosaic architecture of effector domains in MARTX toxins from V. vulnificus (_{Vv}), A. hydrophila (Ah), Vibrio splendidus (Vs), Xenorhabdus nematophila (_{Xn}) and Yersinia kristensii (_{Yk}) or as stand-alone proteins in Photorhabdus luminescens (_{Pl}) and P. asymbiotica (_{Pa}) as previously described^{17,20}. (FIG. 26B) In-vitro cleavage of 10 mM KRas with 10 mM rDUF5 from various species. (FIG. 26C) LFNDUF5 tested for in-vivo loss of all Ras isoforms after 24 h under the same conditions as in b. (FIG. 26D) Amino acid identity in Switch I regions of representative GTPases (left) from five major Ras families (right). (FIG. 26E) Bar graph of percent GFP-fusion protein cleaved after delivery of LFNDUF5_{Vv}+PA, quantified from immunoblots (FIG. 34). Error bars indicate mean±s.d. (n=3). (FIG. 26F) Representative in-vitro cleavage (n=3) of GST-fusion proteins to release GST*. Negative cleavage reactions for nine other substrates are shown in FIG. 35.

FIG. 27A, FIG. 27B, FIG. 27C and FIG. 27D. DUF5_{Vv} during bacterial infection and as a potential treatment of malignancies. (FIG. 27A) MARTX toxin effector domain configuration in V. vulnificus isolates CMCP6 (DUF5_{Vv}+) and M06-24/O (DUF5_{Vv}−). (FIG. 27B) Representative immunoblots (n=2) of lysates from cells incubated with V. vulnificus as indicated and probed for Ras cleavage and ERK1/2 dephosphorylation. (FIG. 27C) Phase-contrast images and immunoblot detection of Ras from HCT116 and MDAMB-231 cells treated as indicated for 24 h. (FIG. 27D) In-vitro processing of 10 mM rKRas with mutations as indicated.

FIG. 28. Schematic summary of yeast deletion screen. (a) Diagram of pYC-C2 plasmid expressing DUF5Vv-C2 under control of the GAL1 galactoseinducible promoter. (b) Plating efficiency of S. cerevisiae InvSc2 expressing DUF5Vv-C2 (C2Vv) compared to yeast transformed with empty vector (EV) and the more toxic full-length DUF5Vv and actin crosslinking domain from V. cholerae (ACDVc), which eliminates the actin cytoskeleton (Geissler B, et al. Mol Microbiol 73, 858-868 (2009)). (c) Schematic showing the arrayed library of non-essential deletion strains transformed with pYC-C2, followed by selection on glucose to repress expression of DUF5Vv-C2. The resulting yeast colonies were patched onto galactose and raffinose to induce expression. (d) Plate 24 of the library, showing the initial screen yeast transformed with empty vector (C) and strains selected for secondary screening by quantitative plating (circled).

FIG. 29. DUF5Vv inhibits ERK1/2 phosphorylation, but not p38. (a,b) Representative immunoblots (n=2) of lysates from cells treated as indicated for 24 h. Red boxes highlight differences in phospho-p38 (pp38) and phospho-ERK1/2 (pERK1/2) levels. Note that Panel b is the same figure from which lanes were removed to align with other western blots in FIG. 24.

FIG. 30. HeLa cells treated with DUF5Vv lack active (GTP-bound) Ras. Bar graph of relative detection of active GTP-bound Ras (all isoforms) by G-LISA. Failure to detect active Ras was ultimately explained by the complete absence of Ras detectable by the monoclonal RAS10 antibody provided with the assay kit.

FIG. 31. Ras inactivation by DUF5_{Vv} occurs rapidly Immunoblot of lysates from cells treated for time indicated. Control samples (first four lanes) were collected 30 minutes after intoxication.

FIG. 32. DUF5Vv specificity against GFP-tagged small GTPases. HEK 293T cells transfected to express small GTPases with N-terminal EGFP-fusion as indicated were either untreated (−) or intoxicated with LFNDUF5Vv in combination with PA (+) for 24 h, at which time cell lysates were probed with anti-EGFP antibody. For triplicate blots, GFP* and GFP-x bands were quantified by Image J 1.64 and percent cleavage determined as GFP*/(GFP*+GFP-x). For FIG. 26, samples were normalized to untreated cells to account for closely sized non-specific bands or natural breakdown. Raw pixel data is shown in table.

FIG. 33. DUF5Vv specificity against GST-tagged small GTPases. In vitro processing of 10 μM purified small GTPases with N-terminal fusion of GST (GST-x) to two fragments (GST* and GTPase*) by 10 μM rDUF5Vv for 10 min. This extended figure shows representative data (n=3). Only the positive samples, HRas and Rap1A, are duplicated in FIG. 26F.

FIG. 34. HeLa cell rounding and lysis due to V. vulnificus. V. vulnificus MARTX toxins have distinct compositions dependent upon the strain isolate, as shown in FIG. 27. Representative (n=3) phase images of cell rounding (a) and LDH release (b) induced after 60 min co-incubation of bacteria as indicated with HeLa cells, at which point cells were collected for detection of Ras and pERK in FIG. 27. (c) Cell lysis over time after addition of bacteria. Note that after 3 h, even bacteria without rtxA1 induce cell lysis due to the vvhAencoded cytolysin/hemolysin (Fan et al. Infect Immun 69, 5943-5948 (2001)). Error bars represent mean±standard deviation.

FIG. 35. Malignant cells are affected by DUF5Ah from A. hydrophila. Phase images and immunoblot detection of Ras from HCT116 and MDA-MB-231 treated as indicated for 24 h.

### DETAILED DESCRIPTION

The present invention is described herein using several definitions, as set forth below and throughout the application.

As used in this specification and the claims, the singular forms “a,” “an,” and “the” include plural forms unless the context clearly dictates otherwise. For example, the term “a protease” should be interpreted to mean “one or more proteases” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

As used herein, the term “patient” may be used interchangeably with the term “subject” or “individual” and may include an “animal” and in particular a “mammal” Mammalian subjects may include humans and other primates, domestic animals, farm animals, and companion animals such as dogs, cats, guinea pigs, rabbits, rats, mice, horses, cattle, cows, and the like.

As used herein, the term “biological sample” should be interpreted to include bodily fluids (e.g., blood, serum, plasma, saliva, urine samples) and body tissue samples. Suitable tissue samples may include tissue samples from cancerous tissues and tumors.

The disclosed methods, compositions, and kits may be utilized to treat a patient in need thereof. A “patient in need thereof” is intended to include a patient having or at risk for developing diseases and disorders such as cell proliferative diseases and disorders which may include cancer and hyperproliferative disorders. A patient in need thereof may include a patient having or at risk for developing any of adenocarcinoma, leukemia, lymphoma, melanoma, myeloma, sarcoma, and teratocarcinoma, (including cancers of the adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus).

The bacterial toxins disclosed herein may include a DUF5 protease, a homolog thereof, or a subdomain thereof such as the C2A subdomain of the DUF5 protease. The disclosed bacterial toxins may include polypeptides derived from a number of microorganisms, including, but not limited to Vibrio vulnificus, Vibrio harveyi, Vibrio ordalii, Vibrio cholerae, Vibrio splendidus, Moritella dasanensis, Aeromonas salmonicida, Aeromonas hydrophila, Photorhabdus temperata, Xenorhabdus nematophila, Photorhabdus luminescens, Photorhabdus asymbiotica, Yersinia kristensenii, and Pasteurella multocida.

As utilized herein, a protein, polypeptide, and peptide refer to a molecule comprising a chain of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

The amino acid sequence of the DUF5 protease of Vibrio vulnificus is provided as SEQ ID NO:1, and the amino acid sequence of the C2A subdomain is provided as SEQ ID NO:2. The amino acid sequence of the DUF5 protease of Vibrio harveyi is provided as SEQ ID NO:3, and the amino acid sequence of the C2A subdomain is provided as SEQ ID NO:4. The amino acid sequence of the DUF5 protease of Vibrio ordali is provided as SEQ ID NO:5, and the amino acid sequence of the C2A subdomain is provided as SEQ ID NO:6. The amino acid sequence of the DUF5 protease of Vibrio cholera is provided as SEQ ID NO:7, and the amino acid sequence of the C2A subdomain is provided as SEQ ID NO:8. The amino acid sequence of the DUF5 protease of Vibrio splendidus is provided as SEQ ID NO:9, and the amino acid sequence of the C2A subdomain is provided as SEQ ID NO:10. The amino acid sequence of the DUF5 protease of Moritella desanensis is provided as SEQ ID NO:11, and the amino acid sequence of the C2A subdomain is provided as SEQ ID NO:12. The amino acid sequence of the DUF5 protease of Aeromonas salmonicida is provided as SEQ ID NO:13, and the amino acid sequence of the C2A subdomain is provided as SEQ ID NO:14. The amino acid sequence of the DUF5 protease of Aeromonas hydrophila is provided as SEQ ID NO:15, and the amino acid sequence of the C2A subdomain is provided as SEQ ID NO:16. The amino acid sequence of the DUF5 protease of Photorabdus temperata is provided as SEQ ID NO:17, and the amino acid sequence of the C2A subdomain is provided as SEQ ID NO:18. The amino acid sequence of the DUF5 protease of Xenerhabdus nematophila is provided as SEQ ID NO:19, and the amino acid sequence of the C2A subdomain is provided as SEQ ID NO:20. The amino acid sequence of the DUF5 protease of Photorabdus luminescences is provided as SEQ ID NO:21, and the amino acid sequence of the C2A subdomain is provided as SEQ ID NO:22. The amino acid sequence of the DUF5 protease of Photorabdus asymbiotica is provided as SEQ ID NO:23, and the amino acid sequence of the C2A subdomain is provided as SEQ ID NO:24. The amino acid sequence of the DUF5 protease of Yersinia kristensenii is provided as SEQ ID NO:25, and the amino acid sequence of the C2A subdomain is provided as SEQ ID NO:26. The amino acid sequence of the DUF5 protease homolog of Pasteurella multocida is provided as SEQ ID NO:27, and the amino acid sequence of the C2A subdomain is provided as SEQ ID NO:28.

The terms “amino acid” and “amino acid sequence” refer to an oligopeptide, peptide, polypeptide, or protein sequence (which terms may be used interchangeably), or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

The amino acid sequences contemplated herein may include conservative amino acid substitutions relative to a reference amino acid sequence. For example, a variant, mutant, or derivative polypeptide may include conservative amino acid substitutions relative to a reference polypeptide. “Conservative amino acid substitutions” are those substitutions that are predicted to interfere least with the properties of the reference polypeptide. In other words, conservative amino acid substitutions substantially conserve the structure and the function of the reference protein. The following Table provides a list of exemplary conservative amino acid substitutions.

| Original | Conservative |
|---|---|
| Residue | Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid sequence that results in the absence of one or more amino acid residues. A deletion removes at least 1, 2, 3, 4, 5, 10, 20, 50, 100, or 200 amino acids residues. A deletion may include an internal deletion or a terminal deletion (e.g., an N-terminal truncation or a C-terminal truncation of a reference polypeptide). A “variant,” “mutant,” or “derivative” of a reference polypeptide sequence may include a deletion relative to the reference polypeptide sequence.

The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues. A “variant,” “mutant,” or “derivative” of a reference polypeptide sequence may include an insertion or addition relative to the reference polypeptide sequence.

A “fusion polypeptide” refers to a polypeptide, such as the bacterial toxins contemplated herein, comprising at the N-terminus, the C-terminus, or at both termini of its amino acid sequence a heterologous amino acid sequence, for example, an amino acid sequence that facilitates transport of the polypeptide into the cytosol of proliferating cells. A “variant,” “mutant,” or “derivative” of a reference polypeptide sequence may include an fusion polypeptide comprising the reference polypeptide fused to a heterologous polypeptide.

A “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full length polypeptide. A “variant,” “mutant,” or “derivative” of a reference polypeptide sequence may include a fragment of the reference polypeptide sequence.

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “variant,” “mutant,” or “derivative” of a particular polypeptide sequence may be defined as a polypeptide sequence having at least 20% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

A “variant,” “mutant” or a “derivative” may have substantially the same functional activity as a reference polypeptide. For example, a variant, mutant, or derivative of a DUF5 protease or the C2A subdomain thereof may function as a protease of a Ras protein, for example, and specifically cleave the Ras protein between a tyrosine at amino acid position 32 and an aspartic acid at amino acid position 33 of the amino acid sequence of the Ras protein.

A protein, polypeptide, or peptide as contemplated herein may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

Also contemplated herein are peptidomimetics of the disclosed proteins, polypeptides, and peptides. As disclosed herein, a peptidomimetic is an equivalent of a protein, polypeptide, or peptide characterized as retaining the polarity, three dimensional size and functionality (bioactivity) of the protein, polypeptide, or peptide equivalent but where the protein, polypeptide, or peptide bonds have been replaced (e.g., by more stable linkages which are more resistant to enzymatic degradation by hydrolytic enzymes). Generally, the bond which replaces the amide bond conserves many of the properties of the amide bond (e.g., conformation, steric bulk, electrostatic character, and possibility for hydrogen bonding). A general discussion of prior art techniques for the design and synthesis of peptidomimetics is provided in “Drug Design and Development”, Chapter 14, Krogsgaard, Larsen, Liljefors and Madsen (Eds) 1996, Horwood Acad. Pub, the contents of which are incorporated herein by reference in their entirety. Suitable amide bond substitutes include the following groups: N-alkylation (Schmidt, R. et. al., Int. J. Peptide Protein Res., 1995, 46, 47), retro-inverse amide (Chorev, M and Goodman, M., Acc. Chem. Res, 1993, 26, 266), thioamide (Sherman D. B. and Spatola, A. F. J. Am. Chem. Soc., 1990, 112, 433), thioester, phosphonate, ketomethylene (Hoffman, R. V. and Kim, H. O. J. Org. Chem., 1995, 60, 5107), hydroxymethylene, fluorovinyl (Allmendinger, T. et al., Tetrahydron Lett., 1990, 31, 7297), vinyl, methyleneamino (Sasaki, Y and Abe, J. Chem. Pharm. Bull. 1997 45, 13), methylenethio (Spatola, A. F., Methods Neurosci, 1993, 13, 19), alkane (Lavielle, S. et. al., Int. J. Peptide Protein Res., 1993, 42, 270) and sulfonamido (Luisi, G. et al. Tetrahedron Lett. 1993, 34, 2391), which all are incorporated herein by reference in their entireties. Contemplated herein are peptidomimetic equivalents of the disclosed therapeutic polypeptides comprising the amino acid sequence of a DUF5 protease C2A subdomain.

Also disclosed herein are polynucleotides, for example polynucleotide sequences that encode the polypeptides and proteins disclosed herein (e.g., DNA that encodes a polypeptide having the amino acid sequence of any of SEQ ID NOs:1-28 or DNA that encodes a polypeptide variant having an amino acid sequence with at least about 20%, 30%, 40%, 50%, 60%, 70%, 80%, 90%, 95%, 96%, 97%, 98%, or 99% sequence identity to any of SEQ ID NOs: 1-28.

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

“Transformation” or “transfected” describes a process by which exogenous nucleic acid (e.g., DNA or RNA) is introduced into a recipient cell. Transformation or transfection may occur under natural or artificial conditions according to various methods well known in the art, and may rely on any known method for the insertion of foreign nucleic acid sequences into a prokaryotic or eukaryotic host cell. The method for transformation or transfection is selected based on the type of host cell being transformed and may include, but is not limited to, bacteriophage or viral infection or non-viral delivery. Methods of non-viral delivery of nucleic acids include lipofection, nucleofection, microinjection, electroporation, heat shock, particle bombardment, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term “transformed cells” or “transfected cells” includes stably transformed or transfected cells in which the inserted DNA is capable of replication either as an autonomously replicating plasmid or as part of the host chromosome, as well as transiently transformed or transfected cells which express the inserted DNA or RNA for limited periods of time.

The polynucleotide sequences contemplated herein may be present in expression vectors. For example, the vectors may comprise: (a) a polynucleotide encoding an ORF of a protein; (b) a polynucleotide that expresses an RNA that directs RNA-mediated binding, nicking, and/or cleaving of a target DNA sequence; and both (a) and (b). The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed. For example, a heterologous promoter for a LAMP may include a eukaryotic promoter or a prokaryotic promoter that is not the native, endogenous promoter for the LAMP

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide.

Any of the conventional vectors used for expression in eukaryotic cells may be used for directly introducing DNA into a subject. Expression vectors containing regulatory elements from eukaryotic viruses may be used in eukaryotic expression vectors (e.g., vectors containing SV40, CMV, or retroviral promoters or enhancers). Exemplary vectors include those that express proteins under the direction of such promoters as the SV40 early promoter, SV40 later promoter, metallothionein promoter, human cytomegalovirus promoter, murine mammary tumor virus promoter, and Rous sarcoma virus promoter. Expression vectors as contemplated herein may include eukaryotic or prokaryotic control sequences that modulate expression of a heterologous protein (e.g. the fusion protein disclosed herein). Prokaryotic expression control sequences may include constitutive or inducible promoters (e.g., T3, T7, Lac, trp, or phoA), ribosome binding sites, or transcription terminators.

The vectors contemplated herein may be introduced and propagated in a prokaryote, which may be used to amplify copies of a vector to be introduced into a eukaryotic cell or as an intermediate vector in the production of a vector to be introduced into a eukaryotic cell (e.g. amplifying a plasmid as part of a viral vector packaging system). A prokaryote may be used to amplify copies of a vector and express one or more nucleic acids, such as to provide a source of one or more proteins for delivery to a host cell or host organism. Expression of proteins in prokaryotes may be performed using Escherichia coli with vectors containing constitutive or inducible promoters directing the expression of either a protein or a fusion protein comprising a protein or a fragment thereof. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; (iii) to aid in the purification of the recombinant protein by acting as a ligand in affinity purification (e.g., a His tag); (iv) to tag the recombinant protein for identification (e.g., such as Green fluorescence protein (GFP) or an antigen (e.g., HA) that can be recognized by a labelled antibody); (v) to promote localization of the recombinant protein to a specific area of the cell (e.g., where the protein is fused (e.g., at its N-terminus or C-terminus) to a nuclear localization signal (NLS) which may include the NLS of SV40, nucleoplasmin, C-myc, M9 domain of hnRNP A1, or a synthetic NLS). The importance of neutral and acidic amino acids in NLS have been studied. (See Makkerh et al. (1996) Curr Biol 6(8):1025-1027). Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

The presently disclosed methods may include delivering one or more polynucleotides, such as or one or more vectors as described herein, one or more transcripts thereof, and/or one or proteins transcribed therefrom, to a host cell. Further contemplated are host cells produced by such methods, and organisms (such as animals, plants, or fungi) comprising or produced from such cells. The disclosed exosomes may be prepared by introducing vectors that express mRNA encoding a fusion protein and a cargo RNA as disclosed herein. Conventional viral and non-viral based gene transfer methods can be used to introduce nucleic acids in mammalian cells or target tissues. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell.

In the methods contemplated herein, a host cell may be transiently or non-transiently transfected (i.e., stably transfected) with one or more vectors described herein. In some embodiments, a cell is transfected as it naturally occurs in a subject (i.e., in situ). In some embodiments, a cell that is transfected is taken from a subject (i.e., explanted). In some embodiments, the cell is derived from cells taken from a subject, such as a cell line. Suitable cells may include stem cells (e.g., embryonic stem cells and pluripotent stem cells). A cell transfected with one or more vectors described herein may be used to establish a new cell line comprising one or more vector-derived sequences. In the methods contemplated herein, a cell may be transiently transfected with the components of a system as described herein (such as by transient transfection of one or more vectors, or transfection with RNA), and modified through the activity of a complex, in order to establish a new cell line comprising cells containing the modification but lacking any other exogenous sequence.

DUF5 Domain of Multifunctional, Autoprocessing RTX (MARTX) Toxin

Bacterial toxins can inactive host cellular processes. Ras is a cellular protein for the host response to stress that is modified in many human cancers to promote cell survival. We discovered that the DUF5 domain of the Vibrio vulnificus multifunctional, autoprocessing RTX (MARTX) toxin is an endopeptidase that specifically cleaves Ras between residues Y32 and D33. We determined the crystal structure, the minimally active portion, and the specificity for Ras among the small GTPases. Ras proteins with mutations common in cancer are also cleaved. We also have utilized a system to deliver the domain to cells independent of the holotoxin by fusion to Anthrax toxin Lethal Factor N-terminus and delivery to cells by Protective antigen. Homology sequence analysis demonstrates that at least 12 bacteria produce a protein with at least 24% percent identity and the proteins from Aeromonas hydrophila and Photorhabdus asymbiotica are shown also to cleave Ras. We propose this family of proteins could be engineered for delivery to cancer cells as potential therapeutic agents for carcinoma, targeting both tumors with normal Ras and those with modified Ras proteins. We propose this family of proteins also for use in biological research to specifically and rapidly knock down or remove Ras.

Ras-Dependent Cancers

Ras-activating mutations are frequently observed in cancer. (See Fernandez-Medarde et al., “Ras in Cancer and Developmental Diseases,” March 2011, vol. 2, no. 3: 344-358; Johannes L. Bos. “Ras Oncogenes in Human Cancer: A Review,” Cancer Research 49, 4682-4689, Sep. 1, 1989; Julian Downward “Targeting RAS signalling pathways in cancer therapy,” Nature Reviews Cancer 3, 11-22 (January 2003); Schubbert et al., “Hyperactive Ras in developmental disorders and cancer,” Nature Reviews Cancer 7, 295-308 (January 2007); and Baines et al., “Inhibition of Ras for cancer treatment: the search continues,” Future Med. Chem. 2011 October; 3(14): 1787-1808; the contents of which are incorporated herein by reference in their entireties). Ras-activating mutations are observed in 95% of pancreatic cancers, 45% of colorectal cancers, and 35% of lung adenocarcinoma. The RAS oncogenes (HRAS, NRAS and KRAS comprising activating mutations present in codon 12, 13, or 61) comprise the most frequently mutated class of oncogenes in human cancers (33%), stimulating intensive effort in developing anti-Ras inhibitors for cancer treatment. (See Prior et al., “A comprehensive survey of Ras mutations in cancer,” Cancer Research. 2012 May 15; 72(10): 2457-2467, the content of which is incorporated herein by reference in its entirety). Unfortunately, there are no drugs that target Ras directly or indirectly, and there are currently no effective therapies for Ras-dependent cancers.

Targeted Delivery or Expression of Bacterial Toxins into the Cytosol of Proliferating Cells

The bacterial toxins disclosed herein may be administered in order to treat cell proliferative diseases and disorders such as cancer. The bacterial toxins may be administered by transfecting cancer cells with a polynucleotide or a polynucleotide vector that expresses the bacterial toxins in the cancer cells. Alternatively, the bacterial toxins may be formulated for intracellular protein delivery using methods known in the art including the use of anthrax lethal toxin for targeted delivery of protein into cells. (See WO 2014031861 A1; WO2008/076939; and WO 2001/21656, the contents of which are incorporated herein by reference in their entireties). Alternative approaches for targeted delivery of protein into cells are known in the art. (See, e.g., Weill et al., “A practical approach for intracellular protein delivery,” Cytotechnology. 20089 January; 56(1) 41-48; Walev et al., “Delivery of proteins into living cells by reversible membrane permeabilization with steptolysin-O,” PNAS, Mar. 13, 2001, vol. 98, no. 6, 3185-3190; Cronican et al., “Naturally supercharged human proteins (NSHPs),” Chemistry & Biology 18, 833-838, Jul. 29, 2011; Torchilin, “Intracellular deliver of protein and peptide therapeutics,” Drug Discovery Today: Technologies, Protein Therapeutics, 2009; M. Grdisa “The Delivery of Biologically Active (Therapeutic) Peptides and Proteins into Cells,” Cell-penetrating peptides (CPPS), Current Medicinal Chemistry, 2011. Vol. 18; Morris et al., “A Peptide Carrier for the Delivery of Biologically Active Proteins into Mammalian Cells: Application to the Delivery of Antibodies and Therapeutic Proteins,” Cell Biology, Volume 204, Part 20A, Chapter 2, 2006; Futami et al. “Intracellular delivery of proteins into mammalian living cells by polyethylenimine-cationization,” J Bioscience and Bioengineering, Vol. 99, Iss 2, February 2005 95-103; and Kurzawa et al., “PEP and CADY-mediated delivery of fluorescent peptides and proteins into living cells,” Biochimica et a Biophysica Acta (BBA)—Biomembranes Vol. 1798, Issue 12, December 2010 2274-2285).

Applications and Advantages of Disclosed Bacterial Toxins

Uses of the bacterial proteases disclosed herein include, but are not limited to: (a) uses as toxin components in bacterial toxin-based therapeutics for cancer and other diseases requiring killing of cells, including but not limited to immunotoxins, tetramer-toxins, bacterial delivery of toxins, and nanoparticles and others; (b) uses as reagents to treat cells to knock down Ras during biological research by direct delivery to cell cytosol by any method including chemical, mechanical, or biological strategies; (c) specific delivery by Protective antigen of this family of proteins to cells when fused to Lethal Factor N terminus as therapeutics; (d) specific delivery by Protective antigen of this family of proteins to cells when fused to Lethal Factor N terminus as a reagent during biological research; (e) treatment of biochemical reactions involving Ras to rapidly remove Ras from an in vitro reaction; (f) linkage of this family of proteins to an antibody or tetramer to create an immunotoxin specifically developed to delivery to cancerous cells or other conditions requiring killing of cells; and (g) delivery of this family of proteins to tumors or malignant cells by any strategy that delivers protein to cell for use a cancer therapeutic.

Some advantages of using the disclosed DUF5 protease or related proteases for inactivated Ras include, but are not limited to: (a) the DUF5 protease permanently modifies Ras by nicking Ras at a site essential for function, a modification which is not reversible as are other modifications; (b) the DUF5 protease exhibits specificity for isoforms of Ras including isoforms found in cancerous cells; (c) the DUF5 protease has a natural lack of structure in vitro and is thus amenable to easy transfer into cells by processes that require folding and unfolding; and (d) the DUF5 protease can be delivered to cells via fusion to anthrax toxin lethal factor (LF) in the presence of protective antigen (PA).

Pharmaceutical Compositions

The compositions disclosed herein may include pharmaceutical compositions comprising the presently disclosed bacterial toxins and formulated for administration to a subject in need thereof. Such compositions can be formulated and/or administered in dosages and by techniques well known to those skilled in the medical arts taking into consideration such factors as the age, sex, weight, and condition of the particular patient, and the route of administration.

The compositions may include pharmaceutical solutions comprising carriers, diluents, excipients, and surfactants, as known in the art. Further, the compositions may include preservatives (e.g., anti-microbial or anti-bacterial agents such as benzalkonium chloride). The compositions also may include buffering agents (e.g., in order to maintain the pH of the composition between 6.5 and 7.5).

The pharmaceutical compositions may be administered therapeutically. In therapeutic applications, the compositions are administered to a patient in an amount sufficient to elicit a therapeutic effect (e.g., a response which cures or at least partially arrests or slows symptoms and/or complications of disease (i.e., a “therapeutically effective dose”)).

### EXAMPLES

The following examples are illustrative and are not intended to limit the scope of the claimed subject matter.

### Example 1

### A Bacterial Toxin that is a Ras-Specific Protease

### BACKGROUND

Vibrio vulnificus is a motile, Gram-negative, opportunistic human pathogen capable of causing severe gastrointestinal and wound infections, both of which can be fatal. Two major virulence factors have been identified associated with increased death during intestinal infection: the secreted cytolytic/hemolysin pore-forming toxin encoded by _{Vv}hA [3] and the multifunctional autoprocessing RTX (MARTX_{Vv}) toxin encoded by gene rtxA1 [4-6]. However, results among different studies suggest that MARTX_{Vv} is the most significant virulence factor of V. vulnificus [7].

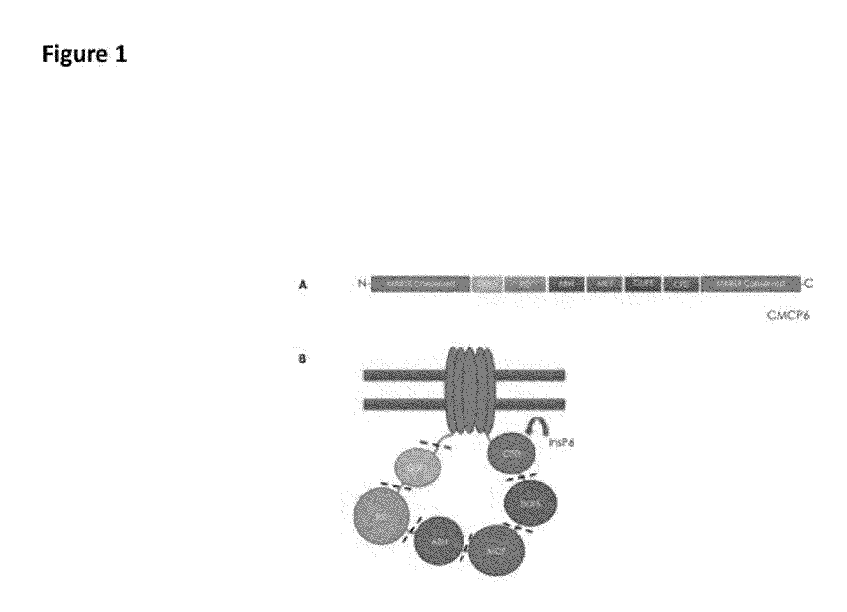

MARTX toxins are a recently described family of bacterial protein toxins originally characterized in Vibrio cholerae, but subsequently identified in many bacterial species [8][9, 10][6, 11][12]. These are large composite bacterial toxins that carry multiple effector domains that confer cellular toxicity after delivery by autoprocessing [9]. MARTX N- and C-termini repeats region are proposed to form a pore at the eukaryotic cell membrane for translocation of central “effector-domains” to the cytosol. Within the cytosol, the cysteine protease domain (CPD, covered by U.S. Pat. No. 8,257,946,B2) binds inositol hexakisphosphate and other inositol phosphate compounds, to initiate autoprocessing at leucine residues located in unstructured regions that link the “effector domains” [13-15]. The net result is release of the internal effector domains from the large protein toxin to the cytosol, where they are free to move throughout the cell to access cellular targets and to exert their toxic effects (FIG. 1).

Despite the sequence conservation of the repeats regions and the CPD in proteins produces by different bacteria, each bacterial MARTX toxin carries a distinct set of effector domains and thus a distinct array of potential cytotoxic activities [8, 9]. Further, different isolates of the same species can produce MARTX toxins that deliver distinct repertoire of effectors [6, 10, 12].

The first V. vulnificus MARTX toxin that was annotated was identified in the clinical isolates CMCP6 [8]. The central region of MARTX_{Vv} CMCP6 (NP_{—}759056.1) has five effector domains (FIG. 1A). Domain of unknown function in the first position (DUF1) has no functional homologs in the database, but is found also in MARTX toxins from Xenorhabdus bovienii and Xenorhabdus nematophila [8, 9] The second effector domain is Rho-inactivation domain (RID). This domain has been demonstrated to stimulate cell rounding by inactivating cellular Rho GTPases dependent upon a catalytic cysteine residue [16, 17] The third effector domain has homology to the αβ-hydrolase (ABH) family of enzymes [8, 9] and has recently been shown to have phospholipase activity (Agarwal S N and Satchell, manuscript in preparation). The fourth effector domain is 30% identical to a domain found within the Photorhabdus luminescens Makes Caterpillar Floppy (MCF) toxins [8, 9]. This domain is associated with induction of apoptosis (Agarwal S G and Satchell, manuscript in preparation).

DUF5 is the fifth effector domain in the toxin produced by V. vulnificus CMCP6 (DUF5_{Vv}), but absent in other isolates. Our group demonstrated that an in-frame genetic mutation on the chromosome of CMCP6 to remove DUF5_{Vv} from expressed MARTX_{Vv} toxin results in a 54-fold reduced virulence, compared with the isogenic strain CMCP6 that expresses the full-length toxin. In addition, a strain that naturally lacks this domain was at least 10-fold less virulent than CMCP6 [6]. Our interest in this domain was rooted in this identification that the presence of DUF5_{Vv} in the MARTX toxin of V. vulnificus is associated with the more highly virulent nature of V. vulnificus CMCP6 so we ventured to understand the molecular mechanism of action of this domain.

Details on the Discovery of the Catalytic Activity of DUF5_{Vv} and Related Proteins as Specific Endopeptidases for the Small GTPase Ras.

DUF5_{Vv} Represents a Family of DUF5-Like Proteins.

The DUF5 domain of the V. vulnificus CMCP6 MARTX toxin (DUF5_{Vv}) is found at amino acids G3579-L4089 based on Genbank sequence NP_{—}759056.1. Domains with similarity to DUF5_{Vv} are also found in MARTX toxins of at least 8 other bacterial species with amino acid identity varying from 43-98% identity.

DUF5_{Vv} homologs in other bacteria (% amino acid identity)

| Organisms with DUF5 homolog sequences | % Identity |
|---|---|
| Vibrio ordalii | 97.8 |
| Vibrio cholerae | 97.2 |
| Vibrio splendidus | 81.2 |
| Moritella dasanensis | 71.6 |
| Aeromonas salmonicida | 61.9 |
| Aeromonas hydrophila | 61.8 |
| PhotorabdusPhotorhabdus temperata | 58.8 |
| XenerhabdusXenorhabdus nematophila | 58.2 |
| Photorabdus luminescencesPhotorhabdus | 56.9 |
| luminescens | |
| PhotorabdusPhotorhabdus asymbiotica | 56.0 |
| Yersinia kristensenii | 42.5 |
| Pasteurella multocida | 24.4 |

The domain is found also in Photorhabdus sp. as a single domain hypothetical protein with 56-59% amino acid sequence identity to DUF5_{Vv} ([9] and search conducted for this document). DUF5_{Vv} also has 24% amino acid sequence identity to a portion of the Pasteurella multocida toxin (PMT) [9].

The solved structure of the C-terminus of PMT (PDB 2EBF) revealed three independent domains termed C1, C2, and C3 [18, 19]. The C3 domain is the catalytically active domain of PMT [20] and is not conserved in DUF5_{Vv}. The C1 domain in DUF5_{Vv}, PMT, and other bacterial toxins that have a homologous domain, has been shown to be a four helical bundled structure necessary for targeting toxin proteins to the cytosolic side of eukaryotic membranes [18, 21-24]. No function has been identified for the C2 domain of PMT. Transfection studies reveal this domain is not toxic when ectopically expressed in cells and bioinformatics comparing DUF5_{Vv} homologs suggest accumulated mutations in C2 may have rendered this domain inactive [39,40]. Thus, at the start of the project, there was no functional information regarding the activity of the C2 domain of DUF5_{Vv} or any of its protein homologues.

Structure of DUF5_{Vv}.

Recombinant DUF5_{Vv} (rDUF5_{Vv}; MARTX_{Vv} Q3596-L4089 based on sequence NP_{—}759056.1) was amplified from CMCP6 DNA and cloned into the expression vector pMCSG7 [25] to generate a fusion with a 6×HIS tag at its N-terminus for binding to a nickel column for affinity purification. The protein was expressed in E. coli and lysate prepared by sonication and centrifugation to recover the soluble fraction. rDUF5_{Vv} was purified from the lysate by affinity chromatography using pre-packed GE Biosciences HisTrap FF resin and then by size exclusion chromatography using a pre-packed GE Biosciences Superdex 5200 resin.

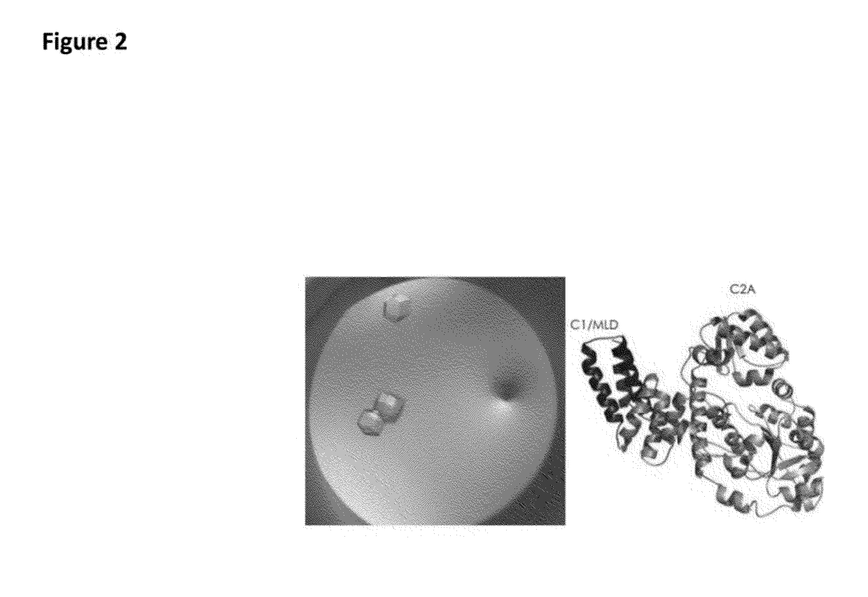

This rDUF5_{Vv} protein preparation was used for X-ray crystallography studies (FIG. 2). rDUF5_{Vv} structure was solved with an overall resolution of 3.4 Å. The overall structure of the protein aligns with the previously determined structure of PMT C1/C2 domains (RMSD=2.75) despite the fact that the two proteins share only 24% amino acid identity. The solved structure revealed that rDUF5_{Vv} as predicted by secondary structure alignment is composed also of C1 (aa 3579-3669) and C2 domains (amino acids 3670-4089). The C2 domain could likewise be bisected into two subdomains: C2A (amino acids 3669-3855) and C2B (amino acids 3856-4089). Bioinformatics studies had also predicted two subdomains for C2 but predicted the active catalytic activity would be focused on C2B [39,40].

The C2A Subdomain is the Cytotoxic Portion of DUF5_{Vv}.

To probe whether DUF5_{Vv} has cytotoxic or cytopathic activity, the DNA sequence from V. vulnificus CMCP6 corresponding to DUF5_{Vv} (amino acids 3579-4089) was amplified by PCR, cloned in the pEGFP-N3 (Clontech Laboratories Inc.) to generate a fusion with green fluorescent protein gene (egfp) and the resulting plasmid chemically transfected into HeLa cells. These studies showed rounding of cells that were expressing the EGFP fusion protein, but not control cells that were expressing EGFP alone. C2 also induced cell rounding when expressed in the eukaryotic yeast Saccharomyces cerevisiae. The minimal portion of DUF5_{Vv} that demonstrated the cytopathic activity in HeLa cells was linked to the C2A domain by deletion analysis.

To further demonstrate that DUF5_{Vv} is toxic to cells, the DNA sequence from V. vulnificus CMCP6 corresponding to DUF5_{Vv} (amino acids 3579-4089) was amplified by PCR, cloned into pRT24 (a modified version of pABII [42]) to generate a fusion with 6×His-tagged anthrax toxin lethal factor N-terminus (LF_{N}) at the N-terminus. This protein LF_{N}DUF5_{Vv} can be delivered to the cytosol of cells by adding the purified protein to the cell culture media along with anthrax toxin protective antigen (PA), which is purified separately as a 6×His-tagged protein. The PA portion of the bipartitite anthrax toxin associates with the LF_{N} portion of the fusion protein and LF_{N}DUF5_{Vv} is then translocated into the cell cytosol by PA, allowing for delivery of DUF5_{Vv} to the cell cytosol independent of the remainder of the MARTX toxin. This intoxication system has been used for the study of many bacterial toxins and other proteins [16, 41-44]. Several embodiments of the Lethal Factor/Protective antigen translocation system have been described (WO 2014031861 A1, WO 2001/21656 and WO2008/076939).

Cells intoxicated with LF_{N}DUF5_{Vv} in combination with PA exhibited cell rounding, including HeLa cervical carcionoma cells, J774 macrophages, 293T fibroblasts, etc. Cells were not rounded by LF_{N}DUF5_{Vv} in the absence of PA or by PA in combination with purified LF_{N} alone (without the DUF5_{Vv}). The minimal portion of DUF5_{Vv} essential for cytoxicity when delivered by LF_{N} was mapped to MARTX_{Vv} G3579-T3855 corresponding to the C1 domain plus C2A, as C1 is essential for toxin to reach the membrane after delivery though the PA pore.

Similarly, it was found that cells treated with LF_{N} fused to the DUF5 domain from the Aeromonas hydrophila MARTX toxin (aa 3041-3575 based on sequence strain 7966, from ATCC, GI: 117618727) also demonstrated cell rounding when delivered to cells and only when in combination with PA. Thus, despite having only 62% amino acid identity, these proteins seem to share a toxic mechanism.

Discovery of DUF5_{Vv} Targeting Ras.

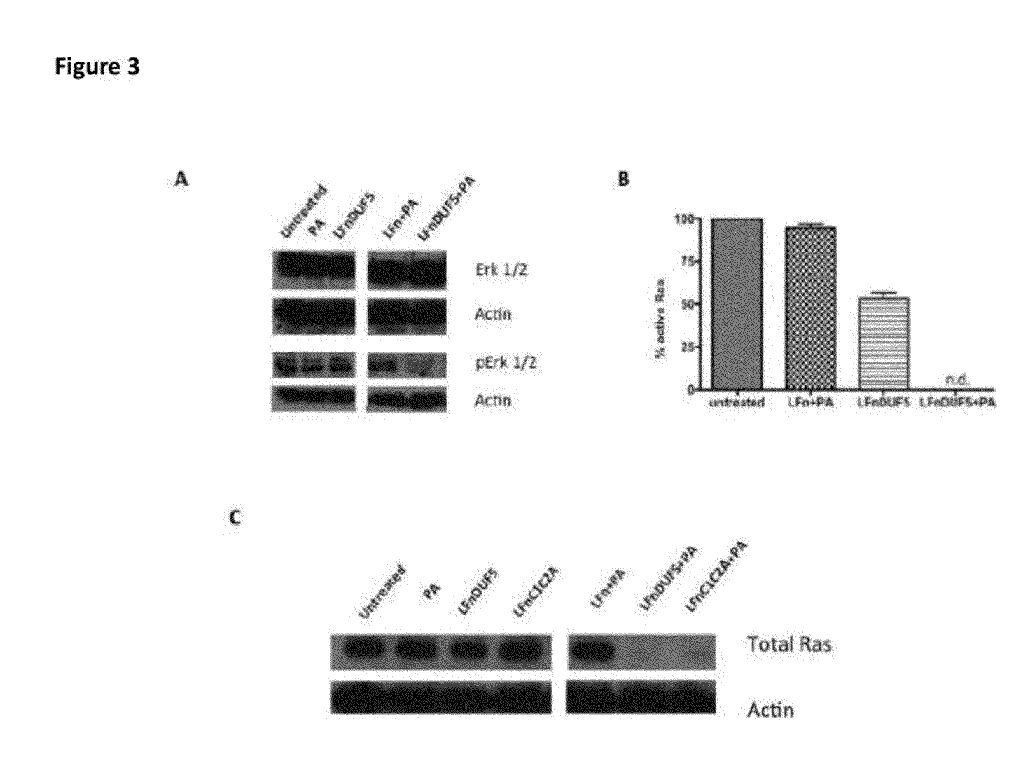

To further investigate the cytopathic function of DUF5_{Vv}, a screen was conducted for suppressors in yeast that would permit growth of yeast when DUF5_{Vv} C2 subdomain was ectopically expressed. This screen revealed >100 suppressor mutations that mapped to a plethora of cellular signaling pathways, enriched in pathways linked to cellular stress responses. Based on this finding, we investigated if the major transcription factor activated under conditions of cell stress in human epithelial cells—ERK1/2—would be affected by DUF5_{Vv} accounting for the observed wide variety of downstream effects in yeast. Cells intoxicated with LF_{N}DUF5_{Vv} in the presence of PA were found to have reduced levels of phosphorylated ERK1/2 (pERK1/2) (FIG. 3A, lower panels), despite having no difference in total levels of ERK1/2 (FIG. 3A, upper panels). This result demonstrated that DUF5_{Vv} does suppress the stress response pathways in cells controlled by ERK1/2.

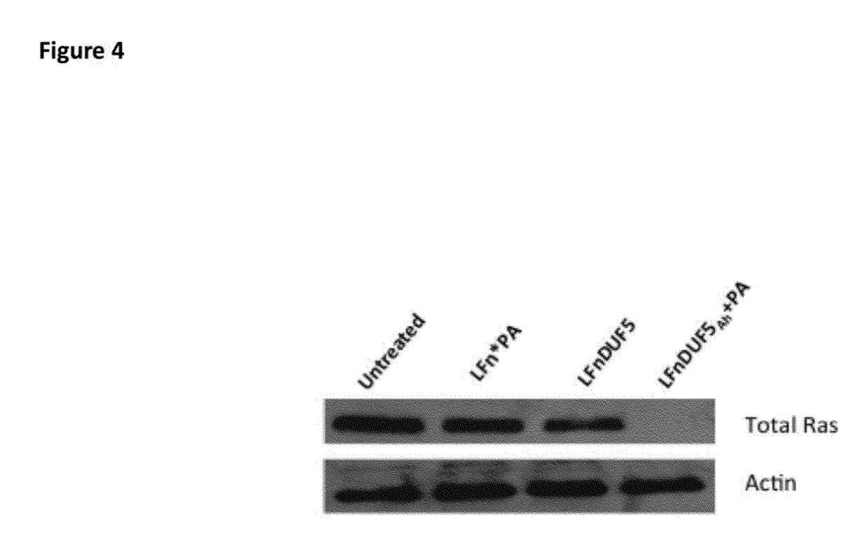

The relative concentration of active pERK1/2 in the cell is normally regulated by the Ras-Raf-MEK-ERK signaling cascade. At the top of the cascade, Ras is activated by conversion from an inactive, GDP-bound state to an active, GTP-bound state [26, 27]. As pERK1/2 levels were reduced, this led us to test if the activation state of Ras in LF_{N}DUF5_{Vv} intoxicated HeLa cells was affected. The G-LISA activation assay, commercially available from Cytoskeleton, Inc., specifically detects the GTP-bound activated form of all Ras isoforms dependent upon the final detection of Ras by the monoclonal antibody Ras10. This assay detected no active Ras-GTP in intoxicated cells (FIG. 3B). Western blotting, using the Ras10 monoclonal antibody to detect the total amount of Ras within the HeLa whole cell lysate, showed that Ras was absent from lysates prepared from cells intoxicated with LF_{N}DUF5_{Vv} or LF_{N}C1C2A exclusively when incubated in the presence of PA (FIG. 3C). HeLa cells intoxicated for shorter time points than 24 hours revealed that loss of Ras detectable by the Ras10 monoclonal antibody occurred as early as 20 minutes from the time of exposure of cells to LF_{N}DUF5_{Vv} in the presence of PA (FIG. 4).

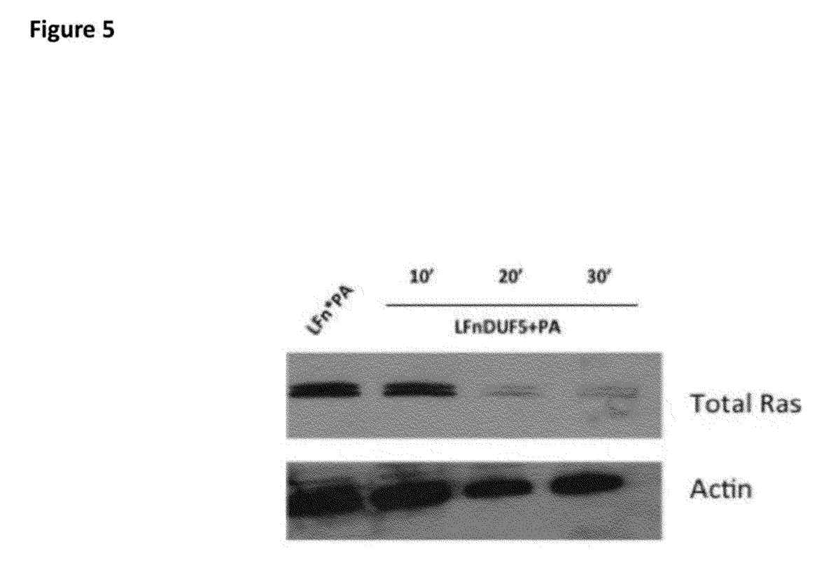

Following a strategy similar to DUF5_{Vv}, we demonstrated that the HeLa cells intoxication with an LF_{N} fusion of DUF5 from MARTX (LF_{N}DUF_{Ah}) (amino acid 3069-3570) also causes cell rounding after 24 hours. Western blot analysis was used to detect the total amount of Ras into the HeLa cell lysates demonstrated that Ras was undetectable into the HeLa cell lysate after intoxication with DUF5 (FIG. 5), similar to DUF5_{Vv} intoxicated cells. This demonstrates that DUF_{Ah} has potentially a similar mechanism as DUF5_{Vv}.

Ras is Truncated in DUF5_{Vv} Intoxicated Cells.

The failure to detect all forms of Ras by the Ras10 antibody due to intoxication of cells by DUF5_{Vv} and DUF5 was initially thought to be due to a covalent modification of Ras that disturbed the detection of Ras by the antibody. Other bacterial toxins are known to target Ras in this way. These include Pseudomonas aeruginosa ExoS that can ADP-ribosylate Ras (U.S. Pat. No. 5,599,665 A), but also modifies up to 20 other cellular proteins [28-30]. Clostridium sordelli TcsL or Clostridium perfringens TpeL are monoglucosyltransferases that can UDP-glucosylates Ras (Patent EP 0877622 B1), but also modifies many other small GTPase proteins like Rac [31-33][45]. Similarly, C. difficile TcdA and TcdB show UDP-glucosylation modification of many small GTPases including Ras [45]. The recently revealed lack of specificity of these proteins has made them poor candidates for toxins that would attack Ras when developed as toxin therapeutics.

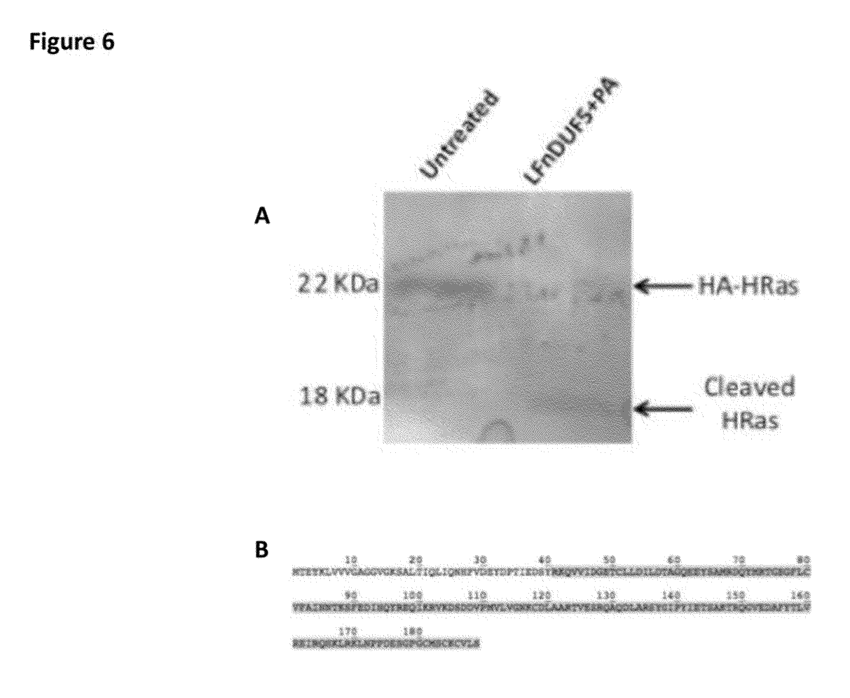

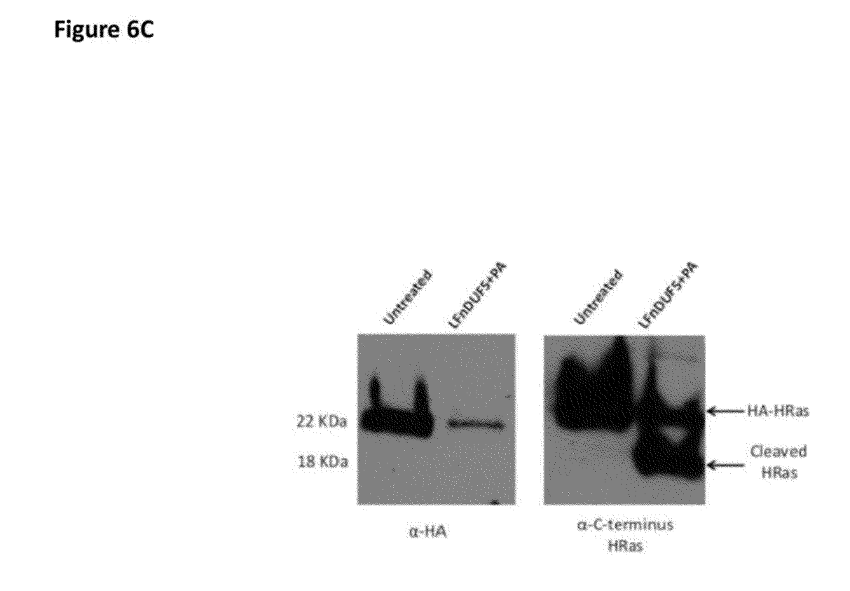

As a first step to identify the modification in Ras that prevented detection with the Ras10 monoclonal antibody in cells intoxicated with DUF5_{Vv}, HeLa cells were transiently transfected to express the H-Ras isoform with a hemagglutinin (HA) tag (sequence YPYDVPDYA, SEQ ID NO:29) fused at the N-terminus for detection by the HA peptide monoclonal antibody. These cells were intoxicated for 24 hr LF_{N}DUF5_{Vv} in the presence of PA after which the HA-HRas protein was immunoprecipitated from cell lysate with using agarose beads conjugated with to the anti-HA peptide monoclonal antibody. The proteins specifically bound to the beads were eluted 3 M sodium thiocyanate solution and separated on an SDS-polyacrylamide gel. Coomassie brilliant blue staining of the gel revealed a 22 kDa protein band corresponding to HA-HRas for the unintoxicated Hela cells sample. However, for the intoxicated cells, an 18 kDa protein band was evident (FIG. 6A). Subsequent analysis of the trypsin-digested excised protein band by mass spectrometry revealed that the 18 kDa band was HRas, but absent the N-terminus (FIG. 6B). Western blot analysis, using anti-HA peptide monoclonal antibody and an anti-HRas polyclonal antibody specific for the C-terminus, confirmed that the 18 kDa band is HRas but truncated to remove the HA tag and the N-terminus of HRas (FIG. 6C). This result indicated that DUF5_{Vv} is either an endopeptidase or activates a previously unknown host cell endopeptidase that targets the N-terminus of H-Ras.

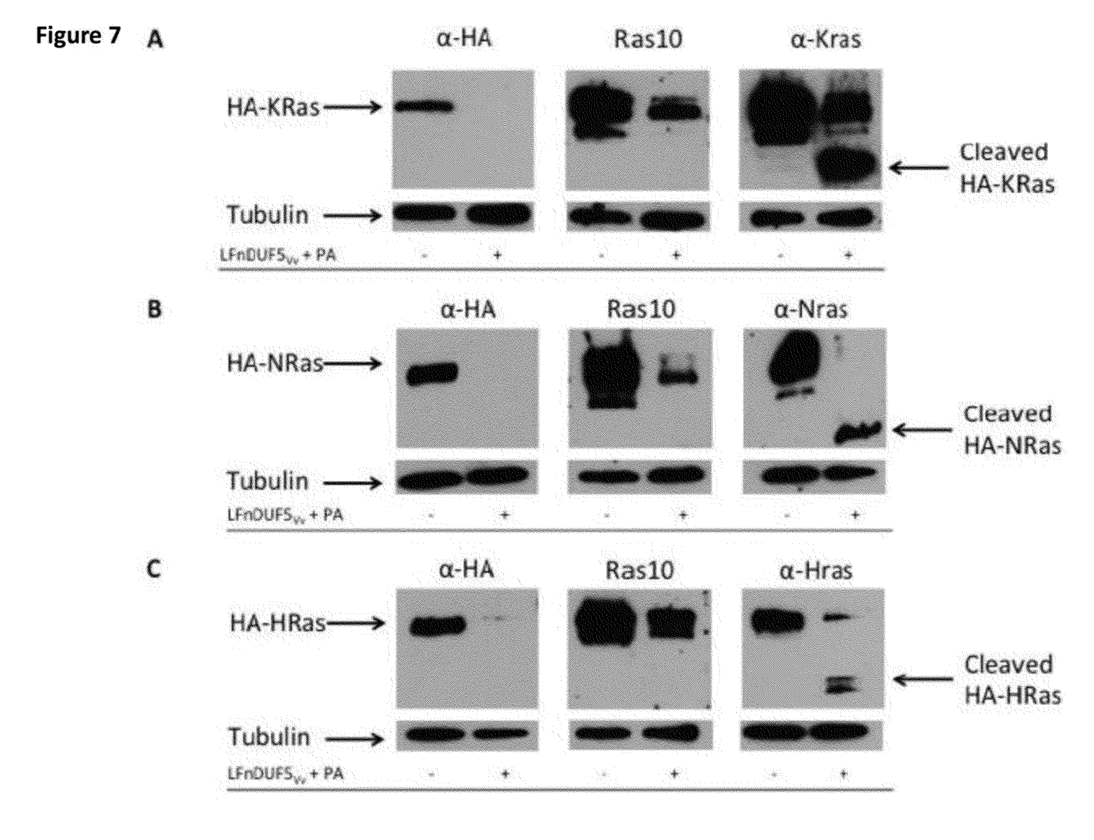

In addition, cells were transfected with plasmids to express HA-tagged versions of KRas, NRas, and HRas. All 3 isoforms of Ras were susceptible to cleavage in vivo resulting in truncated proteins that are not detected by the anti-HA antibody, but are detectable by isoform specific antibodies that recognize the unique C-terminus of each of the isoforms (FIG. 7).

DUF5_{Vv} is Itself an Endopeptidase that Targets Ras.

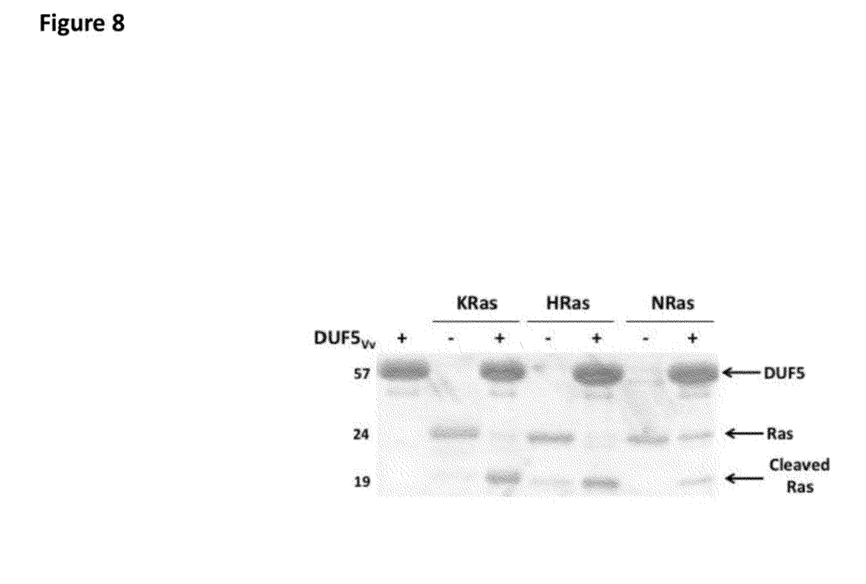

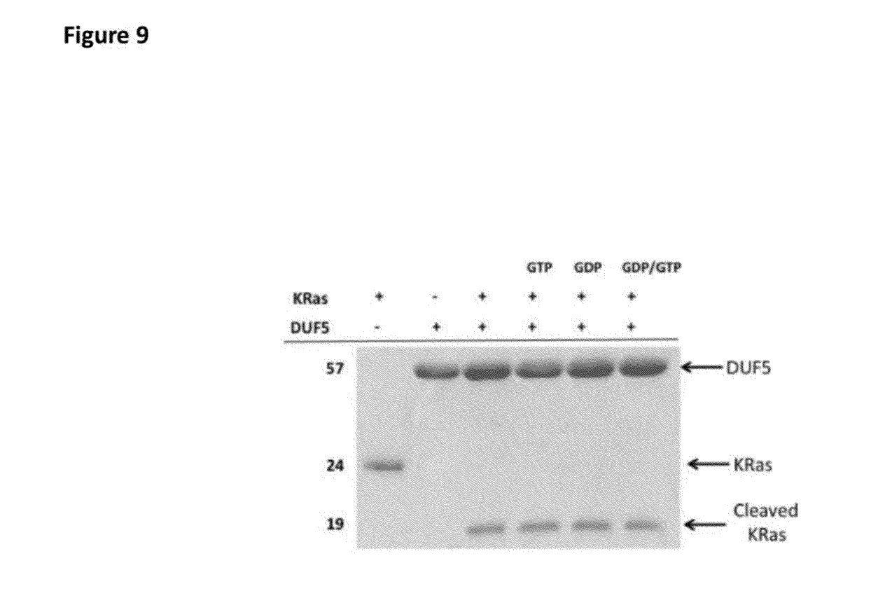

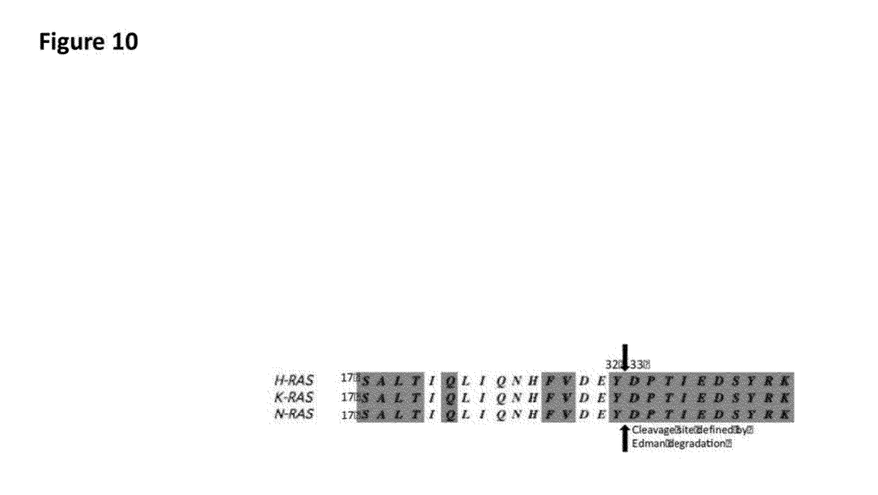

To test if DUF5_{Vv} is itself an endopeptidase that targets Ras isoforms rather than an activator of a host protease, gene sequences for KRas (KRas4B NP_{—}004976.2), HRas (NP_{—}001123914.1) and NRas (NP_{—}002515.1) were cloned into pMCSG7 vector for E. coli expression with a 6×His tag at the N-terminus for nickel affinity purification. Recombinant rKRas and rHRas were purified from E. coli cell lysates using a pre-packed GE Biosciences His Trap FF column for single step NiNTA affinity chromatography. Recombinant NRas (rNRas) was expressed in inclusion bodies. The protein was therefore recovered from the insoluble fraction by suspension in buffer containing urea, purified by single step purification with NiNTA, and then rNRas refolded in the presence of excess GDP. rKRas, rHRas and rNRas were tested in vitro as substrate for rDUF5_{Vv} (previously purified for crystallography studies described above) for an endopeptidase assay. The reaction products were analyzed by SDS-PAGE showed the cleavage of rKRas, rHRas, and rNRas by rDUF5_{Vv}. (FIG. 8). The cleavage of KRas was shown to occur regardless of the presence of guanosine nucleosides (FIG. 9). The cleavage of rNRas was less efficient compared to rKRas and rHRas, but this was likely due to the requirement to refold the protein resulting in a mixed pool of proper and improper folded substrate rather than a preference for substrate as there was no difference in substrate specificity in vivo (FIG. 7). Cleavage products for each reaction were analyzed by Edman degradation for N-terminal sequencing. The results revealed that DUF5 protein specifically cleaves KRas, HRas and NRas between residues Y32 and D33. (FIG. 10). These two residues are in the middle of Switch I region of KRas. Overall, these results confirm that rDUF5_{Vv} is itself an endopeptidase able to cleave all common isoforms of Ras in vitro without host cell cofactors.

DUF5 Endopeptidase Activity in Aeromonas hydrophila and Photorhabdus asymbiotica.

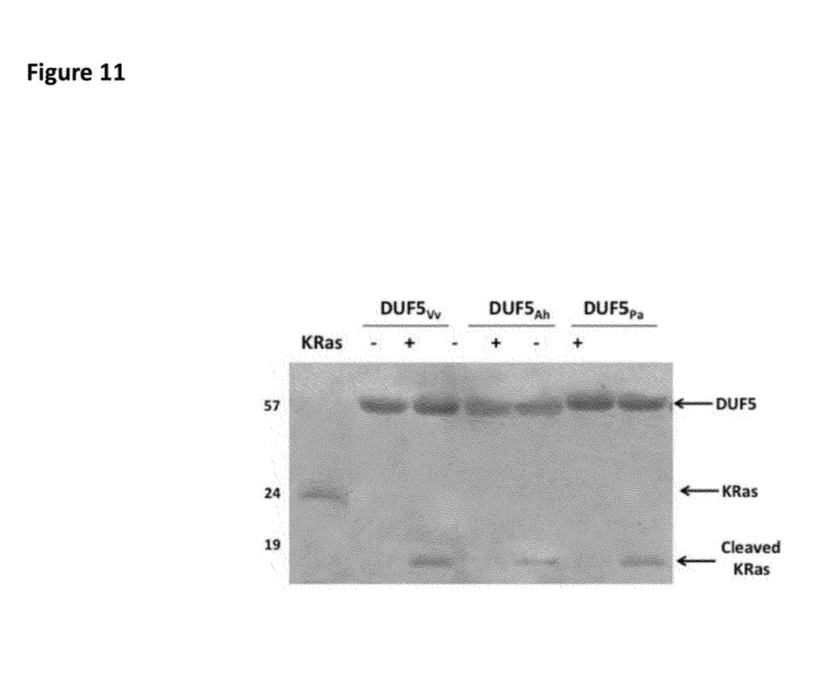

As detailed above, DUF5 from the A. hydrophila MARTX toxin effector domain is 62% identical to DUF5_{Vv} and induced similar phenotypes as DUF5_{Vv} when delivered to cells in vivo. Gene sequences for DUF5 were cloned into pMCSG7 vector for E. coli expression and purified similarly to rDUF5_{Vv}. The recombinant protein rDUF5 was able to cleave rKRas in the in vitro reaction (FIG. 11) demonstrating that the same domain from a different MARTX toxin is also an endopeptidase for Ras. This result indicates these are representative members of the larger family of MARTX effectors from at least 8 MARTX toxin and that all DUF5 domains from MARTX toxins will have this activity.

In addition to its presence in MARTX toxins, a hypothetical protein of Photorhabdus spp. (i.e. P. asymbiotica PAT3383 and P. luminescens Plu2400) has 56-59% similarity to DUF5_{Vv}. In Photorhabdus spp., this hypothetical proteins is not linked to a MARTX toxin but instead is found as a stand-alone gene that encodes a 542-568 aa hypothetical protein. Recombinant PAT3383 (here known as DUF5_{Pa}) was also successfully purified and shown to also cleave rKRas. N-terminal sequencing by Edman degradation of products excised from gel showed that all three DUF5 (DUF5_{Vv}, DUF5 and DUF5_{Pa}) cleave KRas between Y32 and D33. To our knowledge, none of the several DUF5 homologs identified has ever been characterized for its intrinsic function. DUF5 has been recently studied for its thermodynamic properties in the context on MARTX toxin unfolding and translocation [34].

DUF5_{Vv} Endopeptidase is Specific for Ras and does not Process Representative Members of Other Small GTPases.

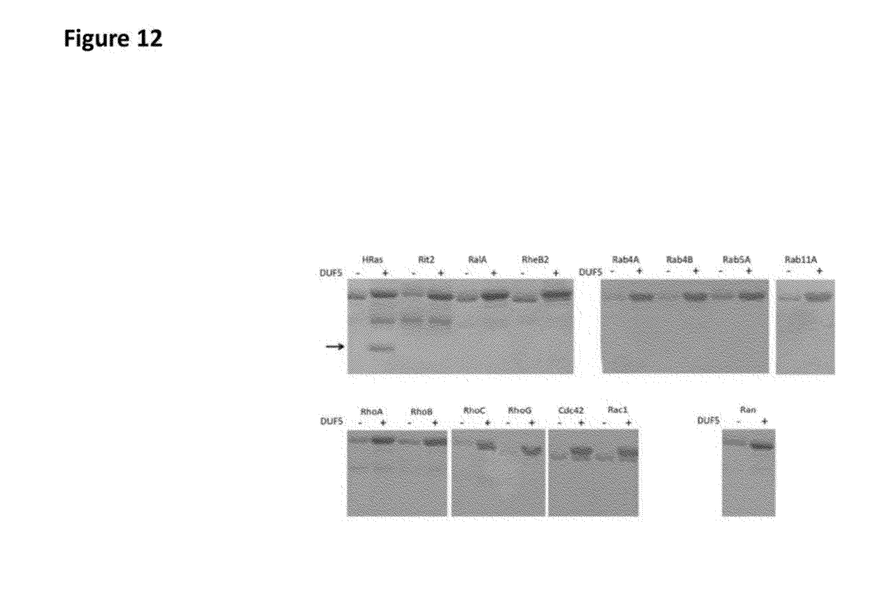

DUF5_{Vv} specificity was further tested by examining cleavage of representative members of small GTPase family. Recombinant proteins for other fused Ras family members (Rit2, RalA and RheB2) and small GTPase from other Ras superfamily groups: Rab (Rab4A, Rab4B, Rab5A and Rab11A), Rho (RhoA, RhoB, RhoC, RhoG, Cdc42 and Rac1) and Ran. Each protein was individually expressed in E. coli fused to glutathione-S-transferase for purification on glutathione agarose. Cloning, expression and purification condition of this rGTPase library was previously reported [35]. The in vitro cleavage assay was performed incubating each purified rGST-GTPase with rDUF5_{Vv}, rGST-HRas was used as positive control to demonstrate that the presence of GST does not interfere with the cleavage assay. The reaction products, analyzed by SDS-PAGE, showed that DUF5_{Vv} could cleave only HRas. None of the other GTPase was cleaved by DUF5_{Vv} (FIG. 12). The overall results demonstrate that DUF5_{Vv} is a novel Ras endopetidase for, which cleaves specifically KRas, HRas and NRas.

DUF5 Endopeptidase Activity and Mutant KRas.

In this application, we propose that the Ras-directed endopeptidase activity of DUF5_{Vv} and homologous proteins with similar activity can be directed toward treatment of cancers. As DUF5_{Vv} targets normal Ras to compromise the cell, it can be utilized in a vast array of cancers. However, a particular focus of this work could be to target cancers that result from mutation of Ras itself. To achieve this, cells that have Ras with amino acid substitutions must be shown to be susceptible to DUF5_{Vv}.

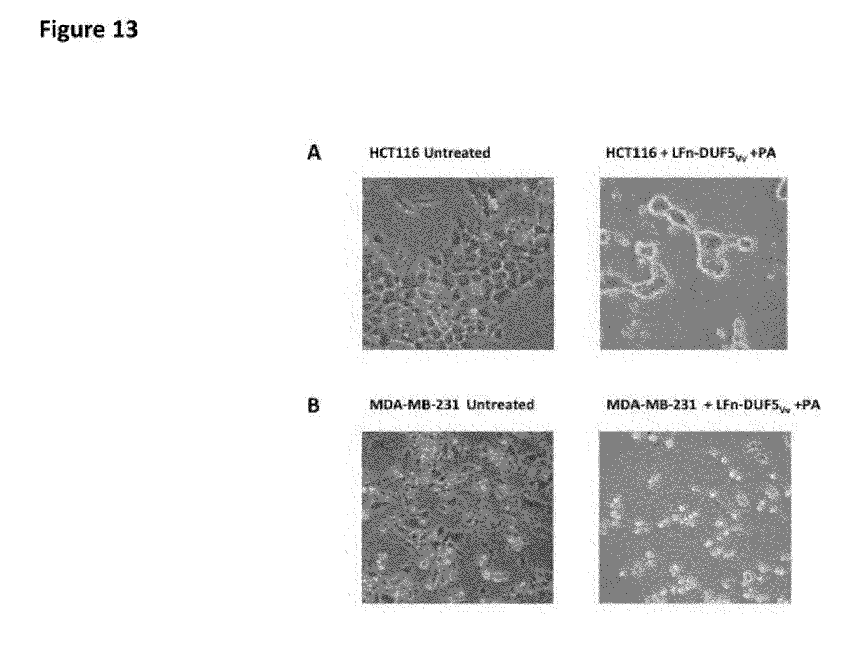

The cytotoxicity of DUF5_{Vv} was tested in colorectal cancer cells (HCT116) and in breast cancer cells (MDA-MB-231). These two cells lines express, respectively, mutant KRas G12V and G13D. A dramatically morphology change was observed for HCT116 after 24 hours of intoxication with LF_{N}DUF5_{Vv} in the presence of PA (FIG. 13A). The intoxicated cells showed a reduction in the number of cells and cell enlargement, suggesting swelling. In addition, the cells were observed to detach from the dish surface. MDA-MB-231 cells intoxicated with LF_{N}DUF5_{Vv} for 24 hours showed a more “typical” cell rounding phenotype, similar to that previously observed in HeLa cells (FIG. 13B). With these experiments, we demonstrated the toxicity of DUF5_{Vv} for cancer cells that are expressing mutant forms of KRas.

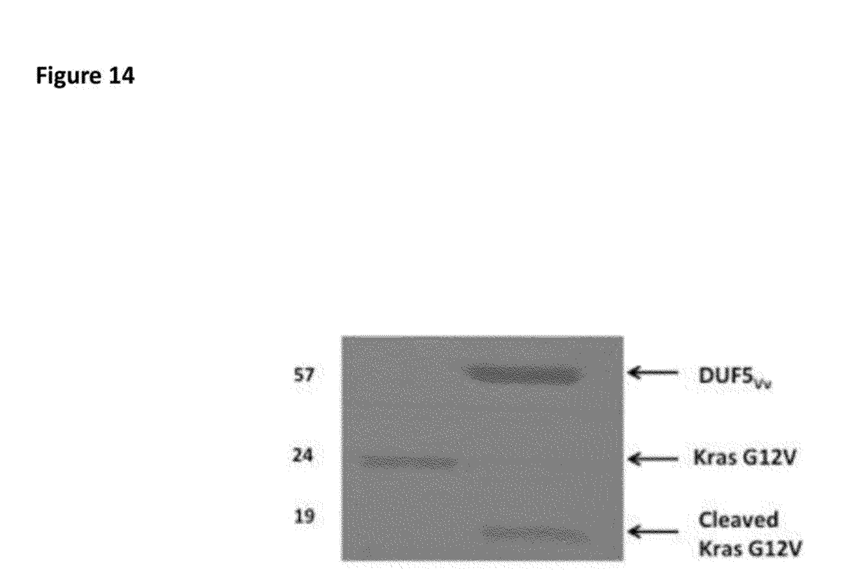

As further evidence of its applicability to treatment of Ras cancers, recombinant mutant KRas G12V was cloned into pMCSG7 and expressed in E. coli. The purified rKRas G12V was incubated with rDUF5_{Vv} to check its cleavability in vitro. The reaction products, analyzed on SDS-PAGE, showed that DUF5_{Vv} is still able to cleave mutant KRas (G12V) (FIG. 14).

Benefits Over Other Technologies.

Many bacterial toxins have been proposed for use in chemotherapy. Toxins that destroy the membrane, such as pore forming toxins have the potential to induce inflammation resulting in severe side effects. The advantage of this toxin over others is that it works from inside the cell to block normal cell survival pathways, thereby inducing loss of proliferation and normal non-inflammatory cell death.

Unlike toxins that target such processes as protein translation, this toxin directly targets a central regulatory pathway that is normal altered in cancer cells to promote cell survival and is thus key to the survival of the cancer itself. Ras cancers are among the most difficult to treat cancers due to the mutations in Ras. By directly targeting Ras in these cells, we can remove the protein that is driving the survival of the cancer.

A tripping point for some toxins (except those that form pores from the outside) is the ability to deliver to the cell cytosol where they can access target. We demonstrate that the DUF5 protein can be easily delivered to cells in an active form by the LF_{N}-PA delivery system. This system has already been modified to directly target cancer cells. A problem with the LF_{N}-PA delivery system, is that it is selective to translocate proteins that can rapidly unfold and spontaneously refold. We showed that this protein is able to cleave all molecules of Ras in cells at less then 30 minute after exposure indicating rapid translocation and delivery of active protein via the PA pore. Other delivery strategies will also require self-folding. We were able to purify this protein to homogeneity for the purpose of crystallography indicating that despite its plasticity, it is a stable protein for storage in vitro.

The specificity for Ras is also a benefit. Unlike other toxins that target Ras, this protein does not as yet show any specificity outside of HRas, NRas, and KRas. It does not target other small GTPases, which is the case for the Clostridial toxins TcsL, Tpel, TcdA, and TcdB. It does not show evidence of having cellular substrates in a wide range of protein families such as Pseudomonas Exotoxin A. Finally, these other proteins covalently modify the substrate, which there is some evidence is reversible. By contrast, DUF5 irreversibly cleaves the Ras proteins and thus cannot be reversed by the cell. For diversity of immunogenicity and increasing efficacy and activity are at least three different family members that share this activity and these are representative of the families across a wide range of bacteria species.

### REFERENCES FOR EXAMPLE 1

1. Antignani A, Fitzgerald D: Immunotoxins: the role of the toxin. Toxins 2013, 5(8):1486-1502.
2. Shapira A, Benhar I: Toxin-based therapeutic approaches. Toxins 2010, 2(11):2519-2583.
3. Fan J J, Shao C P, Ho Y C, Yu C K, Hor L I: Isolation and characterization of a Vibrio vulnificus mutant deficient in both extracellular metalloprotease and cytolysin. Infection and immunity 2001, 69(9):5943-5948.
4. Chung K J, Cho E J, Kim M K, Kim Y R, Kim S H, Yang H Y, Chung K C, Lee S E, Rhee J H, Choy H E et al: RtxA1-induced expression of the small GTPase Rac2 plays a key role in the pathogenicity of Vibrio vulnificus. The Journal of infectious diseases 2010, 201(1):97-105.
5. Kim Y R, Lee S E, Kook H, Yeom J A, Na H S, Kim S Y, Chung S S, Choy H E, Rhee J H: Vibrio vulnificus RTX toxin kills host cells only after contact of the bacteria with host cells. Cellular microbiology 2008, 10(4):848-862.
6. Kwak J S, Jeong H G, Satchell K J: Vibrio vulnificus rtxA1 gene recombination generates toxin variants with altered potency during intestinal infection. Proceedings of the National Academy of Sciences of the United States of America 2011, 108(4):1645-1650.
7. Jeong H G, Satchell K J: Additive function of Vibrio vulnificus MARTX(_{Vv}) and _{Vv}hA cytolysins promotes rapid growth and epithelial tissue necrosis during intestinal infection. PLoS pathogens 2012, 8(3): e1002581.
8. Satchell K J: MARTX, multifunctional autoprocessing repeats-in-toxin toxins. Infection and immunity 2007, 75(11):5079-5084.
9. Satchell K J: Structure and function of MARTX toxins and other large repetitive RTX proteins. Annual review of microbiology 2011, 65:71-90.
10. Roig F. J. G-C, F. and Amaro C.: Domain organization and evolution of multifunctional autoprocessing repeats-in-toxin (MARTX) toxin in Vibrio vulnificus. Appl Environ Microbiol 2011, 77:657-668.
11. J. DJaSK: Analysis of Vibrio cholerae genome sequences reveals unique rtxA variants in environmental strains and an rtxA-null mutation in recent altered El Tor isolates. mBio 2013, 4:e00624-00612.
12. Ziolo K J, Jeong H G, Kwak J S, Yang S, Lavker R M, Satchell K J: Vibrio vulnificus biotype 3 multifunctional autoprocessing RTX toxin is an adenylate cyclase toxin essential for virulence in mice. Infection and immunity 2014, 82(5):2148-2157.
13. Egerer M, Satchell K J: Inositol hexakisphosphate-induced autoprocessing of large bacterial protein toxins PLoS pathogens 2010, 6(7):e1000942.
14. Prochazkova K, Satchell K J: Structure-function analysis of inositol hexakisphosphate-induced autoprocessing of the Vibrio cholerae multifunctional autoprocessing RTX toxin. The Journal of biological chemistry 2008, 283(35):23656-23664.
15. Prochazkova K, Shuvalova L A, Minasov G, Voburka Z, Anderson W F, Satchell K J: Structural and molecular mechanism for autoprocessing of MARTX toxin of Vibrio cholerae at multiple sites. The Journal of biological chemistry 2009, 284(39):26557-26568.
16. Sheahan K L, Satchell K J: Inactivation of small Rho GTPases by the multifunctional RTX toxin from Vibrio cholerae. Cellular microbiology 2007, 9(5):1324-1335.
17. Ahrens S. GBaSKJ: Identification of small Rho GTPases by the multifunctional RTX toxin from Vibrio cholerae. The Journal of biological chemistry 2013, 288:1397-1408.
18. Kamitani S, Kitadokoro K, Miyazawa M, Toshima H, Fukui A, Abe H, Miyake M, Horiguchi Y: Characterization of the membrane-targeting C1 domain in Pasteurella multocida toxin. The Journal of biological chemistry 2010, 285(33):25467-25475.
19. Kitadokoro K, Kamitani S, Miyazawa M, Hanajima-Ozawa M, Fukui A, Miyake M, Horiguchi Y: Crystal structures reveal a thiol protease-like catalytic triad in the C-terminal region of Pasteurella multocida toxin. Proceedings of the National Academy of Sciences of the United States of America 2007, 104(12):5139-5144.
20. Orth J H, Preuss I, Fester I, Schlosser A, Wilson B A, Aktories K: Pasteurella multocida toxin activation of heterotrimeric G proteins by deamidation. Proceedings of the National Academy of Sciences of the United States of America 2009, 106(17):7179-7184.
21. Geissler B, Tungekar R, Satchell K J: Identification of a conserved membrane localization domain within numerous large bacterial protein toxins. Proceedings of the National Academy of Sciences of the U.S. Pat. No. 2,010,107(12):5581-5586.\
22. Geissler B, Ahrens S, Satchell K J: Plasma membrane association of three classes of bacterial toxins is mediated by a basic-hydrophobic motif. Cellular microbiology 2012, 14(2):286-298.
23. Brothers M C, Geissler B., Hisao G. S., Satchell K. J., Wilson B. A. and Rienstra C. M.: Backbone and side-chain resonance assignments of the membrane localization domain from Pasteurella multocida toxin. Biomolecular NMR assignments 2013.
24. Brothers M C, Geissler B, Hisao G S, Wilson B A, Satchell K J, Rienstra C M: Backbone and side-chain assignments of an effector membrane localization domain from Vibrio vulnificus MARTX toxin. Biomolecular NMR assignments 2013.
25. Stols L, Gu M, Dieckman L, Raffen R, Collart F R, Donnelly M I: A new vector for high-throughput, ligation-independent cloning encoding a tobacco etch virus protease cleavage site. Protein expression and purification 2002, 25(1):8-15.
26. Chang F, Steelman L S, Lee J T, Shelton J G, Navolanic P M, Blalock W L, Franklin R A, McCubrey J A: Signal transduction mediated by the Ras/Raf/MEK/ERK pathway from cytokine receptors to transcription factors: potential targeting for therapeutic intervention. Leukemia 2003, 17(7):1263-1293.
27. Steelman L S, Franklin R A, Abrams S L, Chappell W, Kempf C R, Basecke J, Stivala F, Donia M, Fagone P, Nicoletti F et al: Roles of the Ras/Raf/MEK/ERK pathway in leukemia therapy. Leukemia 2011, 25(7):1080-1094.
28. Ganesan A K, Vincent T S, Olson J C, Barbieri J T: Pseudomonas aeruginosa exoenzyme S disrupts Ras-mediated signal transduction by inhibiting guanine nucleotide exchange factor-catalyzed nucleotide exchange. The Journal of biological chemistry 1999, 274(31):21823-21829.
29. Maresso A W, Baldwin M R, Barbieri J T: Ezrin/radixin/moesin proteins are high affinity targets for ADP-ribosylation by Pseudomonas aeruginosa ExoS. The Journal of biological chemistry 2004, 279(37):38402-38408.
30. Simon N C, Barbieri J T: Exoenzyme S ADP-ribosylates Rab5 effector sites to uncouple intracellular trafficking. Infection and immunity 2014, 82(1):21-28.
31. Just I, Selzer J, Hofmann F, Green G A, Aktories K: Inactivation of Ras by Clostridium sordellii lethal toxin-catalyzed glucosylation. The Journal of biological chemistry 1996, 271(17):10149-10153.
32. Guttenberg G, Hornei S, Jank T, Schwan C, Lu W, Einsle O, Papatheodorou P, Aktories K: Molecular characteristics of Clostridium perfringens TpeL toxin and consequences of mono-O-GlcNAcylation of Ras in living cells. The Journal of biological chemistry 2012, 287(30):24929-24940.
33. Nagahama M, Ohkubo A, Oda M, Kobayashi K, Amimoto K, Miyamoto K, Sakurai J: Clostridium perfringens TpeL glycosylates the Rac and Ras subfamily proteins. Infection and immunity 2011, 79(2):905-910.
34. Kudryashova E, Heisler D, Zywiec A, Kudryashov D S: Thermodynamic properties of the effector domains of MARTX toxins suggest their unfolding for translocation across the host membrane. Molecular microbiology 2014.
35. Mattoo S, Durrant E, Chen M J, Xiao J, Lazar C S, Manning G, Dixon J E, Worby C A: Comparative analysis of Histophilus somni immunoglobulin-binding protein A (IbpA) with other fic domain-containing enzymes reveals differences in substrate and nucleotide specificities. The Journal of biological chemistry 2011, 286(37):32834-32842.
36. Malumbres M, Barbacid M: RAS oncogenes: the first 30 years. Nature reviews Cancer 2003, 3(6):459-465.
37. Shimizu K, Goldfarb M, Perucho M, Wigler M: Isolation and preliminary characterization of the transforming gene of a human neuroblastoma cell line. Proceedings of the National Academy of Sciences of the United States of America 1983, 80(2):383-387.
38. Downward J: Targeting RAS signalling pathways in cancer therapy. Nature reviews Cancer 2003, 3(1):11-22.
39. Bazan J, Macdonald B, He X: The TIKI/TraB/PrgY family: a common protease fold for cell signaling from bacteria to metazoa? Developmental Cell 2013; 25(3):225-227.
40. Sanchez-Pulido L, Ponting C: Tiki, at the head of a new superfamily of enzymes. Bioinformatics 2013; 29(19):2371-2374.
41. Cordero C, Kudryahov D, Reisler E, Satchell K: The actin crosslinking domain of the Vibrio cholerae RTX toxin directly catalyzes the covalent cross-linking of actin. The Journal of Biological Chemistry 2006; 283(43)32366-32374.
42. Spyres L, Qa'Dan M, Meader A, Tomasek J, Howeard E, Ballard J: Cytosolic delivery and characterization of the TcdB glycosylating domain by using a heterologous fusion protein. Infection and Immunity 2001; 69(1)599-601.
43. Ballard J, Doling A, Beauregard K, Collier R, Starnbach M: Anthrax toxin-mediated delivery in vivo and in vitro of a cytotoxic T-lymphocyte epitope from ovalbumin. Infection and Immunity 1998 66(2)615-619.
44. von Moltke J, Trinidad N J, Moayeri M, Kintzer A F, Wang S B, van Rooijen N, Brown C R, Krantz B A, Leppla S H, Gronert K, Vance R E: Rapid induction of inflammatory lipid mediators by the inflammasome in vivo. Nature 2012; 490(7418)107-11.
45. Zeiser J, Gerhard R, Just I, Pich A: Substrate specificity of clostridial glucosylating toxins and their function on colonocytes analyzed by proteomics techniques. Journal of Proteomics Research 2013, 12(4)1604-1608.

### Example 2

### Cytotoxicity of the Vibrio vulnificus MARTX Toxin Effector DUF5 is Linked to the C2A Subdomain

Reference is made to Antic et al., Proteins. 2014 October; 82(10):2643-56, the content of which is incorporated herein by reference in its entirety.

Abstract

The multifunctional-autoprocessing repeats-in-toxin (MARTX) toxins are bacterial protein toxins that serve as delivery platforms for cytotoxic effector domains. The domain of unknown function in position 5 (DUF5) effector domain is present in at least six different species' MARTX toxins and as a hypothetical protein in Photorhabdus spp. Its presence in Vibrio vulnificus MARTX toxin increases potency of the toxin in mouse virulence studies, indicating DUF5 contributes to pathogenesis. In this work, DUF5 is shown to be cytotoxic when transiently expressed in HeLa cells. DUF5 localized to the plasma membrane dependent upon its C1 domain and the cells become rounded dependent upon its C2 domain. Both full-length DUF5 and the C2 domain caused growth inhibition when expressed in Saccharomyces cerevisiae. A structural model of DUF5 was generated based on the structure of Pasteurella multocida toxin facilitating localization of the cytotoxic activity to a 186 amino acid subdomain termed C2A. Within this subdomain, alanine scanning mutagenesis revealed aspartate-3721 and arginine-3841 as residues critical for cytotoxicity. These residues were also essential for HeLa cell intoxication when purified DUF5 fused to anthrax toxin lethal factor was delivered cytosolically. Thermal shift experiments indicated that these conserved residues are important to maintain protein structure, rather than for catalysis. The Aeromonas hydrophila MARTX toxin DUF5₁ₕ domain was also cytotoxic, while the weakly conserved C1-C2 domains from P. multocida toxin were not. Overall, this study is the first demonstration that DUF5 as found in MARTX toxins has cytotoxic activity that depends on conserved residues in the C2A subdomain.

Introduction

Multifunctional-autoprocessing repeats-in-toxins (MARTX) toxins are large protein toxins (3500-5300 aa) secreted by Gram-negative bacteria¹. These toxins carry from 1 to 5 protein effector domains, but also function as a delivery platform for transfer of these effector domains across the eukaryotic cell plasma membrane. These domains are then excised from the holotoxin by autoprocessing and released to the eukaryotic cell cytosol²⁻⁴ where they function as “effectors” freed from the translocation system of the toxin²⁻⁴. Among the various MARTX toxins of different mammalian, aquatic, and insect pathogens, a total of 10 different effector domains are carried by MARTX toxins, although the number and positional organization of the arrayed effectors vary across strains and species¹. The effector domain repertoire of the toxins can be exchanged by uptake of exogenous DNA and incorporation of the new sequences and/or loss of old sequences by homologous recombination resulting in novel toxins in different strains of the same species^{5,6}.

Within the target cell, the effector domains are thought to each have cytopathic or cytotoxic activity such that the overall role of the toxin in the eukaryotic cell is the sum of the activities of the effectors it delivers. Thus, it is important to individually characterize the function of each effector using genetics, biochemistry, and cell biology approaches to understand how an effector exchange will affect bacterial pathogenesis.

Among the 10 MARTX effector domains identified by sequence comparisons, only three have been functionally characterized¹. The actin crosslinking domain (ACD) covalently links actin monomers via an isopeptide bond leading to actin cytoskeletal destruction⁷⁻¹⁰. The Rho GTPase inactivation domain (RID) disables the Rho regulatory pathway resulting in loss of active Rho and thereby to cytoskeleton depolymerization^{11,12}. The ExoY domain is an adenylate cyclase¹³. The remaining seven MARTX toxin effector domains are uncharacterized but are often similar to domains of other large protein toxins¹.

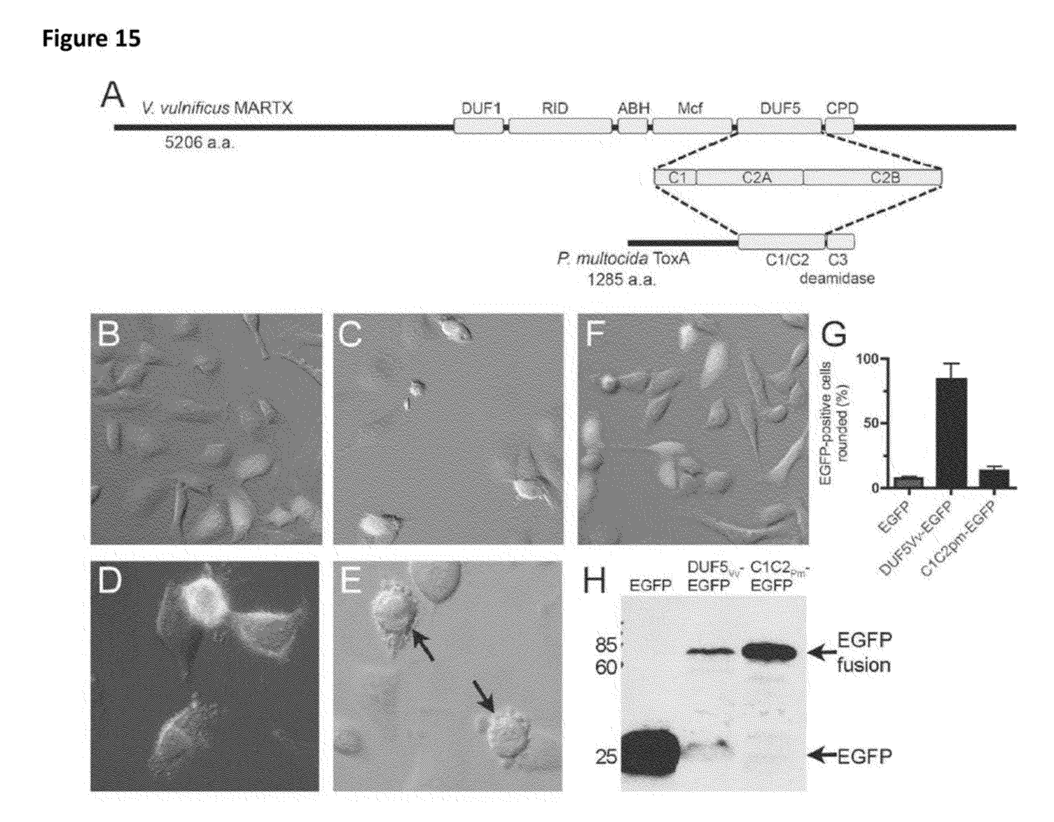

One of the domains of unknown function is known as DUF5, indicating its presence in the 5th effector domain position of the Vibrio vulnificus strain CMCP6 MARTX toxin where it was first recognized¹⁴ (holotoxin diagrammed in FIG. 15A). Within V. vulnificus, the presence of DUF5_{Vv} increases the potency of the toxin during mouse infection resulting in a lower LD₅₀ compared to an isogenic strain from which the effector domain was deleted or a naturally occurring strain that lost DUF5_{Vv} via a homologous recombination event5. Thus, DUF5_{Vv} is a virulence factor that increases the pathogenicity of the strains that carry it as a domain within the MARTX toxin.

DUF5_{Vv} was initially recognized to have sequence similarity to Pasteurella multocida toxin (PMT), whose carboxyl-terminus is composed of three domains: C1, C2, and C315. The C1Pm subdomain from PMT is known to be a four helical bundled membrane localization domain (4HBM)¹⁶. The conserved C1_{Vv} subdomain from DUF5_{Vv} has also been demonstrated to localize to the eukaryotic plasma cell membrane, where it binds anionic lipids via a basic-hydrophobicmotif^{12,17}. Structural determination by nuclear magnetic resonance of the isolated C1Pm and C1_{Vv} subdomains confirm both of these domains form a four helical bundle in solution^{18,19}.

However, none of the extensive characterization of PMT has revealed the function of its C2 domain. The PMT C3 domain is a deamidase enzyme with a catalytic cysteine residue that acts on the Gα subunits of trimeric G proteins²⁰⁻²³. It is notable that the sequence similarity of DUF5_{Vv} with PMT is limited to the C1 and C2 domains and DUF5_{Vv} does not share the C3 deamidase domain and thus DUF5_{Vv} is not expected to have a similar activity (FIG. 15A). DUF5 is present also within MARTX toxins of Aeromonas hydrophila, Yersinia kristensenii, Vibrio splendidus, and Xenorhabdus nemotophila1 and as the stand-alone hypothetical protein plu2400 in Photorhabdus sp.,²⁴ where it might be an effector with a distinct delivery mechanism such as Type III secretion or the Tc complex²⁵.

In this study, we initiated a de novo investigation on this protein of unknown function. We generated a structural model of DUF5_{Vv} based on the structure of the PMT C-terminus¹⁵. We then show that ectopic expression of the domain in HeLa cells is cytotoxic. In Saccharomyces cerevisiae, expression of the DUF5_{Vv} causes growth inhibition. The toxic effect in HeLa cells is mapped to a 186 amino acid C2A subdomain and shown to require an Asp and Arg residue. Overall, these studies mark our initial efforts to establish that DUF5 is a bona fide MARTX toxin effector.

Materials and Methods

Cell Lines, Media, Reagents and Plasmids

HeLa epithelial cells were grown at 37° C. with 5% CO₂ in Dulbecco's Modified Eagle Medium (DMEM, Life Technologies Gibco) with 10% fetal calf serum (Gemini Bio-Products, West Sacramento, Calif.), 100 U/ml penicillin, and 1 μg/ml streptomycin. J774 macrophages, COS7 fibroblasts, and HEp-2 epithelial cells were grown in identical conditions. E. coli DH5α, TOP10 (Life Technologies Invitrogen) and BL21(DE3) were grown at 37° C. in Luria-Bertani (LB) liquid or agar medium containing either 100 μg/ml ampicillin or 50 μg/ml kanamycin as needed. S. cerevisiae strain InvSc1 (Invitrogen) was grown on YPD liquid or agar medium at 30° C. or commercial synthetic complete (SC-ura) supplemented with yeast nitrogen base (MP Biomedicals) as detailed below. Media components and common reagents were obtained from Sigma-Aldrich, Fisher, or VWR and common restriction enzymes and polymerases from New England Biolabs or Invitrogen. Custom DNA oligonucleotides and gBlocks were purchased from Integrated DNA Technologies (Coralville, Iowa). Plasmids were prepared either by alkaline lysis with precipitation in ethanol or purified using Epoch spin columns according to manufacturer's recommended protocol.

Alignments and Structural Modeling

Proteins with homology to DUF5_{Vv} from strain CMCP6 were identified using BLASTP26 at the National Center for Biotechnology Information website. Amino acid sequences were trimmed to DUF5_{Vv} homology region and aligned with CLUSTALW using MacVector 12.6.0. The DUF5_{Vv} and DUF5_{Vv} D3721A protein sequences were also aligned to the pdb database using HHpred27 and a pdb structural model built based on published PMT structure (pdb 2EBF15) using Modeller 28. Figures were generated from the structural model using MacPyMol.

Construction of Plasmids for Ectopic Expression in HeLa Cells and Yeast

DNA corresponding to coding sequence for amino acids 3579-4089 of the V. vulnificus rtxA1 gene (GI: 27366913; _{VV}2_{—}0479) was amplified from purified V. vulnificus CMCP6 chromosomal DNA using Pfx50 DNA polymerase (Invitrogen) and primers 1 and 2 (5′-gagctagcatgggtgataaaaccaaggtcgtggattta (SEQ ID NO:55), and 5-gccgtcgaccaaactgcccttgaacgtgatcttcggttt (SEQ ID NO:56)). The insert was digested with enzymes NheI and SalI and ligated into the similarly digested vector. For expression of the P. multocida toxA gene sequence corresponding to aa 573-1113 of PMT from strain 4533 (GI:149228008), a synthetic codon optimized double stranded DNA sequence was obtained from GenScript (Piscataway, N.J.) in pUC57 and subcloned into pEGFP-N3 via the HindIII and BamHI sites.

DNA sequences above were similarly amplified except with novel EcoRI and KpnI restriction sites incorporated into the oligonucleotides for transfer into yeast expression vector pYC2 NT/A (Invitrogen) using primers 6, 7, 8 and 11 (5′-aaggtaccgtttatcggtaagatgcaagttgcc (SEQ ID NO:30), 5′-agaattctcacaaactgcccttgaacgtgatc (SEQ ID NO:31), 5′-aaggtaccgggtgataaaaccaaggtcgtg (SEQ ID NO:32), and 5′-aaggtaccggatattgacgcttgggatcgt, SEQ ID NO:33).

Ectopic Expression of EGFP Fusion Proteins in HeLa Cells

Plasmids for transfection were prepared using the Qiagen Midi Prep kit. HeLa cells grown to approximately 80% confluency were transfected using Fugene HD (Promega) and 2 μg plasmid DNA at a 4:1 ratio for 5 h after which fresh media was exchanged. For western blotting, cells were collected 24 h after transfection in 2×SDS-PAGE buffer, boiled for 5 min, and the proteins were separated by SDS-PAGE and transferred to nitrocellulose by the tank blot method. Western blotting was done as previously described17 using anti-GFP antibody conjugated to horseradish peroxidase (Milteny Biotec 130-091-833) at a 1:1000 dilution. SuperSignal West Pico Chemiluminescent Substrate and autoradiography was used for detection.

For microscopy, HeLa cells were grown in 35 mm MatTek glass bottom microwell dishes to approximately 60% confluency and transfected as described above. Live cells were imaged 24 h after transfection by epifluorescence and differential interference contrast (DIC) microscopy at 200× using the Andor Spinning Disk confocal microscope. Images were overlayed using NIH ImageJ64 and assembled into figures using Adobe Photoshop CS6. Rounded cells were manually counted from at least 3 different transfections. Histograms of representative cells were plotted using GraphPad Prism 4.0 or 6.0.

Purification of Proteins Fused to Anthrax Toxin Lethal Factor N-Terminus

Plasmid vector pRT24 is a variant of pABII29 in which the coding sequence for amino acids 1-254 of anthrax toxin lethal factor (LFN) are expressed with an N-terminal His-tag under control of the T7 promoter. The plasmid was modified to replace the single BamH1 cloning site with an oligonucleotide that introduces the TEV cleavage site and ligation independent cloning site from pMCSG730. DUF5_{Vv} DNA sequences were amplified with primers 3 and 4 (5′-tacttccaatccaatgctgataaaaccaaggtcgtggtcgattta and 5′-ttatccaatgtgaaagagcggtatttgcgccactcaa) and integrated into pRT24 by ligation-independent cloning³⁰. A stop codon after the codon for Thr3765 was introduced to generate a sequence that would be truncated after C2A. Site-directed mutagenesis was then used to alter codons D3721 and R3841 to Ala as described above. DUF5 from A. hydrophila fused to LFN (LFN-DUF5_{Ah}) was generated in the same manner as LFNDUF5_{Vv} except using primers 14 and 15 (5′-tacttccaatccaatgctccgggcaaaacggtggtgacg, and 5′-ttatccacttccaatgctagacatcggcgtactcgacccgc) to amplify the sequence corresponding to the MARTX toxin aa 3069-3570 (GI: 117618727) from chromosomal DNA prepared from A. hydrophila 7966 obtained from the American Type Culture Collection.

LFN and LFN fusion proteins were expressed in E. coli BL21 (DE3). Briefly, overnight cultures were diluted 1:100 in fresh LB containing the 100 μg/ml ampicillin and grown to OD600=1.0 at 37° C. before inducing the cultures with 1 mM isopropyl β-D-1-thiogalactopyranoside (IPTG) for 4 h at 32° C., except for LFNDUF5_{Vv} expression which was induced with 0.5 mM IPTG and cells were grown at 30° C. E. coli cells were harvested by centrifugation at 10,100×g and resuspended in 100 ml Urea Buffer A (500 mM NaCl, 20 mM Tris pH 7.4, 5 mM imidazole and 8 M urea with addition of protease inhibitor tablets (cOmplete, EDTA-free purchased Roche Applied Sciences). Resuspended cells were sonicated using a Branson Sonifier model 102 C for 7 min at 50% amplitude with the standard disruptor. Crude lysates were centrifuged at 17,600×g for 30 min to remove particulates and the remainder of the lysate was filtered across a PALL Acrodisc 0.45μ syringe filter. Lysate was loaded onto a 1 ml GE Healthcare HisTrap column using the AKTA purifier protein purification system (GE Healthcare). Column was washed with 5 ml Urea Buffer A with 10 mM imidazole, followed by 5 ml 50 mM imidazole buffer to remove contaminating proteins. His-tagged LFN proteins were eluted using an imidazole gradient from 50 to 250 mM. Peak fractions corresponding to the protein of interest were collected, pooled, and dialyzed to remove imidazole into a buffer containing 500 mM NaCl, 20 mM Tris, and 2 M urea, pH 7.4. Proteins were further purified by gel exclusion chromatography in the same buffer using a 16×100 Superdex 200 column (GE Healthcare). Purified proteins were concentrated using Millipore Amicon Ultra 30K spin concentrators and glycerol was added so that the final buffer was 300 mM NaCl 12 mM Tris pH 7.4, 1.2M urea, 20% glycerol. Protein concentration was determined using the NanoDrop ND1000, and purity was estimated using SDS-PAGE. Proteins were stored at −80° C. until used.

Protective antigen (PA) was purified from the soluble fraction of E. coli BL21(DE3). Cells were grown at 37° C. to OD600=0.8, then the culture was induced with 1 mM IPTG for 4 h at 30° C. Bacterial culture was harvested by centrifugation, then resuspended in 500 mM NaCl, 20 mM Tris, 5 mM imidazole, pH 8.0. Lysate was prepared as for LFN fusion proteins above except buffers did not contain urea. Sizing was performed as described above in 500 mM NaCl, 20 mM Tris pH 8.0 buffer.

Intoxication of Mammalian Cells with LFN Fusion Proteins and PA

All cell types were grown in 24 well tissue culture treated dishes (Falcon). 7 nM PA and 3 nM LFN-fusion proteins were added to 1 ml culture media overlaying the cells. Cells were incubated for 24 or 48 h at 37° C. in 5% CO2, after which cells were imaged at 100× by phase microscopy using a Nikon CoolPix 995 digital camera affixed to a Nikon TS Eclipse 100 microscope. For quantification, rounded cells were manually counted representing at least 3 independent experiments and results were graphed as histograms using GraphPad Prism 4.0 or 6.0.

Assay for Cell Lysis

Lactate dehydrogenase (LDH) release from intoxicated cells was determined using the Cytotox 96 Non-Radioactive Cytoxicity Assay (Promega). After intoxication, 50 μl of culture media was removed from each well, mixed with 50 μl of reaction reagent, and incubated at room temperature protected from light for 30 min. Upon addition of stop solution, absorbance was measured at 490 nm. For determination of total LDH, cells from the same wells were lysed by addition of Triton X-100 to the residual media to a final concentration of 0.1% and then sampled and assayed as described above to determine the maximum lysis value for each well. Percent cell lysis was calculated using the formula

$$( \frac{A 490 media}{( A 490 media + cells )} ) exp - ( \frac{A 490 media}{( A 490 media + cells )} ) untreated * 100.$$

Assessment of Yeast Growth Inhibition

S. cerevisiae strain InvSc1 was grown in YPD broth prior to transformation. Yeast cells were transformed using a PLATE solution method and transformants selected using SC agar medium without uracil, supplemented with glucose as previously described³¹. Transformed yeast cells were inoculated into liquid glucose synthetic complete medium (without uracil) and grown overnight at 30° C. The next day, cultures were centrifuged and washed three times with sterile water. Each sample was resuspended in water and OD₆₀₀ was measured for each using Beckman Coulter DU530 Spectrophotometer. All samples were normalized to OD₆₀₀=0.5 and then were 10-fold serially diluted. 5 μl of each dilution was spotted on solid agar selective medium (-uracil) with either 20 mg/ml glucose or 20 mg/ml galactose and 10 mg/ml raffinose. The plates were incubated at 30° C. for 3 days before growth was assessed and plates photographed using a digital camera. For growth cures, OD₆₀₀ of overnight cultures was measured and inoculi were normalized to each other and then diluted into 50 ml of SC medium containing 20 mg/ml galactose and 10 mg/ml raffinose (-uracil) to induce expression from the plasmid. OD₆₀₀ was measured every 2 h for 12 h to document growth patterns.

Alanine Scanning Mutagenesis

Site-directed mutagenesis to introduce an alanine or stop codon at locations noted in text was carried out using PfuTurbo DNA polymerase (Invitrogen) and custom oligonucleotides designed via Agilent PrimerDesign software. After amplification, DNA was treated with DpnI and transformed to E. coli TOP10. Isolated plasmids were sequenced to confirm gain of the desired mutation and to check for absence of unintended mutations during DNA amplification. Double mutant D3721R/R3841D in pYC-DUF5 plasmid was generated by cohesive end cloning of a synthetic DNA gBlock containing the R3841D mutation in exchange for the wild type sequence via flanking BamHI and AatII restriction enzyme sites (5′-atctttatggtcgcgattgaagaagccaacggtaaacacgtaggtttgacggacatgatggttcgttgggccaatgaagaac catacttggcaccgaagcatggttacaaaggcgaaacgccaagtgaccttggttttgatgcgaagtaccacgtagatctagg tgagc, SEQ ID NO:34). Purification of recombinant 6×HIS-tagged proteins for fluorescence thermal shift assays DNA corresponding to DUF5_{Vv} was inserted into the overexpression vector pMSCG7 by ligation independent cloning using primers 12 and 13, (5′-tacttccaatccaatgctcaagagctgaaagaaagagcaaaag, SEQ ID NO:35 and 5′-tacttccaatccaatgctcaagagctgaaagaaagagcaaaag, SEQ ID NO:36). Additional mutations were generated by site directed mutagenesis. Plasmids were transformed into E. coli BL21 (DE3) for purification. Cells were grown to OD₆₀₀=0.8 at 37° C. The temperature was reduced to 18° C. and protein expression induced by the addition of IPTG to a final concentration of 1 mM. Cells were grown overnight with shaking and then harvested by centrifugation. Bacteria were resuspended in a buffer containing 50 mM Tris (pH 8.3), 500 mM NaCl, 0.1% Triton X-100, and 5 mM β-mercaptoethanol and lysed by sonication. After centrifugation at 30,000×g for 30 min, the soluble lysate was filtered through a 0.22 μm membrane and loaded onto a 1 ml HisTrap column using the ÄKTA purifier protein purification system (GE Healthcare). After washes with 50 mM Tris, 500 mM NaCl, 50 mM Imidazole pH 8.3, the proteins were eluted in the same buffer with 500 mM imidazole. Proteins were further purified by gel filtration chromatography (Superdex 75 (16/60), GE Healthcare) in buffer containing 10 mM Tris-HCl, 500 mM NaCl, 5 mM β-mercaptoethanol, pH 8.3.

Fluorescence Thermal Shift Assay

The experiment was performed using a 96-well thin-wall PCR plate (Axigen). 20 μl reactions consisted of 2 μM protein in a solution of 5×SYPRO orange dye (Life Technologies), 0.1 mM HEPES, 150 mM NaCl, pH 7.5. Fluorescence intensity was monitored using the StepOnePlus™ Real-Time PCR Systems (Life Technologies) instrument. Samples were heated from 25° C. to 95° C. at a scan rate of 1° C./min. Tm values were extrapolated using Protein Thermal Shift™ Assay software (Life Technologies).

Results

DUF5_{Vv}, but not C1C2Pm, is Cytotoxic when Ectopically Expressed in HeLa Cells

To determine if DUF5 is a bona fide effector with cytotoxic effects on cells, the DNA sequence corresponding to V. vulnificus aa 3579-4089 (DUF5_{Vv}) was amplified and cloned into ectopic expression vector pEGFP-N3 for expression of DUF5_{Vv} as a fusion to EGFP under control of the CMV promoter. The plasmid was transformed into cultured HeLa cervical carcinoma epithelial cells and EGFP-positive cells were imaged after 24 hr. Cells expressing EGFP had a normal, cuboidal shape with less than 8% of cells rounded (FIG. 15B). By contrast, 82% of cells ectopically expressing the DUF5_{Vv}-EGFP fusion were small and rounded and many of the cells showed signs of blebbing indicating necrosis (FIG. 15B,D). Some cells that had not yet fully rounded or necrosed showed DUF5_{Vv}-EGFP localized to the cell periphery, consistent with the presence of the C1 plasma membrane localization domain (FIG. 15C). Western blot detection of the DUF5_{Vv}-EGFP fusion showed less total protein than detected for the EGFP-expressing control cells (FIG. 15H), indicating that expression of this fusion protein was toxic to cells and many cells expressing the DUF5_{Vv}-EGFP may have detached.

DUF5_{Vv} has 24% sequence identity with the C1-C2 domains of PMT (C1C2Pm) (FIG. 15A). Since the toxA gene is carried on a bacteriophage with a low GC content (35% GC), a eukaryotic codon-optimized, synthetic copy of toxA sequences corresponding to C1C2Pm was obtained and expressed in cells generating a protein similar in size to DUF5_{Vv}-EGFP (FIG. 15H). Cells expressing C1C2Pm-EGFP appeared similar to EGFP-control expressing cells (FIG. 15F). These results support previous data^{20,21,32,33} that all toxic activities of PMT are due to the C3 deamidase domain that is absent in DUF5_{Vv}. Further, these data show that the cytotoxic activity of DUF5_{Vv} may not be conserved in C1C2Pm, at least in HeLa cells.

Cytotoxicity of DUF5_{Vv} in HeLa Cells is Linked to the C2A Domain

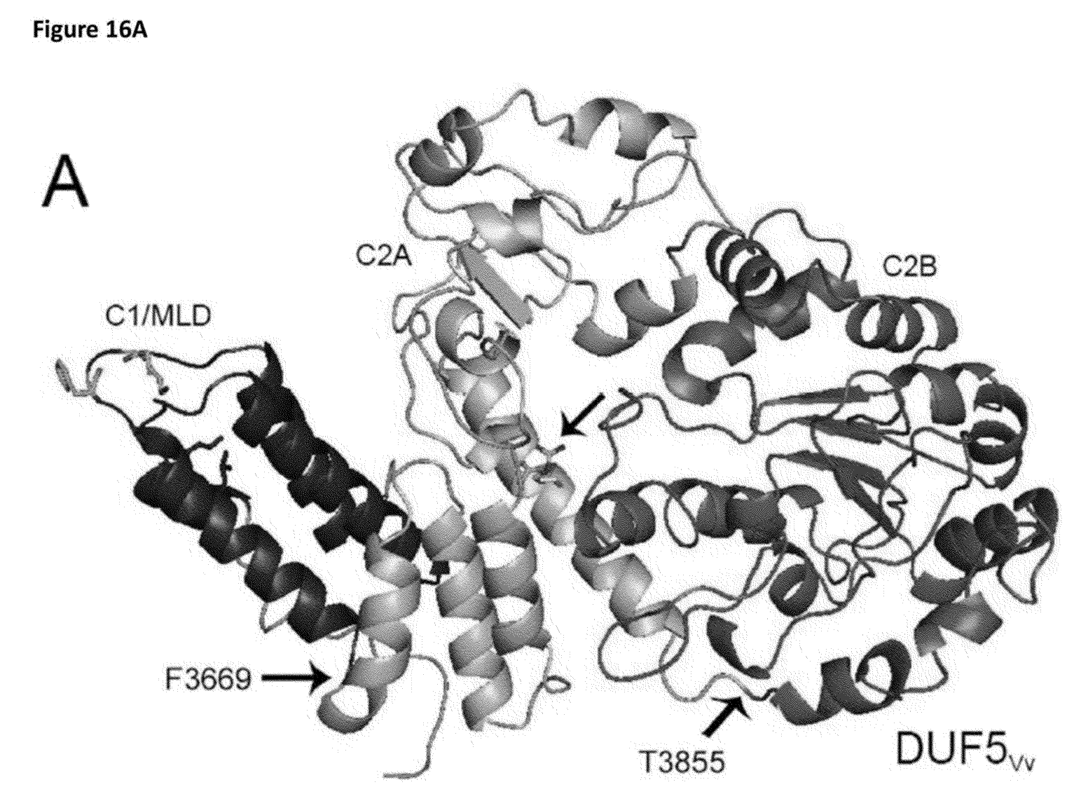

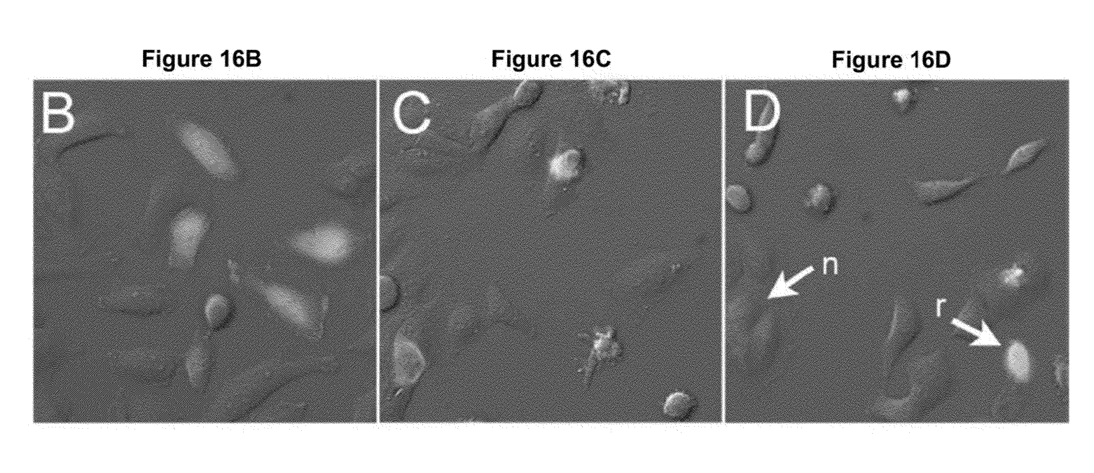

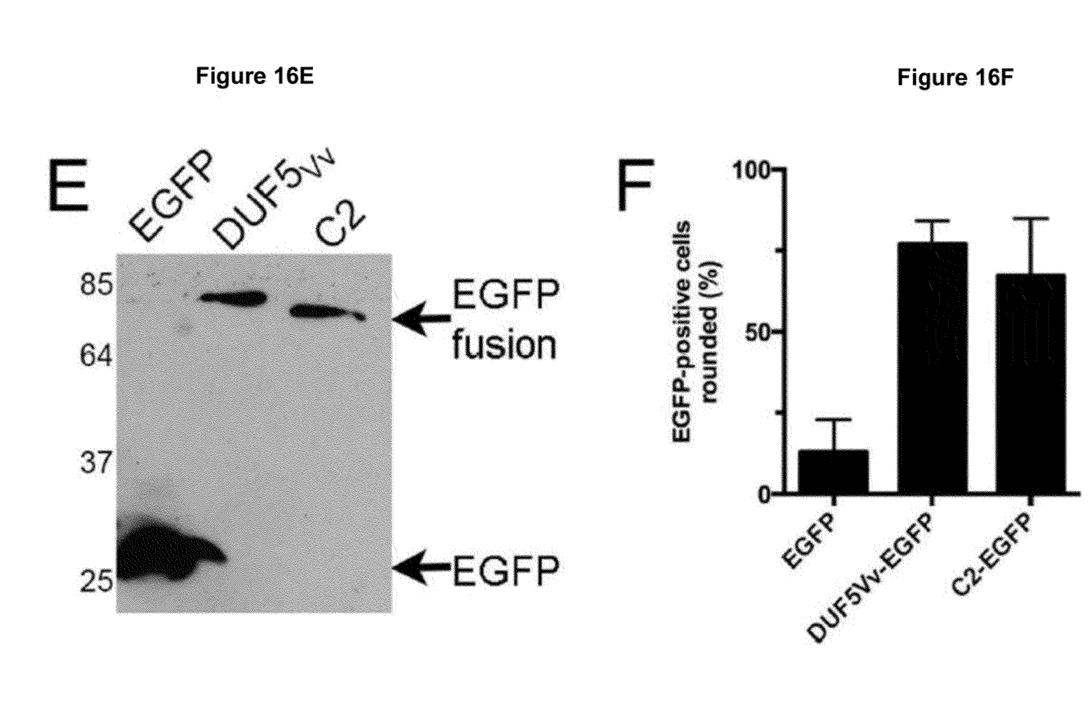

Despite the absence of functional conservation, C1C2Pm and DUF5_{Vv} may share structural conservation, although the function of the domains diverged. A structural model of DUF5_{Vv} was generated based on the PMT structures¹⁵. Based on this model, the amino acids of DUF5_{Vv} responding to the C1_{Vv} and C2_{Vv} domain were identified. Upon deletion of gene sequences for the C1_{Vv} subdomain, the C2_{Vv}-EGFP fusion is no longer localized to the cell periphery. Those cells highly expressing C2_{Vv}-EGFP appear rounded, while low expressing cells remained normal (FIG. 16D). These data are consistent with C2_{Vv} being required for cytotoxicity and C1_{Vv} being required for efficient delivery to the plasma membrane.

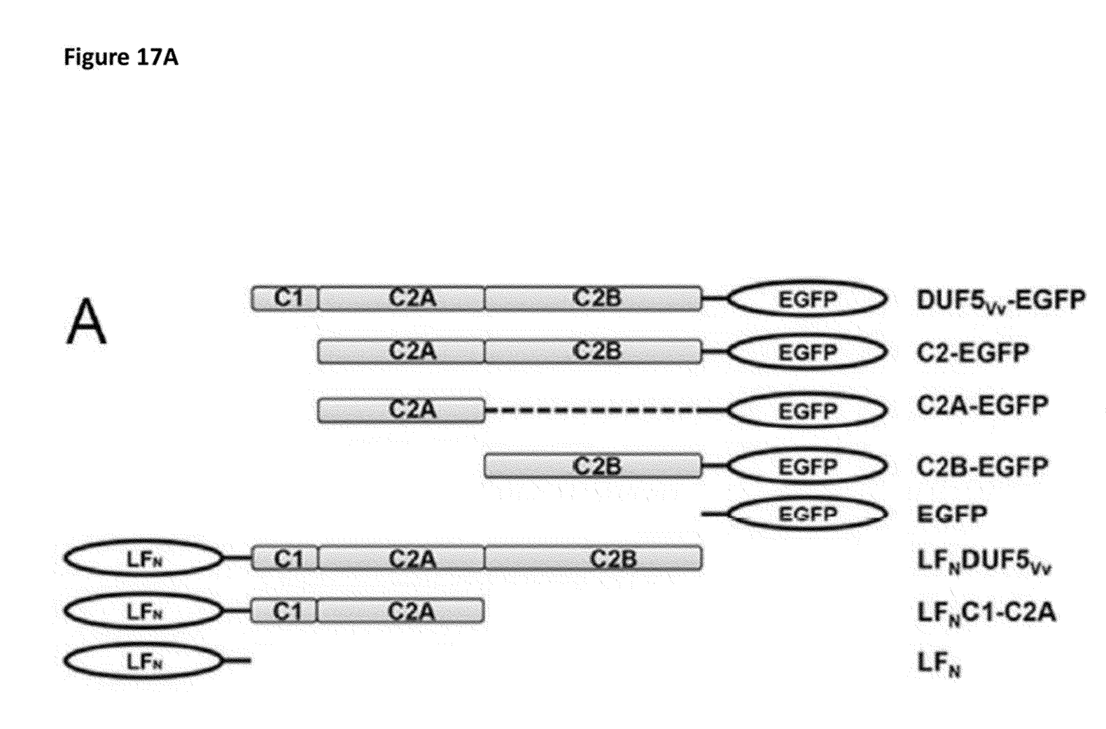

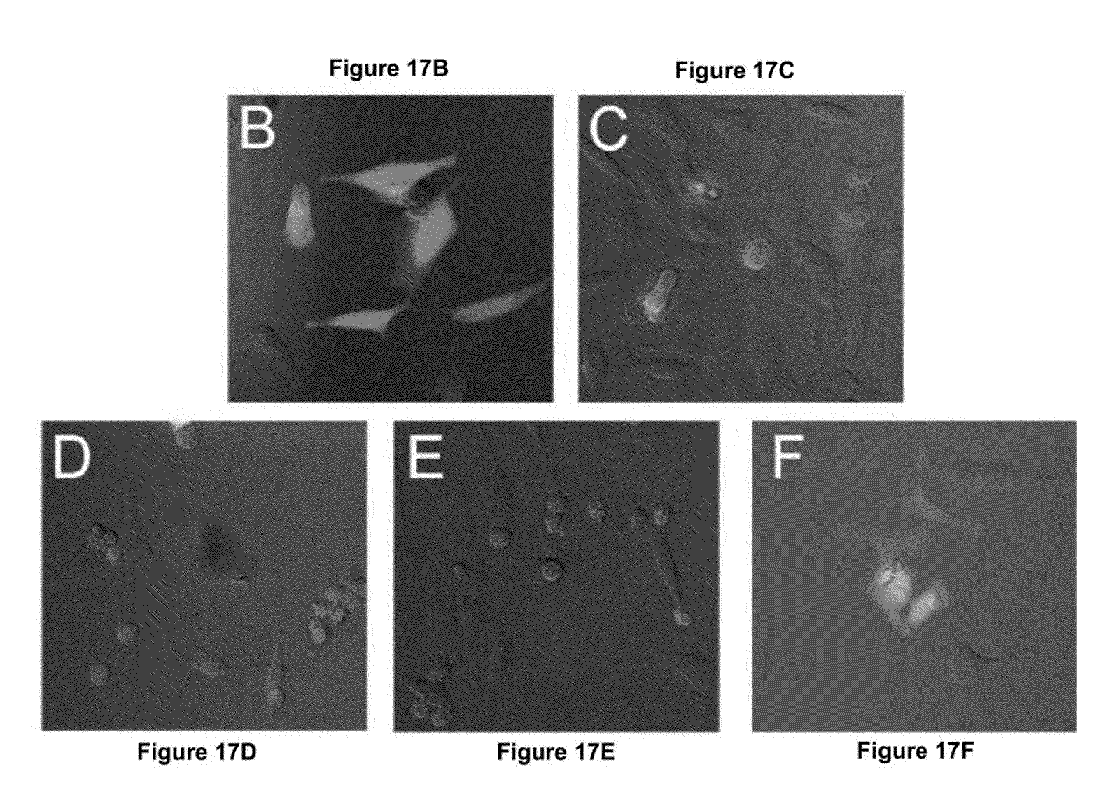

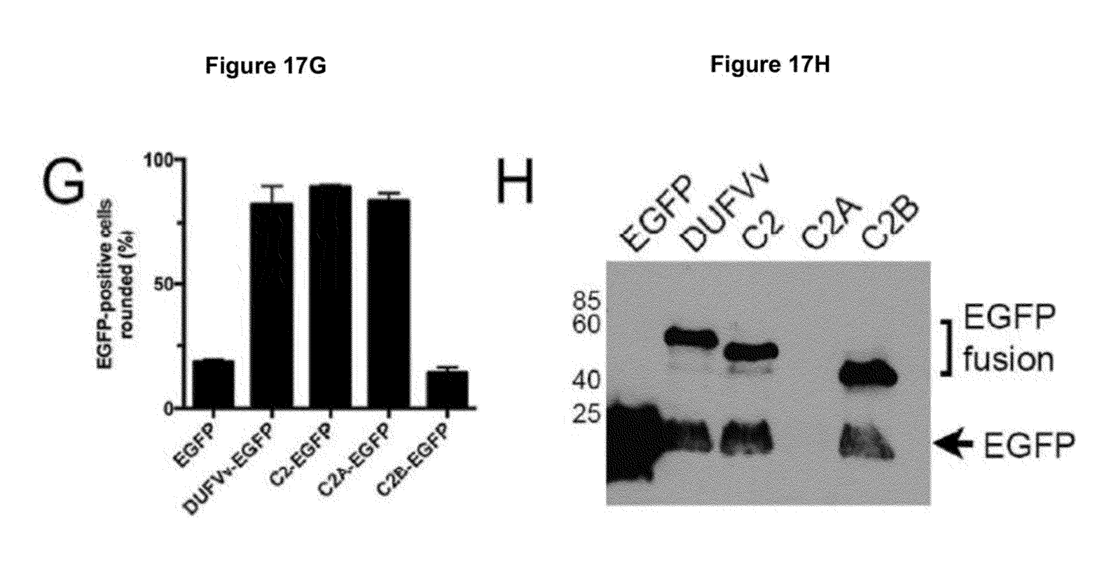

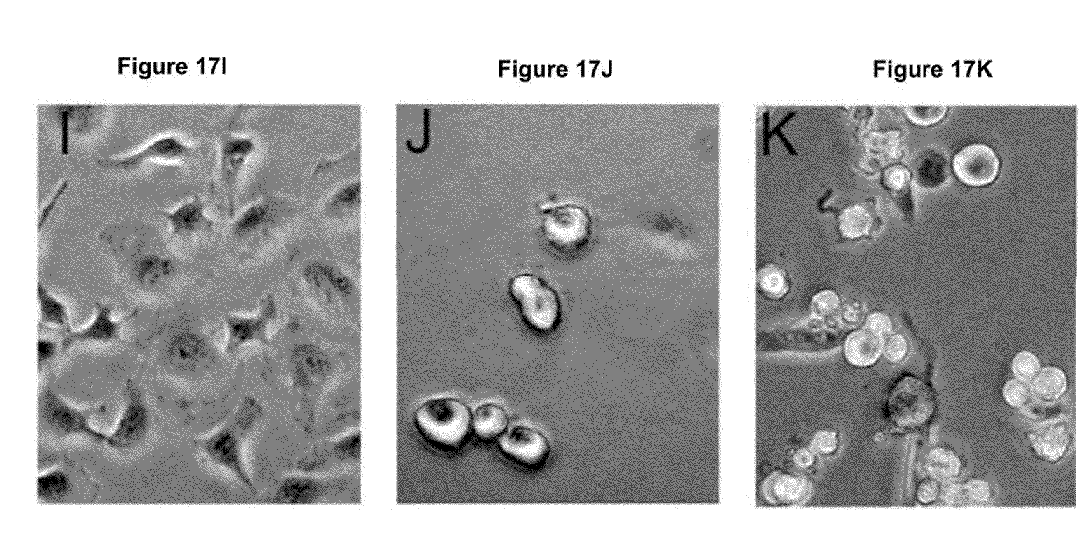

In addition, as shown also by two recent bioinformatics studies^{34,35}, the structural model showed that C2_{Vv} could be split into two subdomains, C2A_{Vv} and C2B_{Vv} (FIG. 16A). To determine if the cytotoxic activity of C2_{Vv} is linked its C2A or C2B subdomain, DNA corresponding to the individual subdomains was cloned fused to egfp and expressed in HeLa cells. Cells ectopically expressing only C2A_{Vv}-EGFP were highly necrotic, while cells expressing C2B alone appeared normal (FIG. 17B-G) and produced EGFP-fusion protein detectable by western blotting (FIG. 17H). However, due to the severe toxicity of C2A alone resulting in poor sample recovery, a corresponding fusion protein could not be detected by western blotting to confirm expression (FIG. 17H).

As an alternative verification of the cytotoxicity associated with C2A_{Vv}, both full-length DUF5_{Vv} and C1-C2A from V. vulnificus were purified fused to His-tagged B. anthracis LFN that is often used as a bioporter for toxin effectors in the absence of the holotoxin^{7,11,29,36}. The purified proteins were insoluble in less than 2M urea, but nevertheless retained toxicity after delivery to cells by PA. The snap dilution out of urea in the tissue culture media likely allowed folding of the LFN domain and the protein then associated with PA for translocation and successful refolding of the DUF5_{Vv} domain within the cytosol. Notably, both the full-length protein (FIG. 17J) and the C1-C2A fragment (FIG. 17K) resulted in rounding of cells confirming transfection studies that C2A is sufficient for cytotoxicity of DUF5_{Vv} in HeLa cells. Furthermore, LFNDUF5_{Vv} was cytotoxic to other mammalian cell types as well, including J774 macrophages, COST fibroblasts, and HEp-2 epithelial cells (Table 1).

TABLE 1
Cell lines susceptible to DUF5_{Vv} cytotoxicity^{a}

| | Rounding induced | |
|---|---|---|
| | by LF_{N}DUF5_{Vv}? | |
| Cell Line | +PA | −PA |
| HeLa human cervical carcinoma | + | − |
| COS7 African green monkey fibroblast | + | − |
| J774 murine macrophage | + | − |
| Hep-2 human laryngeal epithelial | + | − |

^{a}Cells were intoxicated with LF_{N}DUF5_{Vv} in the presence (+) or absence (−) of PA. After 24 hr, rounding was observed by phase microscopy. Intoxication conditions were the same as reported in FIG. 17.

DUF5_{Ah} from A. hydrophila is Also Cytotoxic

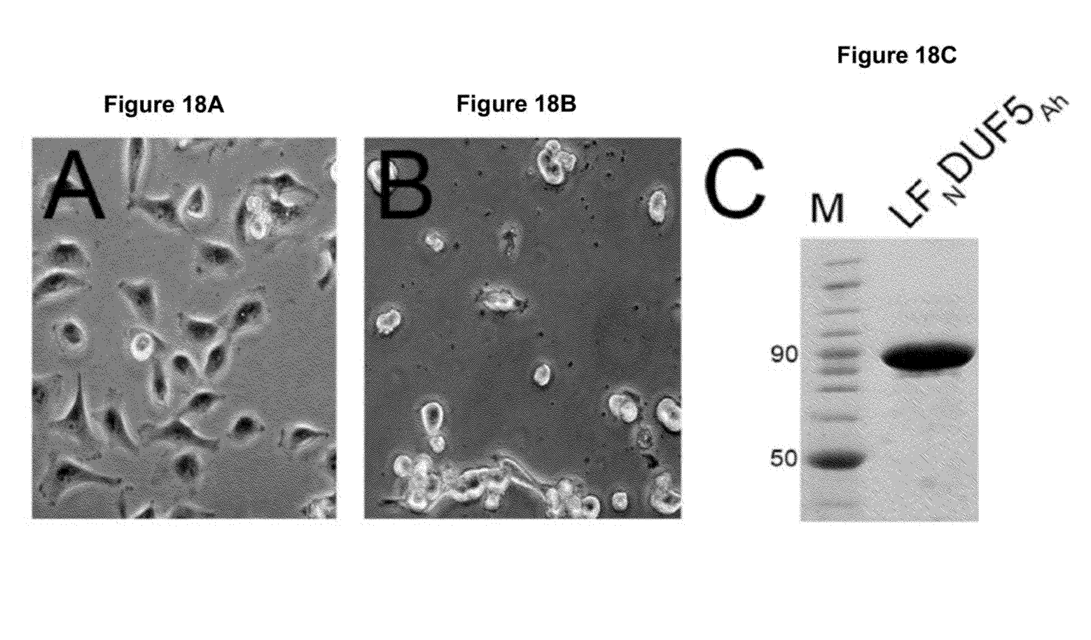

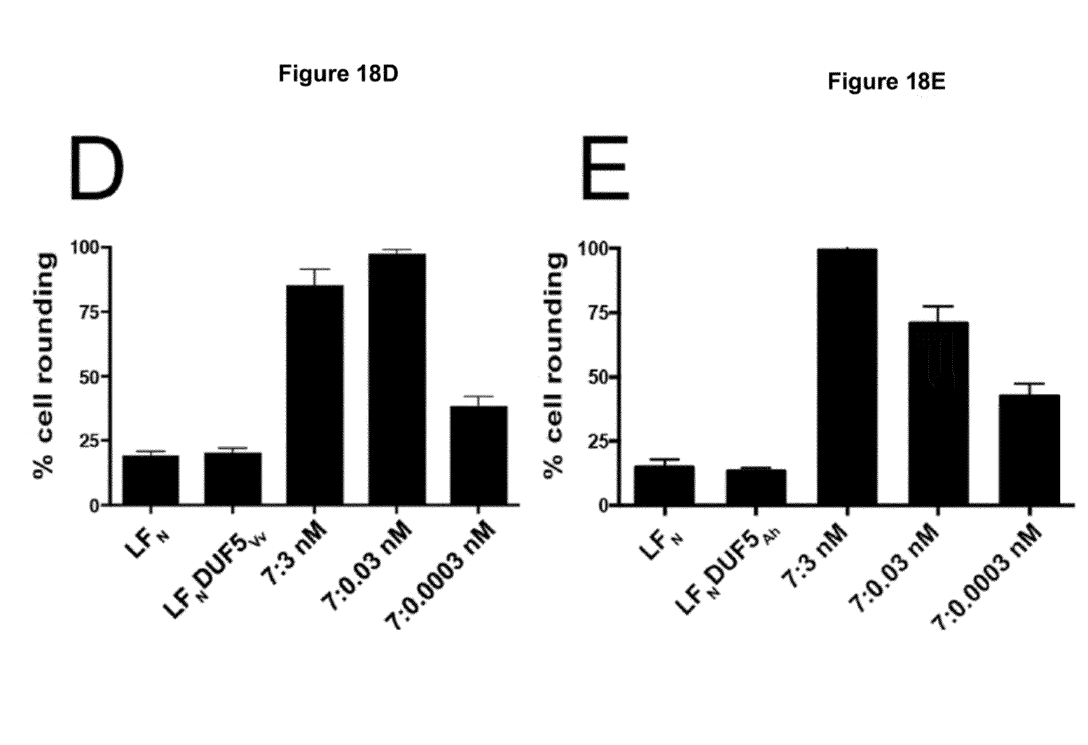

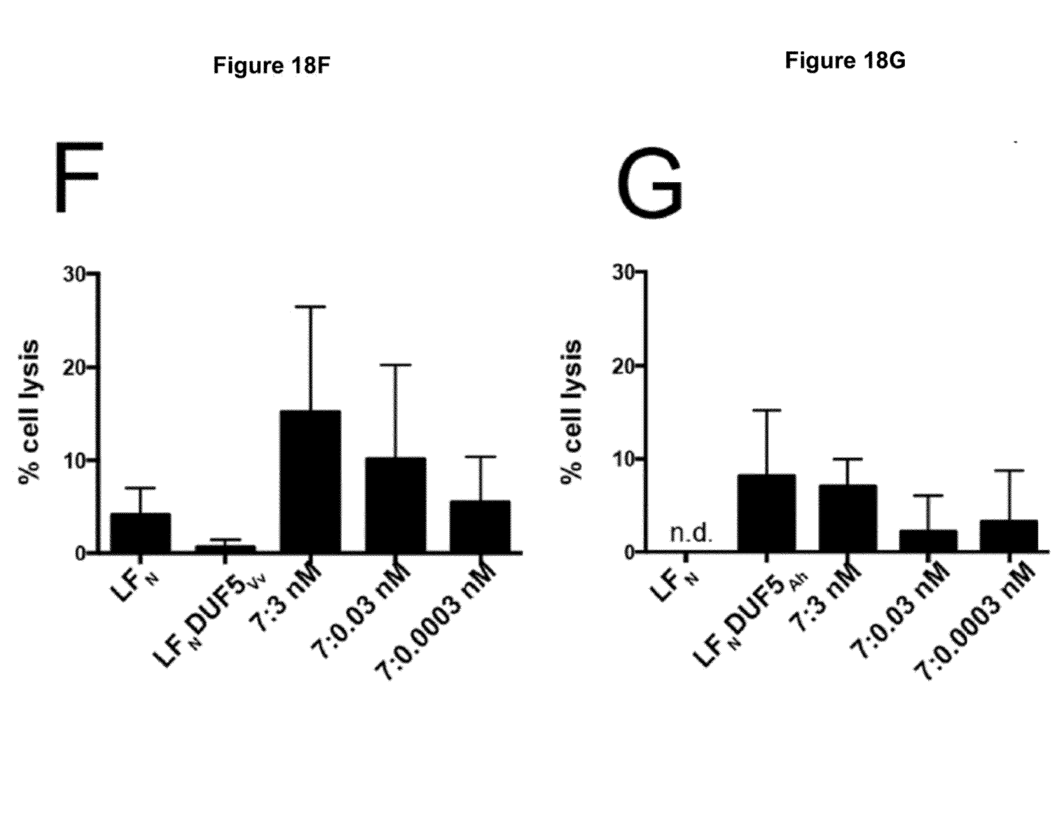

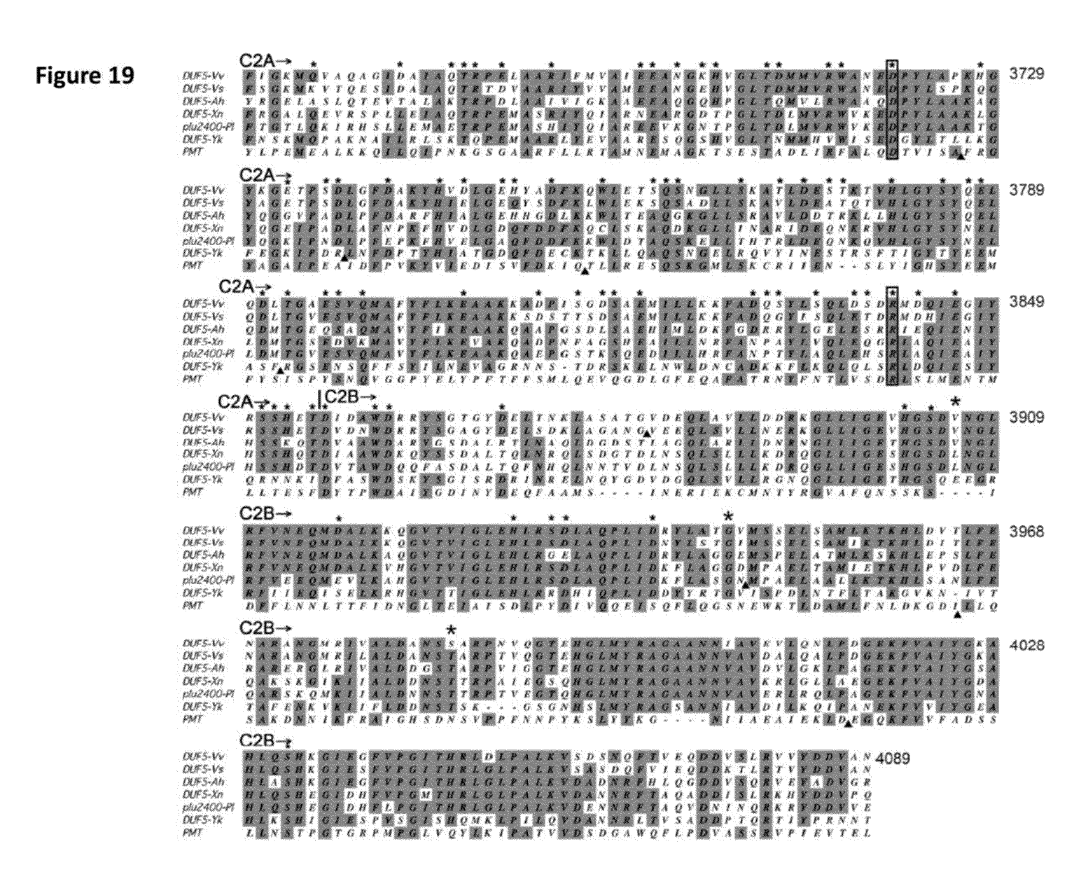

As shown in FIG. 19, proteins similar to DUF5_{Vv} and PMT C1-C2 subdomains are found as uncharacterized proteins from other bacterial species. To further explore the possibility that these proteins comprise a novel functional family of cytotoxins, the DUF5-like effector domain from the A. hydrophila MARTX toxin was cloned in fusion with LFN and delivered to HeLa cells via PA. Protein purity was assessed by SDS-PAGE in panel 4C. After intoxication it was observed that HeLa cells were rounded similarly to what is seen with LFN-DUF5 (FIG. 18 A,B) indicating that this effector domain also has cytotoxic function. Furthermore, the rounding efficiency is similar between the two toxins (FIG. 18 D, E) Finally, neither toxin induced cell lysis when delivered to HeLa cells, at any of the concentrations tested (FIG. 18 F, G).

Both Full-Length DUF5_{Vv} and C2 Alone Cause Growth Inhibition when Expressed in Yeast

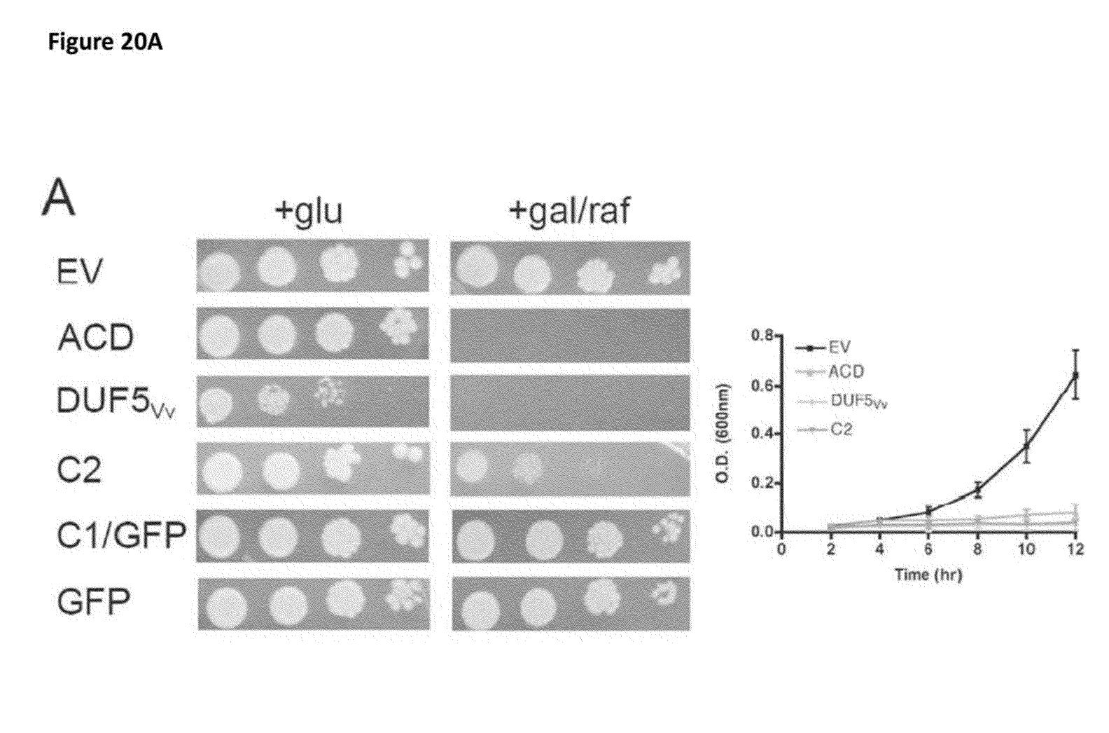

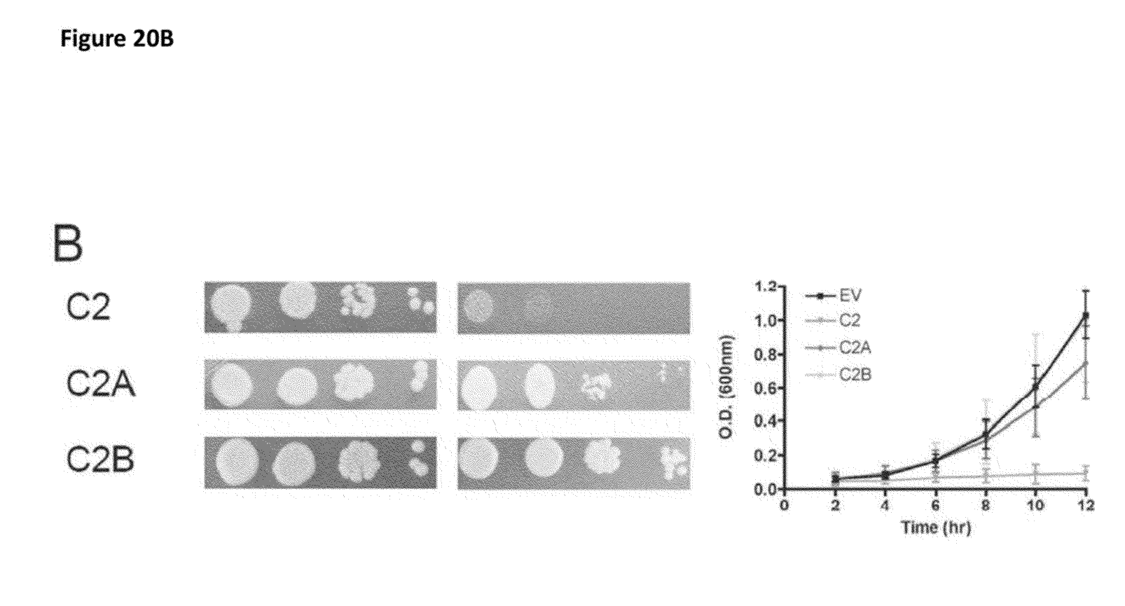

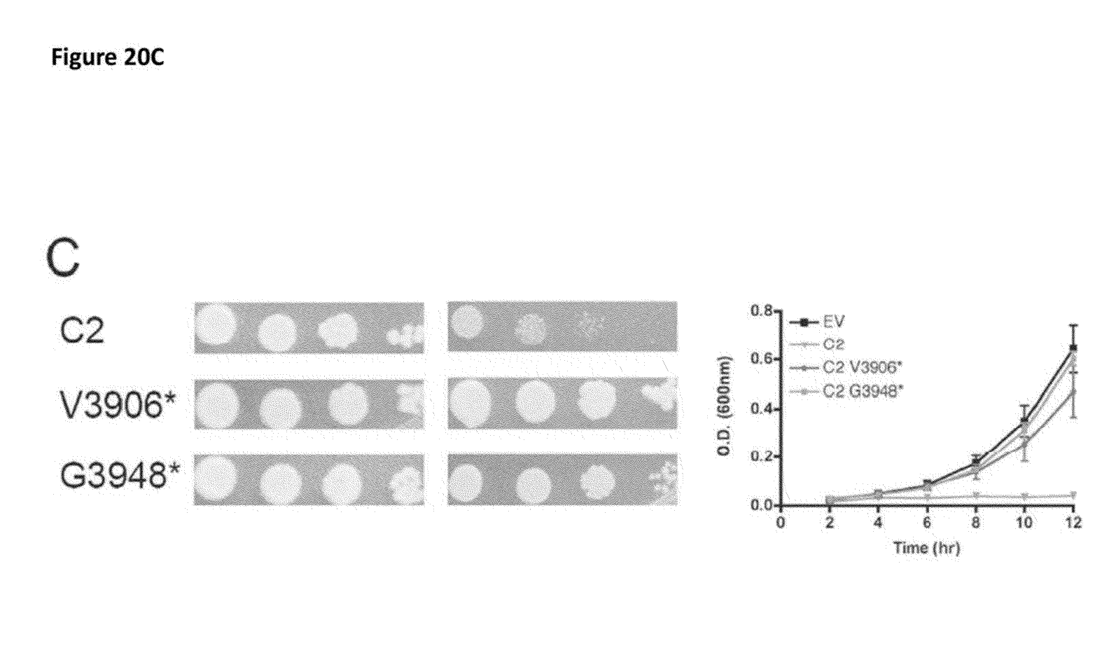

To further explore the function of DUF5_{Vv}, we tested if it would be toxic if expressed in S. cerevisiae. The gene sequence for DUF5_{Vv} was cloned into the yeast expression vector pYC2/NTA placing the gene under the control of a galactose-inducible promoter. When transformed into yeast, the DUF5_{Vv}-expressing yeast strain grew poorly under non-inducing conditions and showed no growth under inducing conditions on either plates or broth culture (FIG. 20A). Indeed, expression of DUF5_{Vv} was more toxic than the MARTX ACD effector domain that has been previously studied in yeast (FIG. 20A)³¹. Toxicity was reduced by removal of the C1 MLD such that cells expressing C2 alone were viable under non-inducing conditions with 100- to 1000-fold reduced plating efficiency on galactose and no growth in broth culture (FIG. 20A). The C1 MLD alone is not toxic when expressed in yeast (FIG. 20A), as previously shown^{31,37}.

Distinct from studies in HeLa cells, yeast cells expressing C2A alone were viable when plated on galactose (FIG. 20B). As an alternative verification for the essentiality of C2B in yeast, stop codons were introduced in the yeast expression plasmid at the codons for V3906 and G3948. Similar to expression of C2A alone, cells expressing proteins truncated within C2B also grew under inducing conditions on both plates and in broth (FIG. 20C). Close examination of the plating efficiency of C2A compared to C2B indicates that expression of C2A alone may show a slight growth inhibition on plates or in broth but the effect is modest (FIG. 20B).

Overall, in yeast, distinct from HeLa cells, both C2A and C2B are required for full toxicity although some toxicity is exhibited by C2A alone. The additional requirement for C2B in yeast may reflect a modest difference in the stability of the protein in yeast.

Identification of a C2A Inactivating Mutation by Alanine Scanning Mutagenesis in S. Cerevisiae

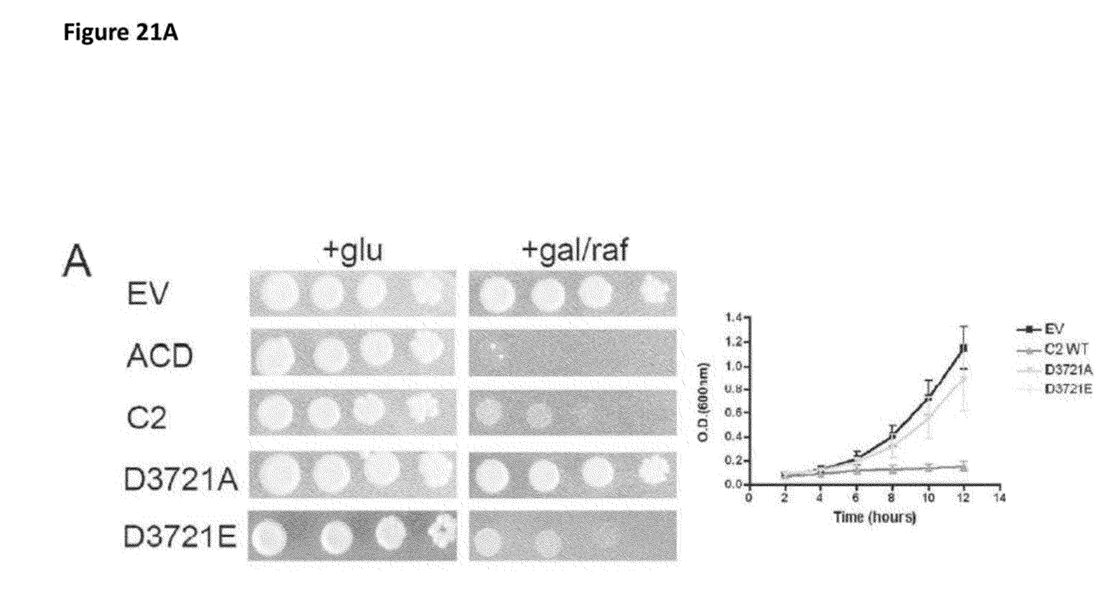

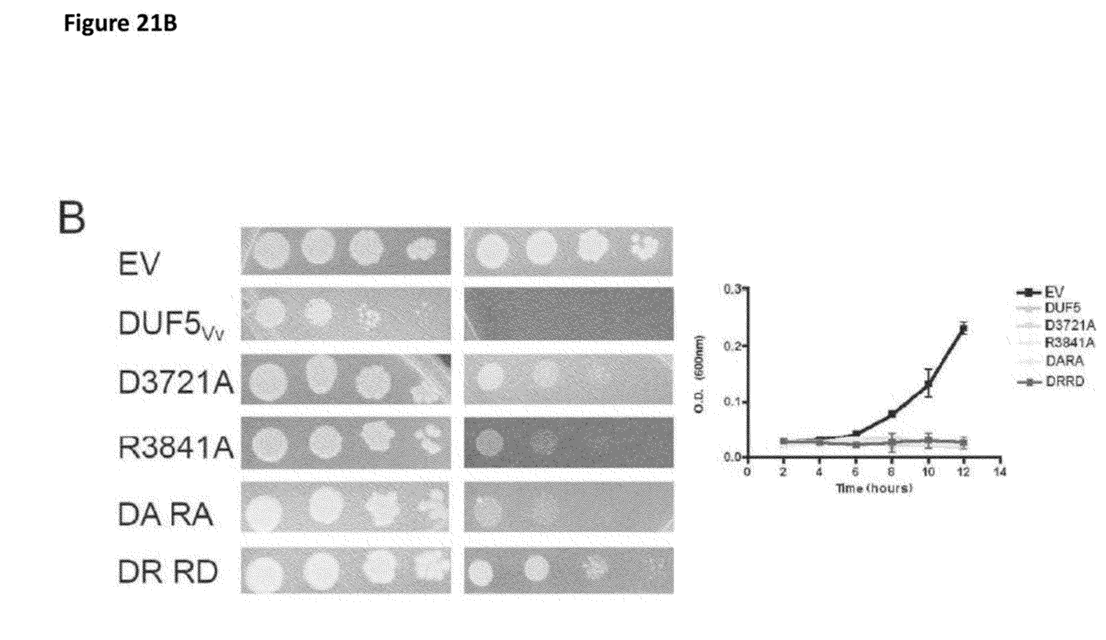

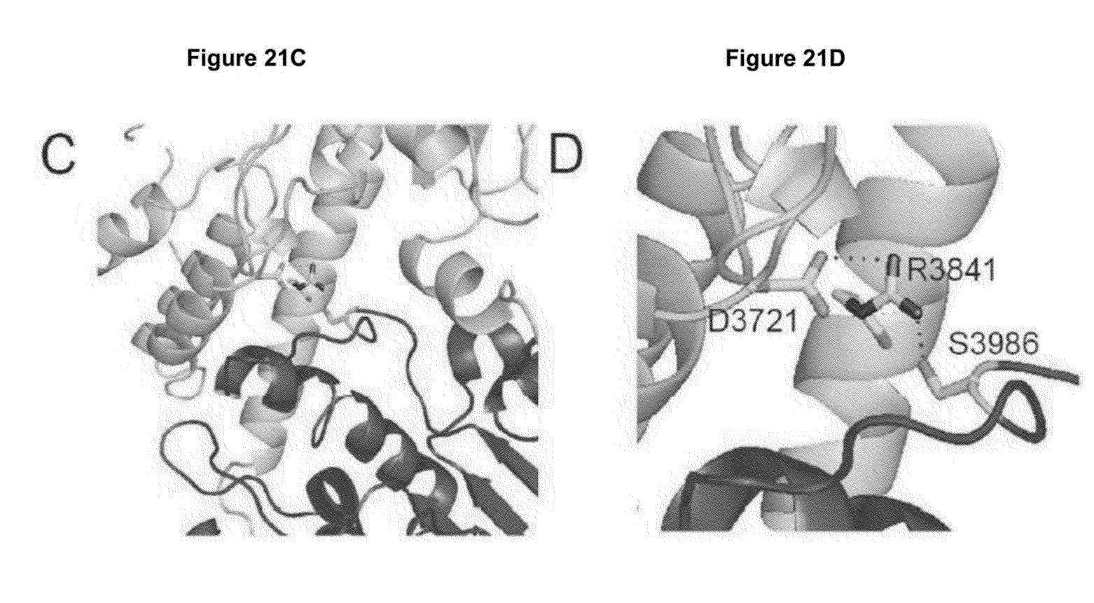

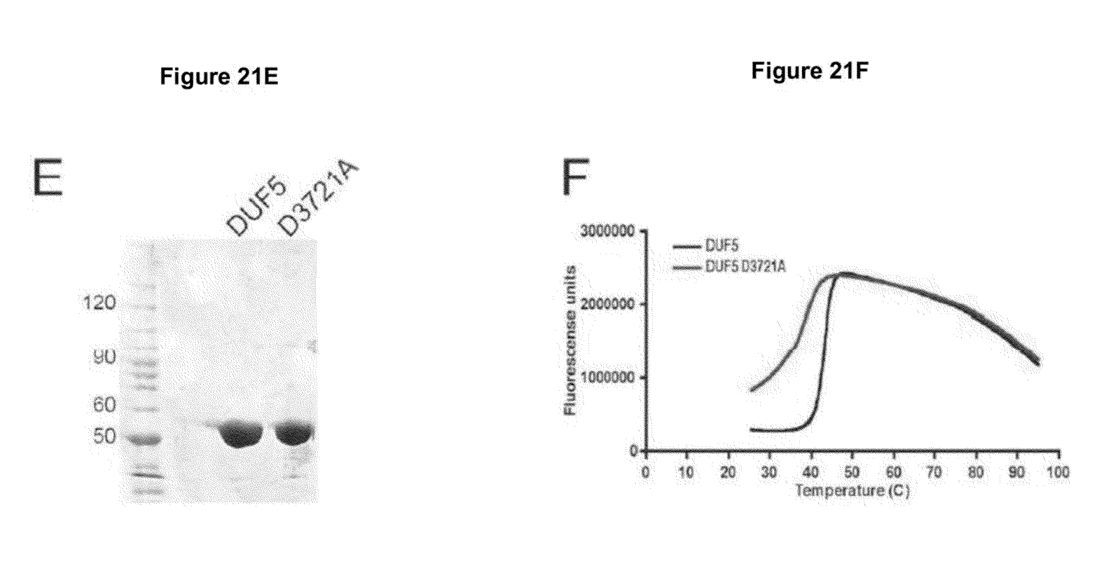

Alanine scanning mutagenesis has proven to be a useful tool to identify critical residues for other of MARTX effector domains^{12,31,38}. Alignment of DUF5 amino acid sequences from 5 MARTX toxins, Plu2400, and PMT showed that there are only 16 residues (8.5%) that are 100% identical across all proteins. If the potentially inactive PMT is excluded, 38 residues (20%) are identical across the remaining 6 effectors (FIG. 21).

To avoid severe toxicity associated with expression of full-length DUF5_{Vv} in yeast, the plasmid for expression of C2 without the MLD was modified by site-directed mutagenesis targeting 65 total residues in C2A (FIG. 21, indicated by asterisks), focusing predominantly on polar residues known to be important for catalysis of other bacterial toxins. Both conserved and non-conserved residues were changed to alanine codons. In addition, nine highly conserved residues in C2B were changed for alanine.

S. cerevisiae transformed with mutagenized plasmids were recovered by growth on glucose and then tested for the ability to grown on galactose. Surprisingly, 73/74 of the mutations did not alter the growth inhibition exhibited by strains expression unaltered C2. The high frequency of mutations showing no relief of toxicity suggests that modest changes to overall structure are not sufficient to overcome the severe toxicity of this protein for yeast, even when the C1 MLD is absent.

Only one mutant, D3721A found within the C2A domain was identified that facilitated growth of yeast expressing C2. By contrast, a more conservative substitution to glutamic acid did not restore growth to yeast (FIG. 21A). D3721 is one of the 16 residues within C2A that is highly conserved in all the DUF5-like proteins, including PMT (FIG. 19).

As an independent verification of the importance of this residue to the function DUF5_{Vv}, the mutation was transferred onto the full-length clone of DUF5_{Vv} for expression in yeast. In this background, the mutation improved growth of yeast under non-inducing conditions to levels near vector control. Under inducing conditions, the plating efficiency was improved 100- to 1000-fold compared to expression of full-length DUF5_{Vv}, although the growth inhibition was not alleviated as shown in liquid culture experiments (FIG. 21B).

Examination of the structural model of DUF5_{Vv} (FIG. 16A) showed that D3721 is present on helix 3 of C2A and makes polar contacts with R3841 on the final helix of C2A just before the start of C2B (FIG. 21C,D). Previous change of R3841 to Ala as part of the screen indicated this residue was not essential in the context of C2. However, in the context of the full-length DUF5_{Vv} that includes the MLD, the phenotype of DUF5_{Vv} R3841A is nearly identical to the phenotype of the D3721A resulting in an improved plating efficiency but poor growth in broth culture (FIG. 21B). This residue is also among the 16 100% identical residues found within C2A (FIG. 19). Combining the D3721A and R3841A resulted in a phenotype identical to that observed for either D3721A or R3841A substitutions alone and did not demonstrate an additive effect. Swapping the aspartate and arginine (D3721R/R3841D) did not improve growth either by plating or broth culture indicating that the potential bridge created by these residues is positional specific.

D3721 and R3841 are Essential for Cytotoxicity

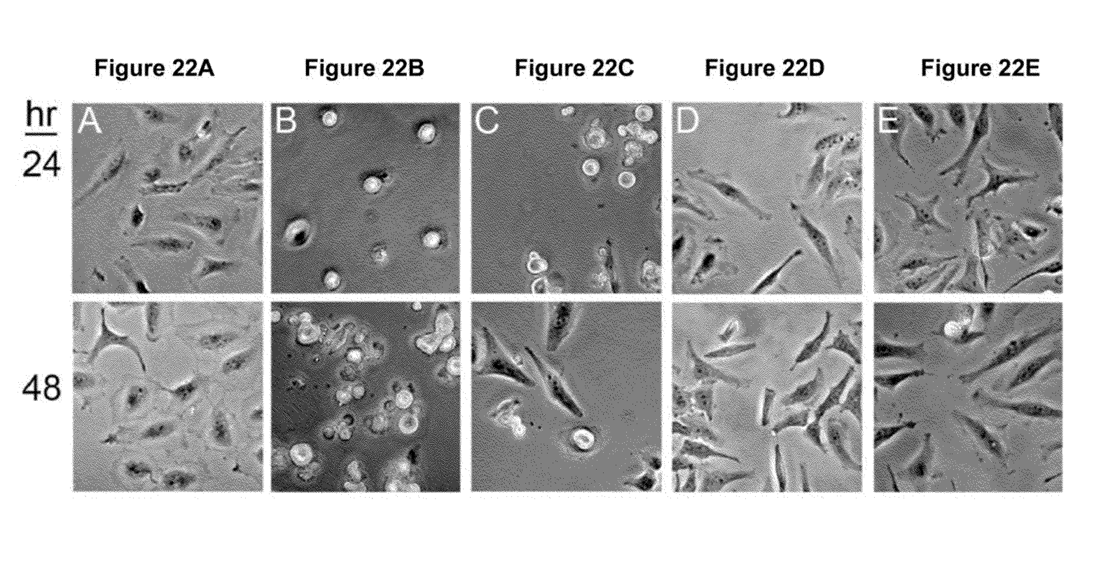

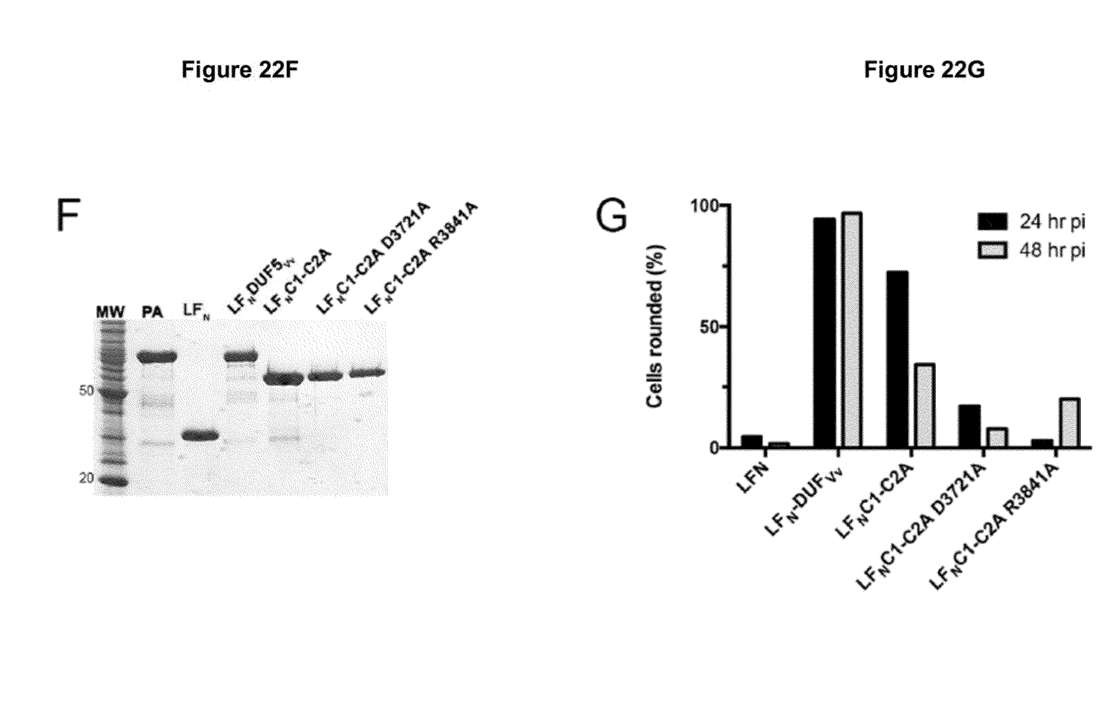

As a final demonstration of the structural requirements for cytoxicity, the D3271A and R3841A mutations were introduced onto the recombinant overexpression plasmid for production of C1-C2A fused to LFN. Proteins were prepared for each mutant from insoluble pellets, urea was reduced to 1.2 M, and the unfolded proteins were delivered to cells by PA. Both mutants lost function in cytotoxicity compared to the similarly prepared unmodified LFNC1-C2A_{Vv} protein (FIG. 22A-E). Assessment of intoxication over time showed that cells treated with PA plus full length LFNDUF5_{Vv} did not recover after 24 h intoxication and nearly 100% of cells remained rounded out to 48 hr. By contrast, ˜50% of cells initially intoxicated with PA plus LFNC1-C2A_{Vv} recovered between 24 and 48 h and returned to normal shape. These data suggest either that C2B carries an additional cytopathic function that prevents recovery of the rounded cells or, more likely, that C2B stabilizes C2A such that the toxin avoids turnover in the cells after successfully inducing cell intoxication. In support of this possibility, fluorescence thermal shift experiments were conducted with full-length recombinant 6×His-DUF5_{Vv} without fusion to LFN (FIG. 21E). This recombinant 6×His-DUF5_{Vv} has a half-maximal melting temperature (Tm) of 43.8° C., while the D3271A substitution lowers Tm by 6.0° C. to 37.8° C. The lower Tm indicates that D3271A causes a structural disturbance that can explain the reduced toxicity seen in yeast and HeLa cells indicating its interaction with R3841 may function to stabilize the protein structure rather than serve as a catalytic residue (FIG. 21F). This is also consistent with the structural model of DUF5 where D3721 is located within the core of the protein, such that a mutation to alanine would cause a disturbance consistent with a drop in Tm and would also account for the higher initial fluorescence seen with DUF5 D3721A than wild type protein.

Discussion

In this study, we undertook a structure-function approach to discover if the V. vulnificus MARTX toxin effector domain DUF5_{Vv} is a cytotoxin accounting for its dramatic effect on virulence in mouse infection studies⁵. The C1_{Vv} subdomain of this protein has been previously shown to localize to anionic membranes, but the function of the C2_{Vv} subdomain at the membrane had not been previously investigated. Here, we demonstrate that DUF5_{Vv} effector domain is cytotoxic to HeLa cells and to yeast resulting in growth inhibition. Further, the cytotoxic activity is localized to its C2A subdomain. In retrospect, mapping the cytotoxicity to the C2A subdomain is surprising because recent computer-based modeling studies of DUF5_{Vv} and related proteins linked the C2B domain to the TIKI/TraB family of proteases leading to the proposal that C2B is a peptidase that functions in signaling^{34,35}. However, we found that any putative protease activity associated with C2B would not contribute to cytotoxicity as complete removal of the subdomain from DUF5_{Vv}-EGFP did not affect cytotoxicity after ectopic expression studies in HeLa cells and expression of C2B-EGFP did not cause any observable effect in HeLa cells. Further the computer-based analysis indicated that C2B residue H3902 would be essential for peptidase activity, but this residue was among those modified during expression in yeast that did not restore the ability of yeast to grow (FIG. 19). These findings convincingly link the cytotoxic effect of DUF5_{Vv} to its C2A subdomain; however, we cannot exclude that the C2B in addition to C2A could modify cell biological processes in manner that does not affect cell viability or morphology during MARTX intoxication and that DUF5_{Vv} itself is a multifunctional effector domain.

The remainder of the study focused on identification of residues within C2A_{Vv} that are essential for its cytotoxicity. Growth of yeast expressing C2_{Vv} was used as a method to screen point mutations to identify those that would overcome the severe toxicity in yeast, a highly stringent phenotype generally indicative of an essential residue. The screen revealed a single essential Asp that initially was considered as a possible catalytic residue. However, the absence of additional residues in C2A_{Vv} that would be predicted to form a catalytic site along with the finding that other highly conserved Gly, Pro, Tyr, Phe, Leu, and Ala are not essential suggests this subdomain functions by binding to a target protein rather than by covalent modification. The ability of the residue to tolerate substitution to the more structurally conservative glutamic acid also indicates this is not likely an aspartyl protease. We further found that the D3721A substitution reduced the Tm of the DUF5_{Vv} indicating structural destabilization as opposed to loss of catalytic function.

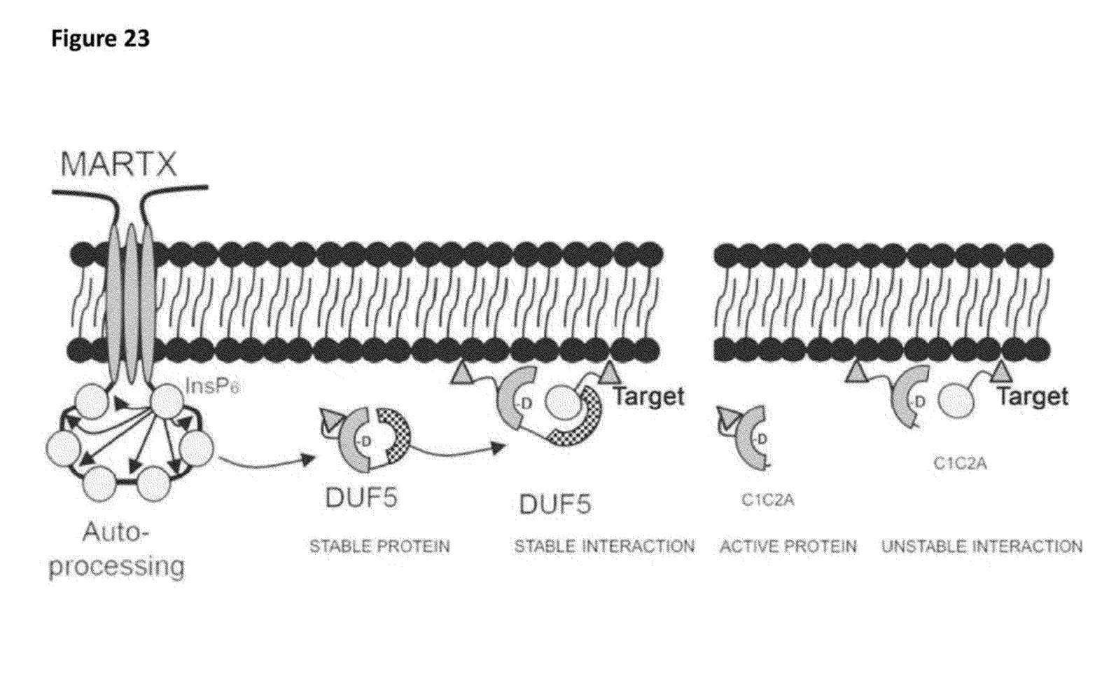

This stabilization may be due to its association with R3841 to retain optimal folding of the face that binds to the target protein or to serve as a switch to facilitate a change in the DUF5_{Vv} structural conformation upon binding of C1_{Vv} to the membrane (FIG. 21D). The role as a switch in the context of membrane binding is particularly interesting since reduced toxicity due to R3841A was observed only in the context of the C1_{Vv} membrane localization domain in both yeast and HeLa cells. The contact between D3721 and R3841 could affect the conformation at the interface between C2A_{Vv} and C2B_{Vv} since R3841 that makes polar contacts with D3721 also contacts a 53986 in an unstructured loop of C2B. In other DUF5 homologues, the Ser is replaced by a Thr. Further, this Ser is absent from PMT, although Ser residues are localized nearby in this otherwise poorly conserved regions between PMT and DUF5_{Vv}. Thus, it is intriguing to speculate that C2B_{Vv} could function as a stabilization subdomain for C2A_{Vv} with D3721 and R3841 functioning as part of the conformational switch to open up a binding site for the cellular target of C2A (FIG. 23).

A final component not addressed in this study is the biochemical mechanism or activity of DUF5_{Vv} and DUF5_{Ah}. While two residues, D3721 and R3841 were found to be essential for rounding of mammalian cells by DUF5_{Vv}, this discovery does not as yet inform the biochemical or cell biological activity that results in cell rounding. This is particularly true since residues shown to be essential for DUF5_{Vv} (D3721 and R3841) and conserved in DUF5 (D3215 and R33e5) are also conserved in PMT (as D720 and R861). Given that PMT is not able to round cells similar to DUF5_{Vv} and DUF5Ah, we can only speculate that surrounding residues not conserved in PMT also contribute to the appropriate structure for DUF5_{Vv} and DUF5 allowing these proteins but not PMT to properly interact with cellular components. Despite not yet directly demonstrating the biochemical or cell biological activity of the MARTX DUF5 effector domains, this study has provided numerous useful tools and reagents for these on-going studies but likewise reveals how identification of the cellular target could potentially be problematic. We found that the cytotoxicity is associated with C2A. However, this subdomain is highly toxic when ectopically overexpressed, which presents difficulties in identifying the target protein by common affinity precipitation techniques. A catalytically inactive variant is often highly useful to trap targets by affinity precipitation methods, but we found that the only inactive substitution also affects structural integrity and likely no longer binds its target in vivo. Our findings here that yeast is also affected by DUF5_{Vv} does open the possibility that yeast-based genetic approaches could be very helpful to identify the target and these studies are currently ongoing.

### REFERENCES

1. Satchell K J. Structure and function of MARTX toxins and other large repetitive RTX proteins. Annual review of microbiology 2011; 65:71-90.
2. Egerer M, Satchell K J. Inositol hexakisphosphate-induced autoprocessing of large bacterial protein toxins PLoS Pathog 2010; 6(7):e1000942.
3. Prochazkova K, Shuvalova L A, Minasov G, Voburka Z, Anderson W F, Satchell K J. Structural and molecular mechanism for autoprocessing of MARTX Toxin of Vibrio cholerae at multiple sites. J Biol Chem 2009; 284:26557-26568.
4. Shen A, Lupardus P J, Albrow V E, Guzzetta A, Powers J C, Garcia K C, Bogyo M. Mechanistic and structural insights into the proteolytic activation of Vibrio cholera MARTX toxin. Nat Chem Biol 2009; 5(7):469-478.
5. Kwak J S, Jeong H G, Satchell K J. Vibrio vulnificus rtxA1 gene recombination generates toxin variants with altered potency during intestinal infection. Proceedings of the National Academy of Sciences of the United States of America 2011; 108(4):1645-1650.
6. Roig F J, Gonzalez-Candelas F, Amaro C. Domain organization and evolution of multifunctional autoprocessing repeats-in-toxin (MARTX) toxin in Vibrio vulnificus. Appl Environ Microbiol 2011; 77(2):657-668.
7. Cordero C L, Kudryashov D S, Reisler E, Satchell K J. The actin cross-linking domain of the Vibrio cholerae RTX toxin directly catalyzes the covalent cross-linking of actin. J Biol Chem 2006; 281(43):32366-32374.
8. Kudryashov D S, Durer Z A, Ytterberg A J, Sawaya M R, Pashkov I, Prochazkova K, Yeates T O, Loo R R, Loo J A, Satchell K J, Reisler E. Connecting actin monomers by isopeptide bond is a toxicity mechanism of the Vibrio cholerae MARTX toxin. Proceedings of the National Academy of Sciences of the United States of America 2008; 105(47):18537-18542.
9. Fullner K J, Mekalanos J J. In vivo covalent crosslinking of actin by the RTX toxin of Vibrio cholerae. EMBO J 2000; 19:5315-5323.
10. Sheahan K L, Cordero C L, Satchell K J. Identification of a domain within the multifunctional Vibrio cholerae RTX toxin that covalently cross-links actin. Proc Natl Acad Sci USA 2004; 101(26):9798-9803.
11. Sheahan K L, Satchell K J. Inactivation of small Rho GTPases by the multifunctional RTX toxin from Vibrio cholerae. Cell Microbiol 2007; 9(5):1324-1335.
12. Ahrens S, Geissler B, Satchell K J. Identification of a His-Asp-Cys catalytic triad essential for function of the Rho inactivation domain (RID) of Vibrio cholerae MARTX toxin. The Journal of biological chemistry 2013; 288(2):1397-1408.
13. Ziolo K. J. J H, Kwak J. S., Yang S., Lavker R. M. and Satchell K. J. F. Vibrio vulnificus biotype 3 MARTX toxin is an adenylate cyclase toxin essential for virulence in mice. Infection and Immunity 2014.
14. Satchell K J. MARTX: Multifunctional-Autoprocessing RTX Toxins. Infect Immun 2007; 75:5079-5084.
15. Kitadokoro K, Kamitani S, Miyazawa M, Hanajima-Ozawa M, Fukui A, Miyake M, Horiguchi Y. Crystal structures reveal a thiol protease-like catalytic triad in the Cterminal region of Pasteurella multocida toxin. Proceedings of the National Academy of Sciences of the United States of America 2007; 104(12):5139-5144.
16. Kamitani S, Kitadokoro K, Miyazawa M, Toshima H, Fukui A, Abe H, Miyake M, Horiguchi Y. Characterization of the membrane-targeting C1 domain in Pasteurella multocida toxin. The Journal of biological chemistry 2010; 285(33):25467-25475.
17. Geissler B, Tungekar R, Satchell K J. Identification of a conserved membrane localization domain within numerous large bacterial protein toxins Proceedings of the National Academy of Sciences of the United States of America 2010; 107(12):5581-5586.
18. Brothers M C, Geissler B, Hisao G S, Satchell K J, Wilson B A, Rienstra C M. Backbone and side-chain resonance assignments of the membrane localization domain from Pasteurella multocida toxin. Biomolecular NMR assignments 2014; 8(1):221-224.
19. Brothers M C, Geissler B, Hisao G S, Wilson B A, Satchell K J, Rienstra C M. Backbone and side-chain assignments of an effector membrane localization domain from Vibrio vulnificus MARTX toxin. Biomolecular NMR assignments 2013.
20. Pullinger G D, Sowdhamini R, Lax A J. Localization of functional domains of the mitogenic toxin of Pasteurella multocida. Infect Immun 2001; 69(12):7839-7850.
21. Aminova L R, Luo S, Bannai Y, Ho M, Wilson B A. The C3 domain of Pasteurella multocida toxin is the minimal domain responsible for activation of Gq-dependent calcium and mitogenic signaling. Protein Sci 2008; 17(5):945-949.
22. Orth J H, Preuss I, Fester I, Schlosser A, Wilson B A, Aktories K. Pasteurella multocida toxin activation of heterotrimeric G proteins by deamidation. Proc Natl Acad Sci USA 2009.
23. Orth J H, Fester I, Siegert P, Weise M, Lanner U, Kamitani S, Tachibana T, Wilson B A, Schlosser A, Horiguchi Y, Aktories K. Substrate specificity of Pasteurella multocida toxin for alpha subunits of heterotrimeric G proteins. FASEB J 2013; 27(2):832-842.
24. ffrench-Constant R, Waterfield N, Daborn P, Joyce S, Bennett H, Au C, Dowling A, Boundy S, Reynolds S, Clarke D. Photorhabdus: towards a functional genomic analysis of a symbiont and pathogen. FEMS Microbiol Rev 2003; 26(5):433-456.
25. Wilkinson P, Waterfield N R, Crossman L, Corton C, Sanchez-Contreras M, Vlisidou I, Barron A, Bignell A, Clark L, Ormond D, Mayho M, Bason N, Smith F, Simmonds M, Churcher C, Harris D, Thompson N R, Quail M, Parkhill J, Ffrench-Constant R H. Comparative genomics of the emerging human pathogen Photorhabdus asymbiotica with the insect pathogen Photorhabdus luminescens. BMC Genomics 2009; 10:302.
26. Altschul S F, Gish W, Miller W, Myers E W, Lipman D J. Basic local alignment search tool. J Mol Biol 1990; 215(3):403-410.
27. Soding J, Biegert A, Lupas A N. The HHpred interactive server for protein homology detection and structure prediction. Nucleic acids research 2005; 33 (Web Server issue):W244-248.
28. Sali A, Potterton L, Yuan F, van Vlijmen H, Karplus M. Evaluation of comparative protein modeling by MODELLER. Proteins 1995; 23(3):318-326.
29. Spyres L M, Qa'Dan M, Meader A, Tomasek J J, Howard E W, Ballard J D. Cytosolic delivery and characterization of the TcdB glucosylating domain by using a heterologous protein fusion. Infect Immun 2001; 69(1):599-601.
30. Stols L, Gu M, Dieckman L, Raffen R, Collart F R, Donnelly M I. A new vector for highthroughput, ligation-independent cloning encoding a tobacco etch virus protease cleavage site. Protein Expr Purif 2002; 25(1):8-15.
31. Geissler B, Bonebrake A, Sheahan K L, Walker M E, Satchell K J. Genetic determination of essential residues of the Vibrio cholerae actin cross-linking domain reveals functional similarity with glutamine synthetases. Molecular microbiology 2009; 73(5):858-868.
32. Busch C, Orth J, Djouder N, Aktories K. Biological activity of a C-terminal fragment of Pasteurella multocida toxin. Infect Immun 2001; 69(6):3628-3634.
33. Baldwin M R, Lakey J H, Lax A J. Identification and characterization of the Pasteurella multocida toxin translocation domain. Molecular microbiology 2004; 54(1):239-250.
34. Bazan J F, Macdonald B T, He X. The TIKI/TraB/PrgY family: a common protease fold for cell signaling from bacteria to metazoa? Dev Cell 2013; 25(3):225-227.
35. Sanchez-Pulido L, Ponting C P. Tiki, at the head of a new superfamily of enzymes. Bioinformatics 2013; 29(19):2371-2374.
36. Wesche J, Elliott J L, Falnes P O, Olsnes S, Collier R J. Characterization of membrane translocation by anthrax protective antigen. Biochemistry 1998; 37(45):15737-15746.
37. Geissler B, Ahrens S, Satchell K J. Plasma membrane association of three classes of bacterial toxins is mediated by a basic-hydrophobic motif. Cellular microbiology 2012; 14(2):286-298.
38. Prochazkova K, Satchell K J. Structure-function analysis of inositol hexakisphosphateinduced autoprocessing of the Vibrio cholerae multifunctional autoprocessing RTX toxin. J Biol Chem 2008; 283(35):23656-23664.

### Example 3

### Site-Specific Processing of Ras and Rap1 Switch I by a MARTX Toxin Effector Domain

Reference is made Antic et al., Nat. Commun. 2015 Jun. 8; 6:7396, the content of which is incorporate herein by reference in its entirety.

Abstract

Ras (Rat sarcoma) protein is a central regulator of cell growth and proliferation. Mutations in the RAS gene are known to occur in human cancers and have been shown to contribute to carcinogenesis. In this study, we show that the multifunctional-autoprocessing repeats-intoxin (MARTX) toxin-effector domain DUF5_{Vv} from Vibrio vulnificus to be a site-specific endopeptidase that cleaves within the Switch 1 region of Ras and Rap1. DUF5_{Vv} processing of Ras, which occurs both biochemically and in mammalian cell culture, inactivates ERK1/2, thereby inhibiting cell proliferation. The ability to cleave Ras and Rap1 is shared by DUF5_{Vv} homologues found in other bacteria. In addition, DUF5_{Vv} can cleave all Ras isoforms and KRas with mutations commonly implicated in malignancies. Therefore, we speculate that this new family of Ras/Rap1A-specific endopeptidases (RRSPs) has potential to inactivate both wild-type and mutant Ras proteins expressed in malignancies.

Introduction

Rat sarcoma (Ras) oncoprotein is a small GTPase ubiquitous in eukaryotic cells and is a critical node that coordinates incoming signals and subsequently activates downstream target proteins. These targets include rapidly accelerated fibrosarcoma kinase (Raf), phosphatidylinositol-4,5-bisphosphate 3-kinase and mitogen-activated protein kinase (MAPK), which ultimately induces expression of genes directing cell proliferation, differentiation and survival. Regulation of Ras enzymatic activity is achieved by cycling between an inactive (GDP-bound) state and an active (GTP-bound) state. On activation, conformational changes in the Ras protein structure trigger Ras downstream signalling cascades by binding specific protein effectors¹⁻⁴. Mutations in Ras proto-oncogenes are found in 9-30% of all human malignancies. In addition, Ras point mutations, which are observed at residues G12 and G13 in the P-loop and at Q61 in the Switch II region, are the most common mutations in human malignancies and are present in 98% of pancreatic ductal adenocarcinomas, 53% of colorectal adenocarcinomas and 32% of lung adenocarcinomas⁵⁻⁷. However, effective targeting of Ras has been very difficult and is considered a critical roadblock on the path towards generating new therapeutics against intractable human cancers⁸⁻¹². Despite the potential of Ras proteins as therapeutic targets, there are no inhibitors for any of the three main human isoforms—HRas, KRas and NRas—or their constitutively activated mutant forms⁸⁻¹¹.

From a microbial pathogenesis perspective, activation of Ras is central to cellular detection of bacterial lipopolysaccharide and other pathogen-associated molecular patterns resulting in activation of innate immune defenses¹³. Although several bacterial toxins are known to target Ras by posttranslational modification to circumvent this important host response to infection, to date none have been shown to be highly specific for Ras¹⁴⁻¹⁶.

Multifunctional-autoprocessing repeats-in-toxin (MARTX) toxins proteins are large composite-secreted bacterial protein toxins that translocate across the eukaryotic cell plasma membrane and deliver multiple cytopathic and cytotoxic effector proteins from a single holotoxin by autoprocessing^{17,18}. In our previous work, we showed that the most highly virulent strains of the sepsis-causing pathogen V. vulnificus produce a 5,206-amino acid (aa) MARTX toxin with an extra effector domain termed DUF5_{Vv}, for the domain of unknown function in the 5^{th} position¹⁹. In fact, bacterial strains that produce a MARTX toxin with DUF5_{Vv} are found to be 10- to 50-fold more virulent in mice than strains that produce a MARTX toxin without DUF5_{Vv} (ref 19). These data directly connect DUF5_{Vv} with increased virulence during infection.

The 509-aa DUF5_{Vv} effector domain of the MARTX toxin was highly cytotoxic when ectopically expressed as a fusion to green fluorescent protein (GFP), resulting in rounding and shrinkage of cells²⁰. Structural and functional bioinformatics studies have demonstrated that DUF5_{Vv} is comprises two subdomains^{20,21}. The amino-terminal C1 subdomain is a four-helix bundle that mediates localization to the plasma membrane by binding anionic phospholipids^{21,22}. The carboxy-terminal C2 subdomain confers the cell rounding activity²⁰. Moreover, DUF5_{Vv}-C2 was found to inhibit growth when conditionally overexpressed in Saccharomyces cerevisiae²⁰.

In this study, we used a combination of genetic, cell biological and biochemical strategies to probe the mechanism of action of the C2 subdomain, to understand the connection of DUF5_{Vv} to both cytotoxicity and increased virulence of the pathogen. We find that DUF5_{Vv} site-specifically processes both Ras and the closely related small GTPase Rap1. Both proteins are critical for activation of the innate immune response during infection, which explains the crucial role of this effector domain in the increased virulence of V. vulnificus strains that have DUF5_{Vv} As Ras is also important for cell proliferation in carcinogenesis, this enzyme could potentially be developed as a treatment for various types of tumours.

Results

DUF5_{Vv} Causes ERK1/2 Dephosphorylation.

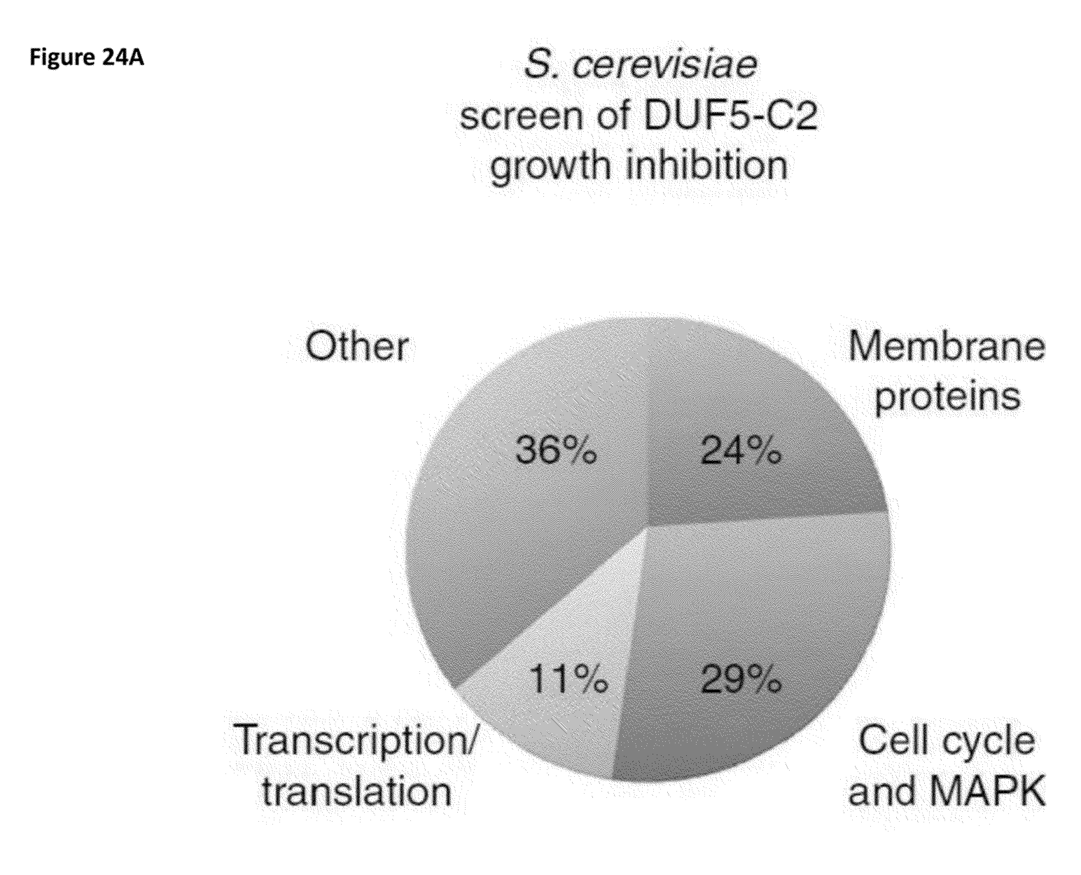

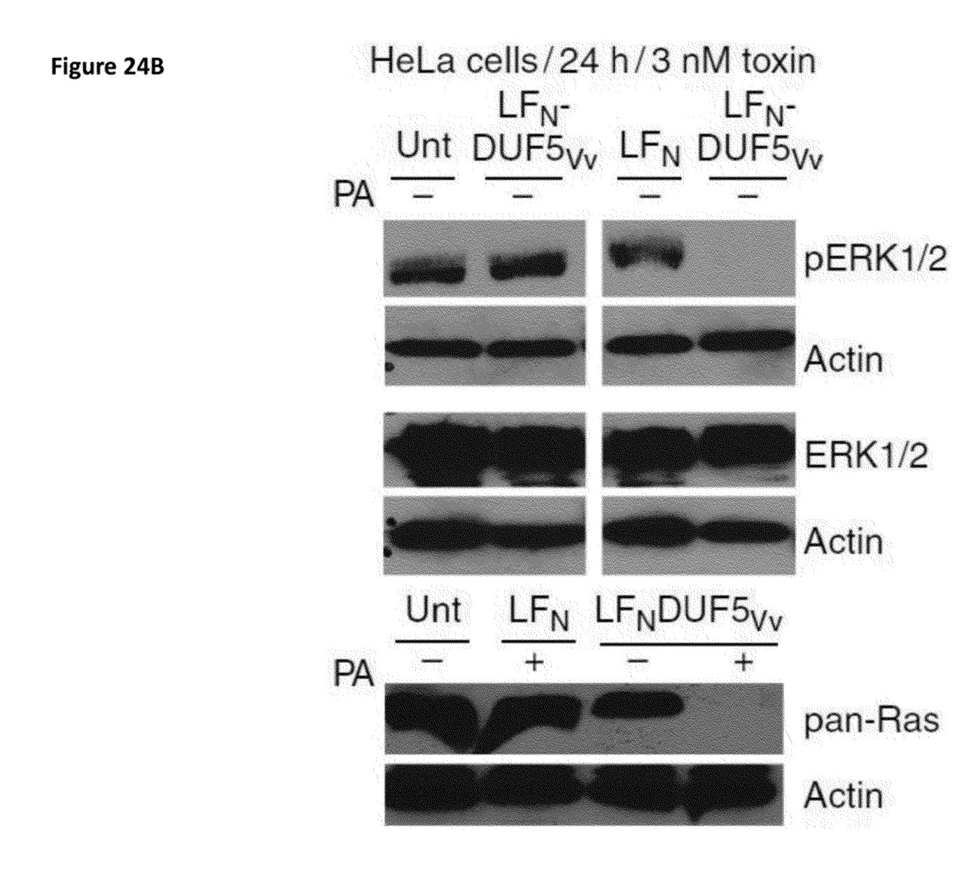

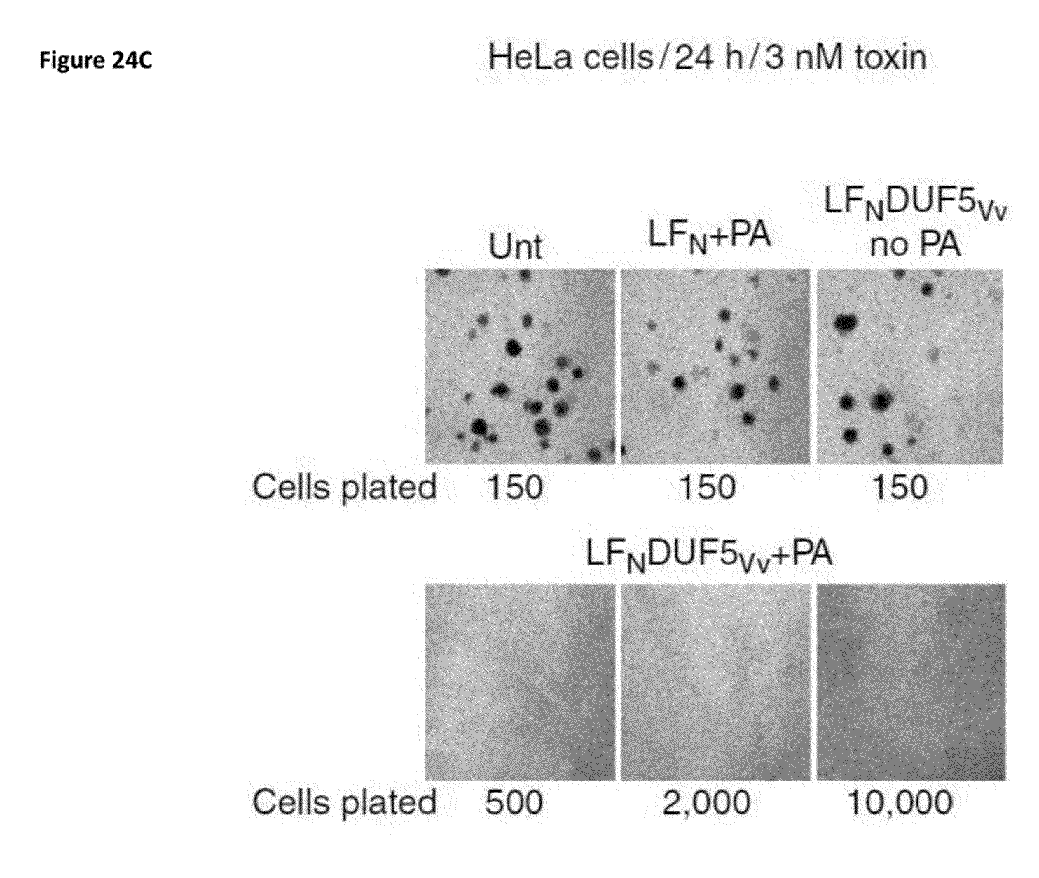

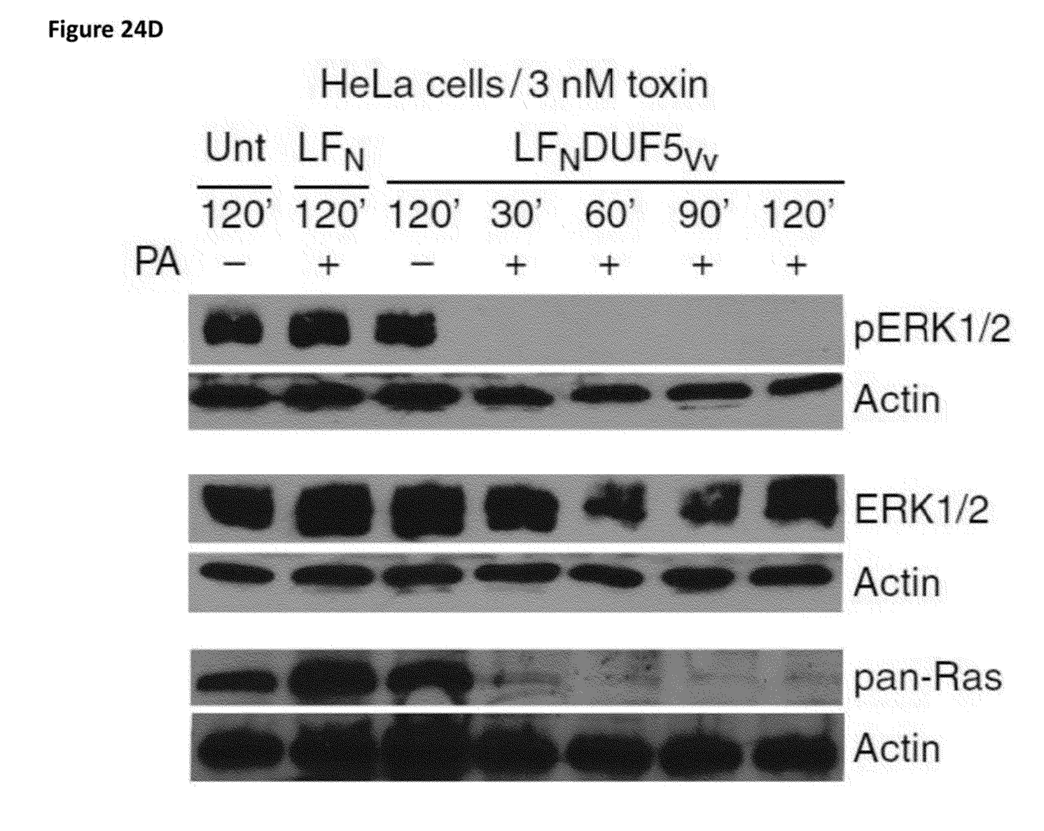

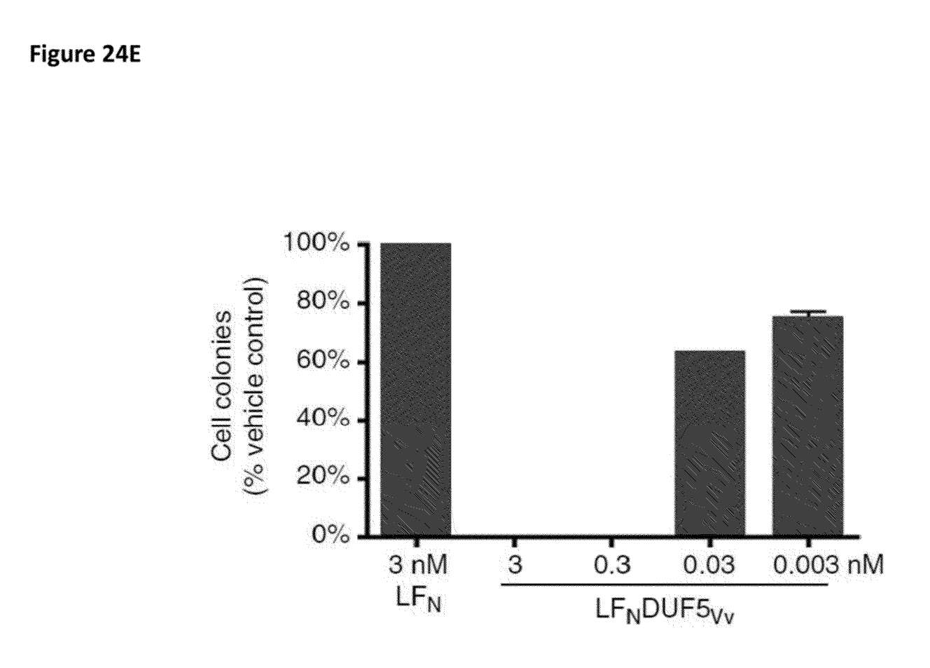

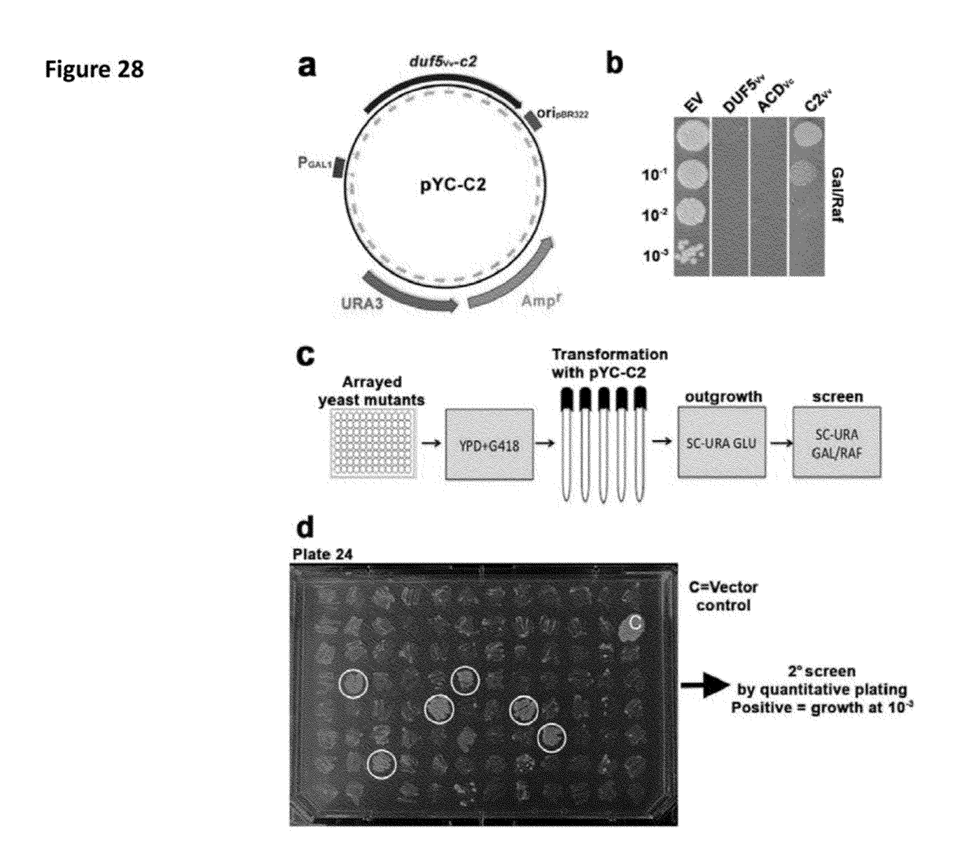

Previously we showed that DUF5_{Vv}-C2 is cytotoxic when ectopically expressed in cells²⁰. As a strategy to identify molecular targets accounting for this cytotoxicity²⁰, a genome-wide, arrayed, non-essential gene deletion library was screened for yeast strains that survived enforced expression of C2 (FIG. 28). Of 4,709 yeast strains screened, 3.6% formed colonies on plates containing the inducer galactose, indicating that the yeast gene disruption suppressed C2-dependent growth inhibition. The hits were categorized based on information in the Saccharomyces Genome Database23. Eleven percent of the mutant yeast strains that overcame growth inhibition due to DUF5_{Vv}-C2 expression harboured deletions in genes for transcription and/or translation. These mutations probably reduce DUF5_{Vv}-C2 expression, accounting for suppression of growth inhibition. Twenty-four percent of the recovered yeast strains had defects affecting membrane or membrane proteins, possibly causing suppression of cytotoxicity due to the absence of the cellular target at the membrane (FIG. 24A).

Among the remaining hits, nearly half were connected to MAPKs or processes they regulate. Therefore, it was postulated that mammalian MAPK p38 and ERK1/2 could have altered activity during exposure of cells to DUF5_{Vv}. We have previously demonstrated that the cytotoxic activity of DUF5_{Vv} can be isolated away from the large MARTX by fusing DUF5_{Vv} to the N terminus of anthrax toxin lethal factor (LFNDUF5_{Vv}) and subsequently delivering the fusion protein to cells in culture using anthrax toxin protective antigen (PA20). Therefore, we used this system to test for changes in MAPK signalling dependent on exposure of cells to DUF5_{Vv}.

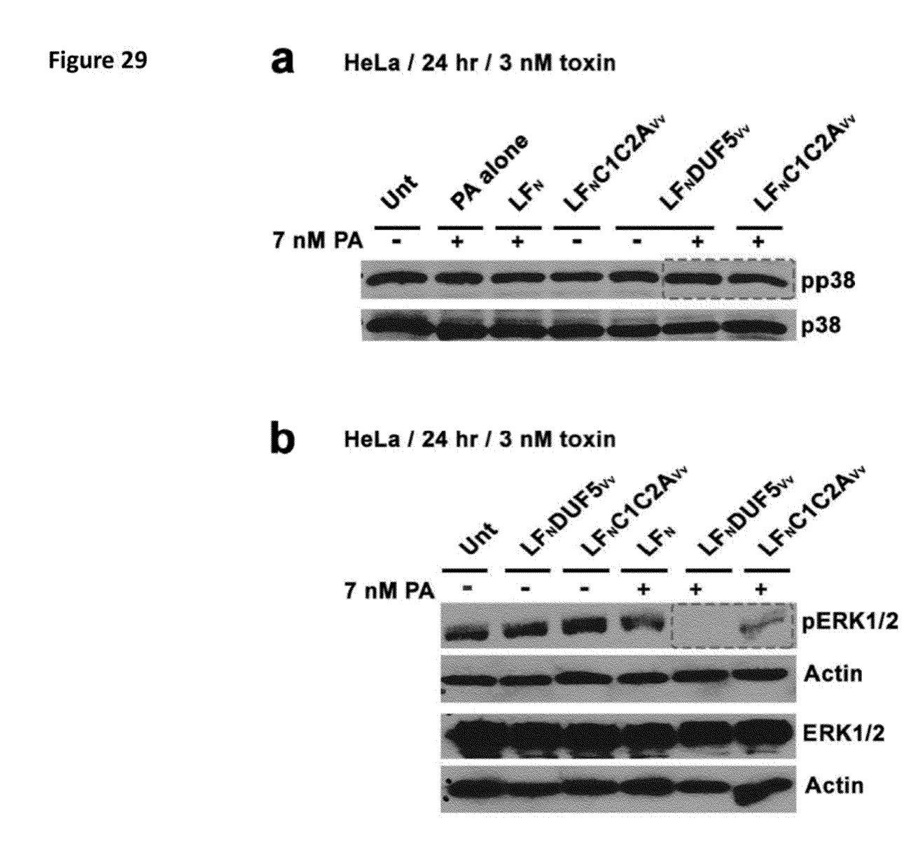

HeLa cervical carcinoma cells constitutively produce high levels of phospho-p38 and phospho-ERK1/2 (pERK1/2), making these cells an ideal model system to determine the underlying mechanism by which DUF5_{Vv} interferes with MAPK signaling (FIG. 29). For cells intoxicated with LFNDUF5_{Vv} in combination with PA for 24 h, no change in levels of phosphop³⁸ was observed (FIG. 29a). However, there was a marked absence of pERK1/2 in HeLa cells treated with LFNDUF5_{Vv}+PA (FIG. 24B and FIG. 29a). In addition, the first 276 aa of DUF5_{Vv}, corresponding to the C1 membrane-targeting subdomain and the first 186 of C2 (C1C2A_{Vv}), were sufficient to reduce pERK1/2 levels (FIG. 29b), consistent with previous results showing that C1C2A_{Vv} is sufficient for cell rounding activity²⁰. Thus, the yeast screen and subsequent studies in HeLa cells revealed that DUF5_{Vv} modulates the activation state of ERK1/2 without affecting p38.

Ras Depletion by DUF5_{Vv} Inhibits Cell Division.

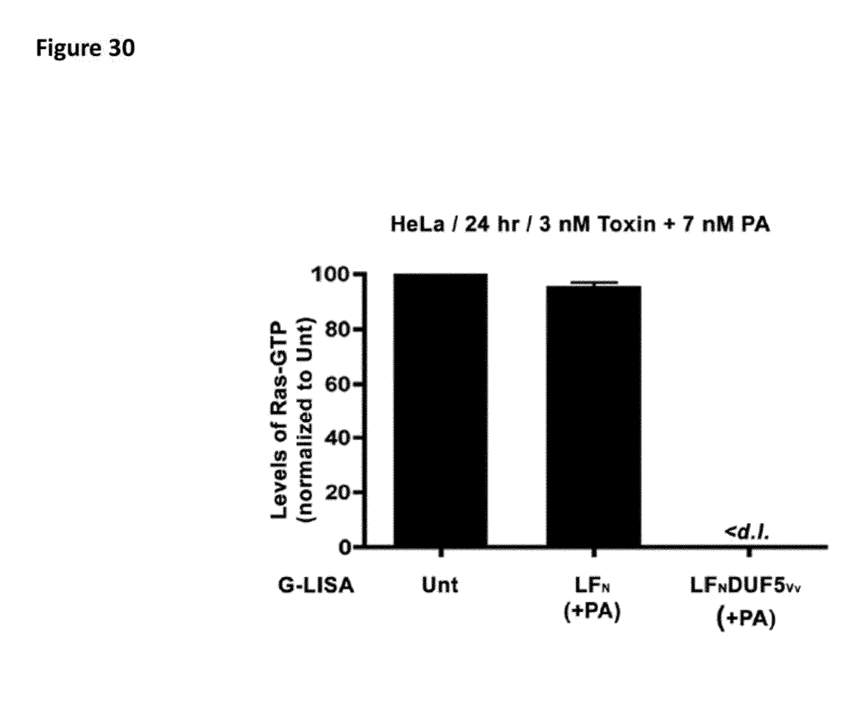

Owing to its C1 membrane-targeting subdomain, DUF5_{Vv} is exclusively present at the plasma membrane²¹; hence, inactivation of membrane localized Ras GTPases that control activation of ERK1/2 (refs 24,25) seemed a plausible mechanism for DUF5_{Vv} dependent ERK1/2 dephosphorylation. Active Ras (GTP-bound) was probed using a G-LISA assay, where wells are coated with a Ras GTP-binding protein domain. Surprisingly, active Ras was undetectable in cell lysates intoxicated with LFNDUF5_{Vv}+PA, suggesting that Ras was exclusively in the inactive, GDP-bound state (FIG. 30). This result initially suggested that DUF5_{Vv} affects levels of active Ras-GTP. However, additional control experiments revealed that Ras protein itself was undetectable in cell lysates, as measured by immunoblotting with a monoclonal anti-RAS10 antibody that detects all isoforms of Ras26, including KRas, HRas and NRas (FIG. 24b). This experiment shows that DUF5_{Vv} directly targets the Ras protein rather than indirectly affecting its regulation.

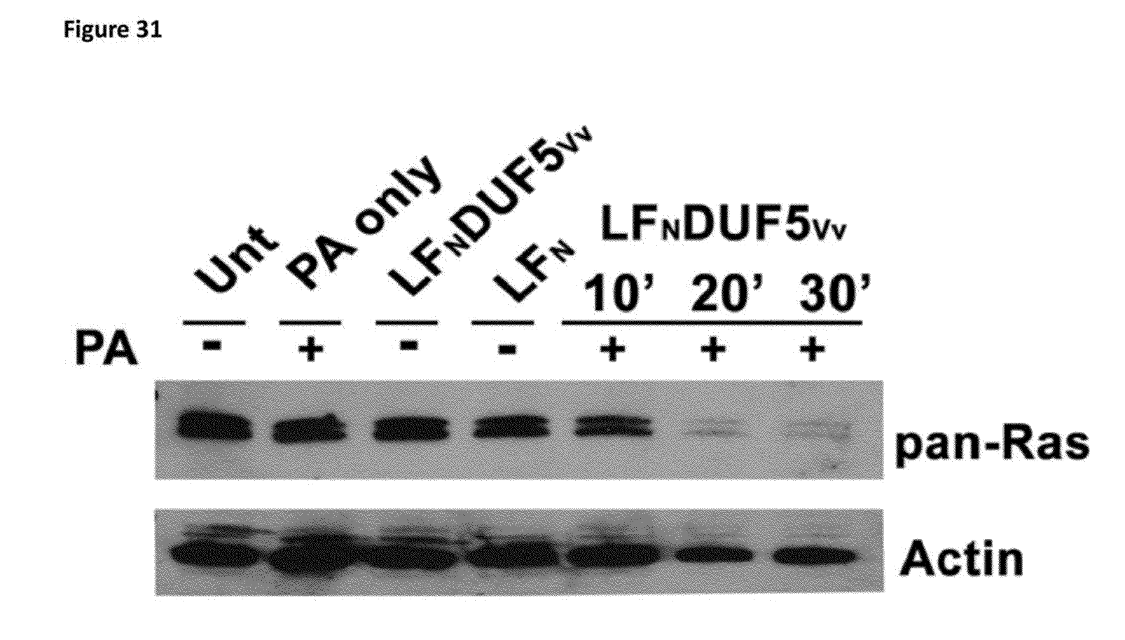

If Ras and pERK1/2 are truly absent from DUF5_{Vv}-treated cells, proliferation should be inhibited in intoxicated samples. To measure disruption in cell proliferation due to the inhibition of the Ras-ERK pathway, the toxin was removed by washing, and treated cells were plated and resulting colonies counted after a 14-day incubation period. HeLa cells intoxicated for 24 h did not produce colonies even when plated at almost 70-fold higher seeding densities than control-treated cells (FIG. 24C). Examination of ERK1/2 and Ras inactivation over time revealed that exposure of cells to 3 nM LFNDUF5_{Vv} for only 30 min was sufficient for nearly 100% inactivation (FIG. 24D and FIG. 31). In addition, exposure of cells to LFNDUF5_{Vv} concentrations as low as 30 μM for 1 h was sufficient to significantly decrease cell proliferation (FIG. 24E). Overall, these studies reveal that DUF5_{Vv} directly targets Ras, resulting in loss of ERK1/2 phosphorylation and cell proliferation.

Ras is Cleaved at the N Terminus in DUF5_{Vv}-Treated Cells.

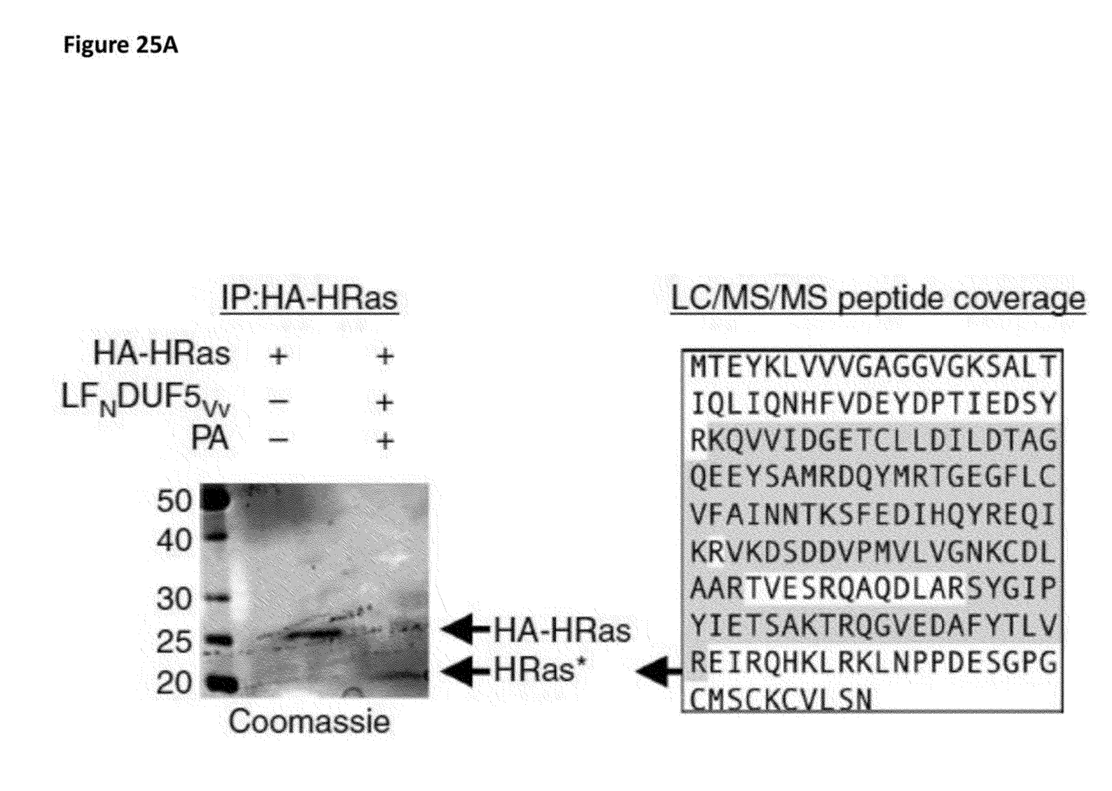

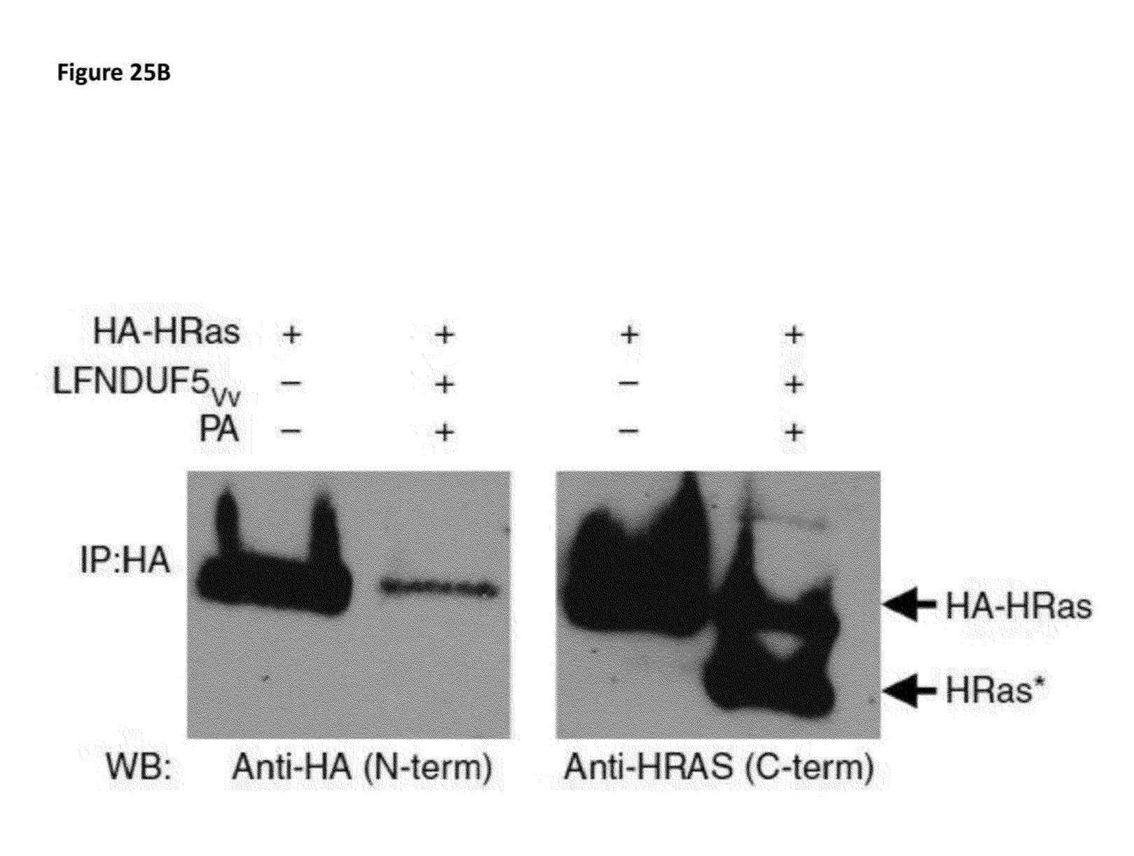

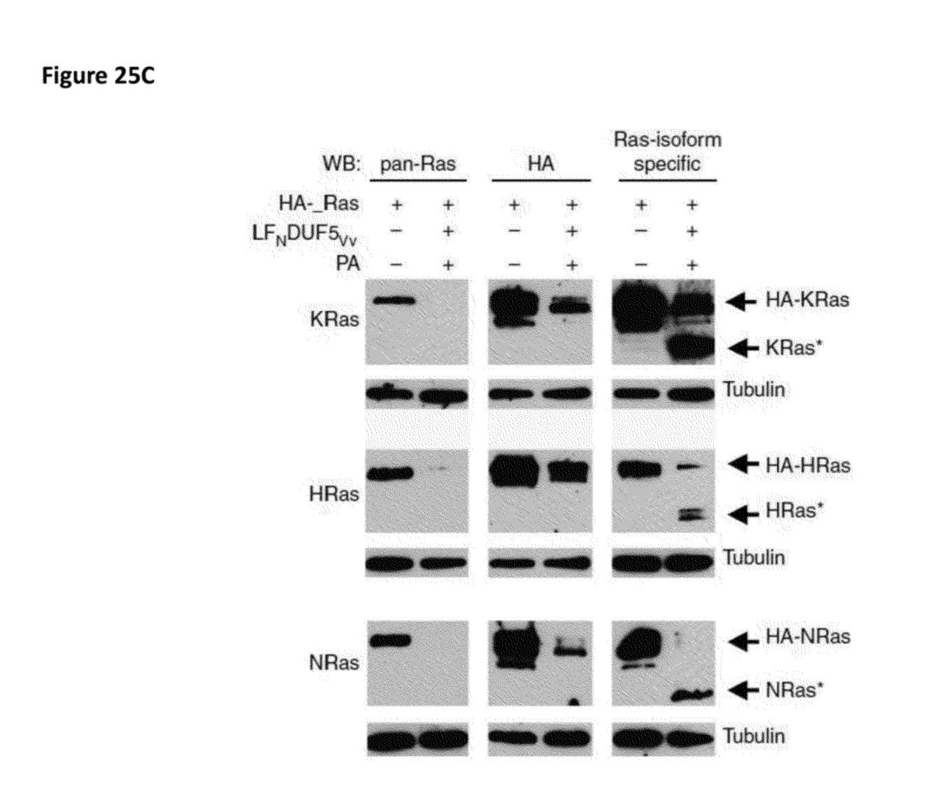

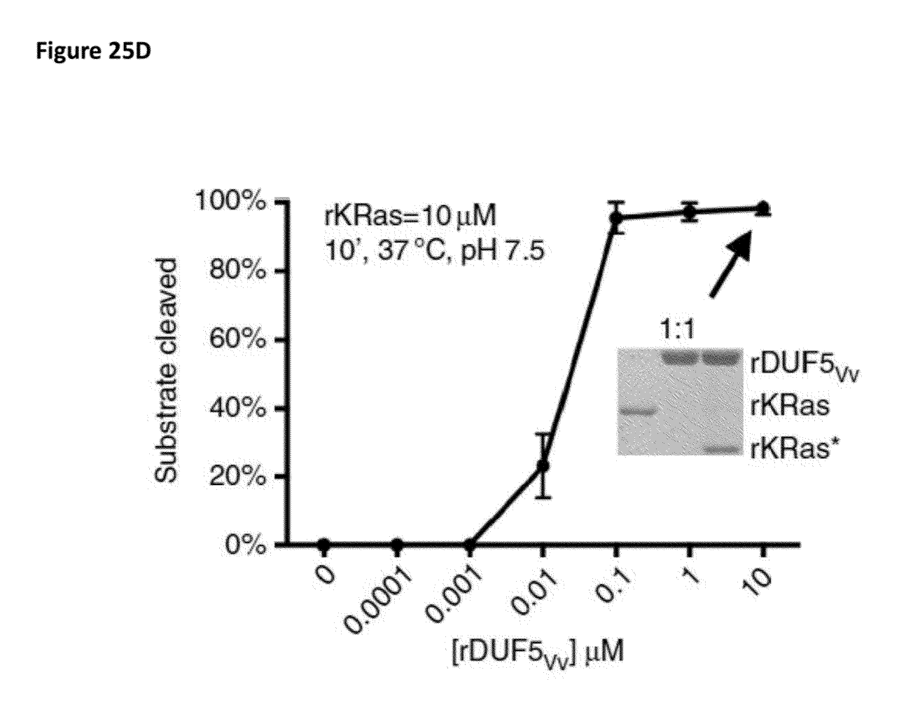

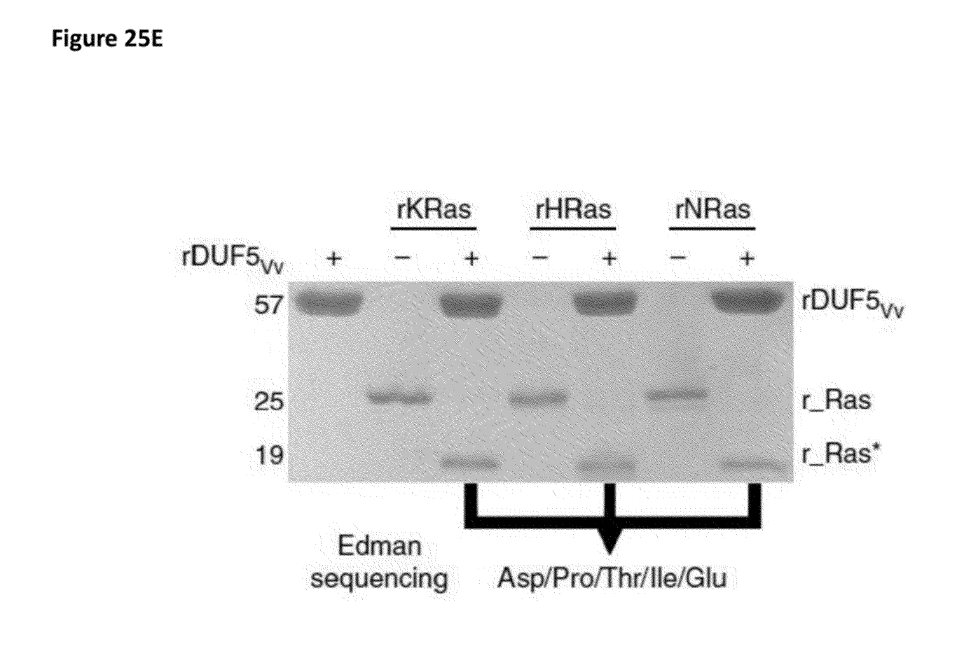

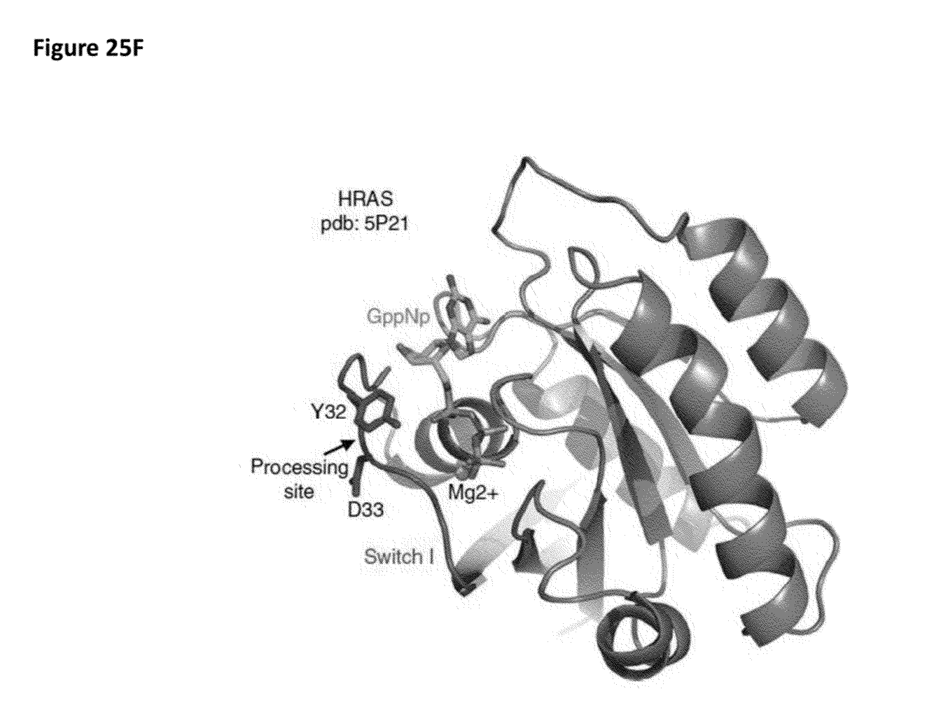

Only a few bacteria are known to specifically target Ras as a strategy to circumvent the host response and all do so by covalent attachment of nucleotide-sugar moieties to critical residues¹⁴⁻¹⁶. To investigate whether the loss of detectable Ras protein levels was due to proteolysis and/or a posttranslational modification that would mask the antibody epitope, HeLa cells were transfected to ectopically express HRas with a haemagglutinin (HA)-tag on the N terminus (HA-HRas), so as to facilitate immunoprecipitation with anti-HA antibody-coupled beads. Analysis of proteins immunoprecipitated from LFNDUF5_{Vv}+PA intoxicated cells revealed a Coomassie-stained band with a molecular weight B5 kDa smaller than the band observed in the untreated cells. Liquid chromatography-tandem mass spectrometry sequencing Q2 of tryptic peptides identified this protein as HRas, with no detection of the first three expected N-terminal peptides (FIG. 25A).

When the elution fraction was probed with anti-HA or anti-RAS10 monoclonal antibodies that detect the N terminus, a quantitative loss of the full-length protein from intoxicated cells was observed (FIG. 25B, left panel). By contrast, an isoform-specific polyclonal antibody that detects the C terminus of HRas identified two bands of HRas: one representing the full-length HA-HRas and one B5 kDa smaller. We speculate this cleaved form of HRas was present in the immunoprecipation despite lacking the HA tag, because the HA-tagged fragment remained associated with the larger C-terminal fragment in the folded protein. This experiment suggested that DUF5_{Vv} induces cleavage of Ras within the N terminus of the protein.

To verify that Ras is processed and to determine which isoforms of Ras are affected, cells were transfected to express HA-tagged KRas, NRas or HRas. In cells treated with LFNDUF5_{Vv}+PA, western blot analysis of whole-cell lysates showed that all three isoforms were cleaved at the N terminus. The anti-HA and RAS10 monoclonal antibodies directed against the N terminus did not detect KRas, NRas or HRas in treated cells, whereas isotype-specific antibodies directed against the C terminus detected the smaller processed forms (FIG. 25C). A reduction in the total protein detected by the isoform-specific antibodies was also observed. This suggests that subsequent to processing, the cleaved forms are degraded, especially for HA-NRas and HA-HRas. These data show that Ras isoforms are not modified by addition of moieties but are instead severed near the N terminus, which is a novel mechanism for Ras inactivation.

Recombinant DUF5_{Vv} can Process all Ras Isoforms In Vitro.

Two possible explanations of our results are that DUF5_{Vv} activates a previously unknown cellular peptidase or functions as a Ras peptidase itself. To distinguish whether DUF5_{Vv} directly catalyses proteolytic processing of Ras, recombinant 6×His-tagged DUF5_{Vv} (rDUF5_{Vv}) and Ras isoforms (r_Ras) were expressed in Escherichia coli and purified. When mixed together for an in-vitro reaction, rKRas was efficiently cleaved within 10 min in a concentration-dependent manner (FIG. 25D). This reaction did not require addition of any other proteins or co-factors. rHRas and rNRas were likewise efficiently processed by purified rDUF5_{Vv} (FIG. 25E).

N-terminal sequencing of KRas, HRas and NRas cleaved products revealed that all Ras isoforms were identically cleaved between Y32 and D33 (FIG. 25F). These amino acids are found within the Ras Switch I region. Processing at this site would be expected to entirely abolish Ras signalling, as Y32 is required to orient and stabilize Switch I in the active (GTP-bound) state27. Cleavage within the Switch I region would further prevent the activation of downstream signalling cascades by disrupting the Ras effector protein interactions, thereby inhibiting activation of the ERK1/2 transcriptional regulator and decreasing cell proliferation²⁸⁻³⁰.

Other DUF5 Homologues Cleave Ras.

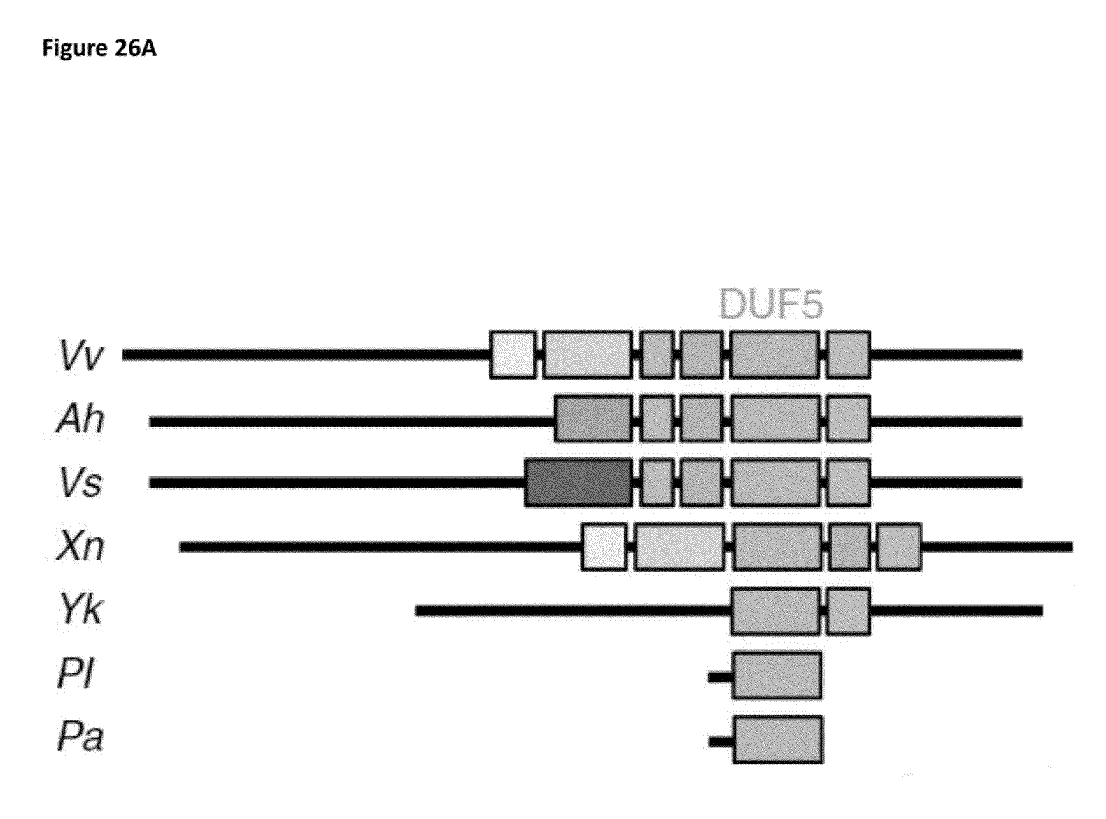

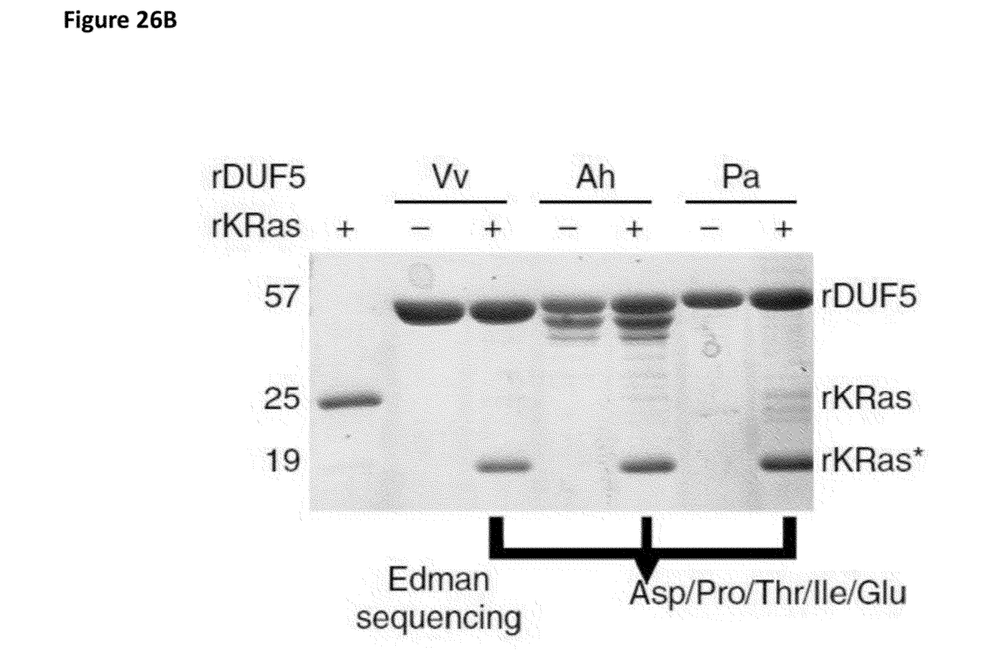

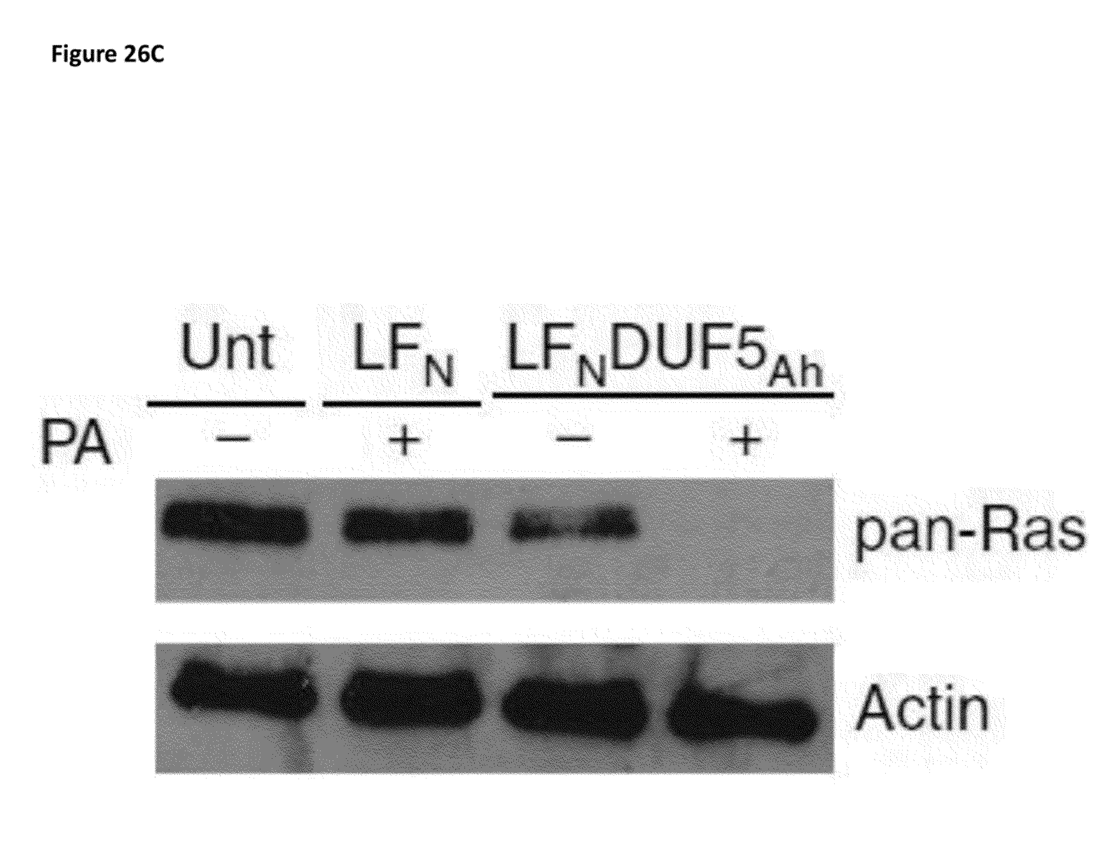

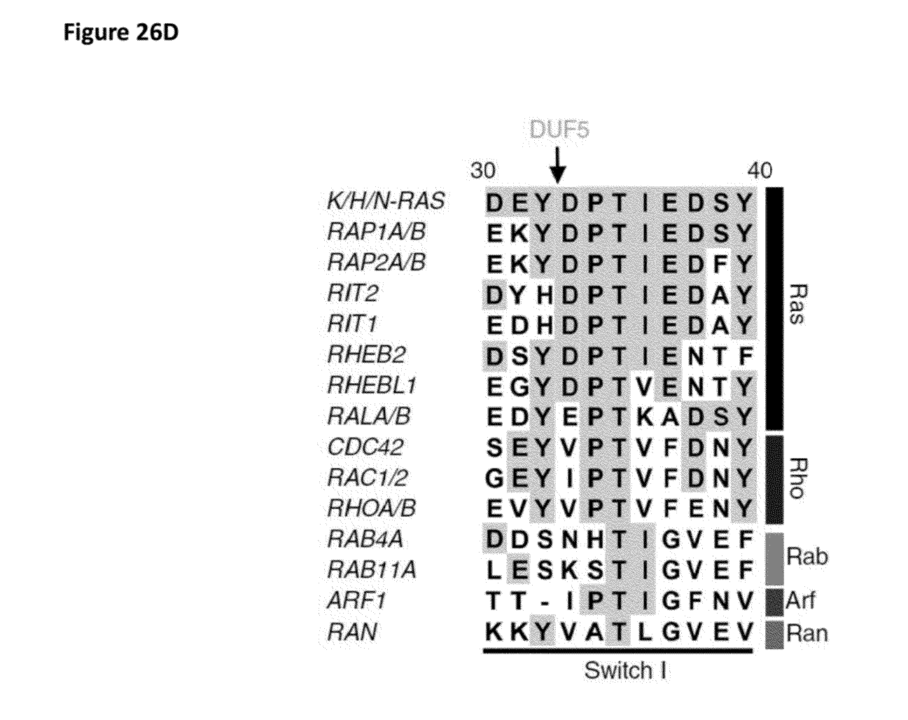

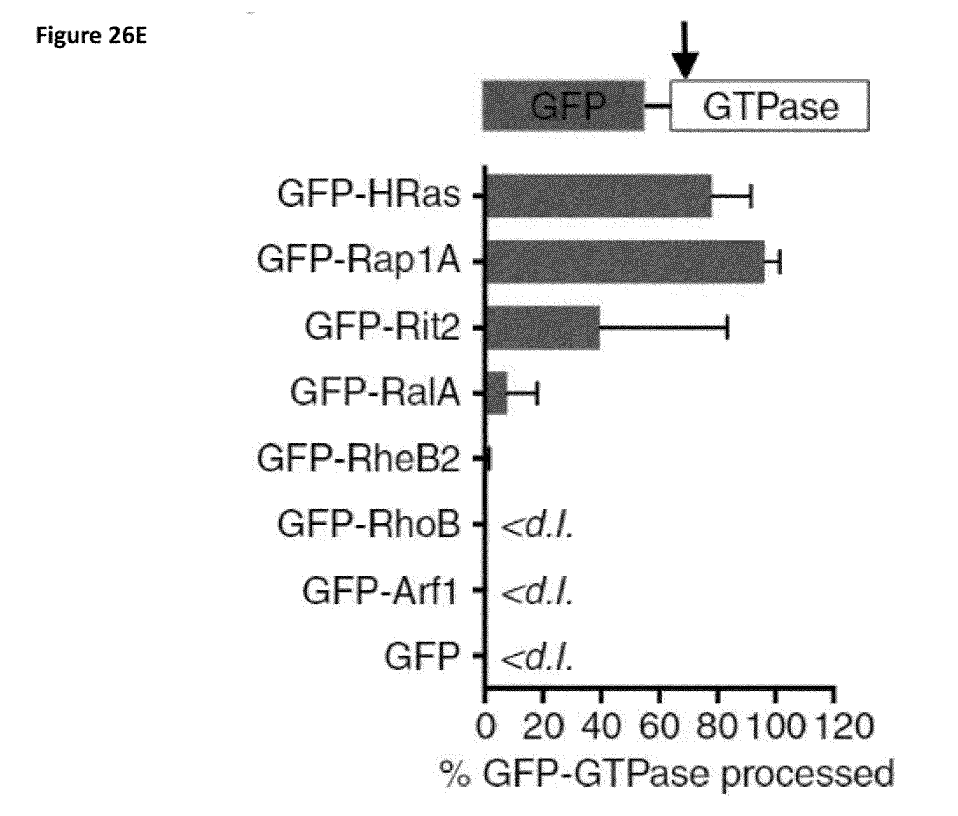

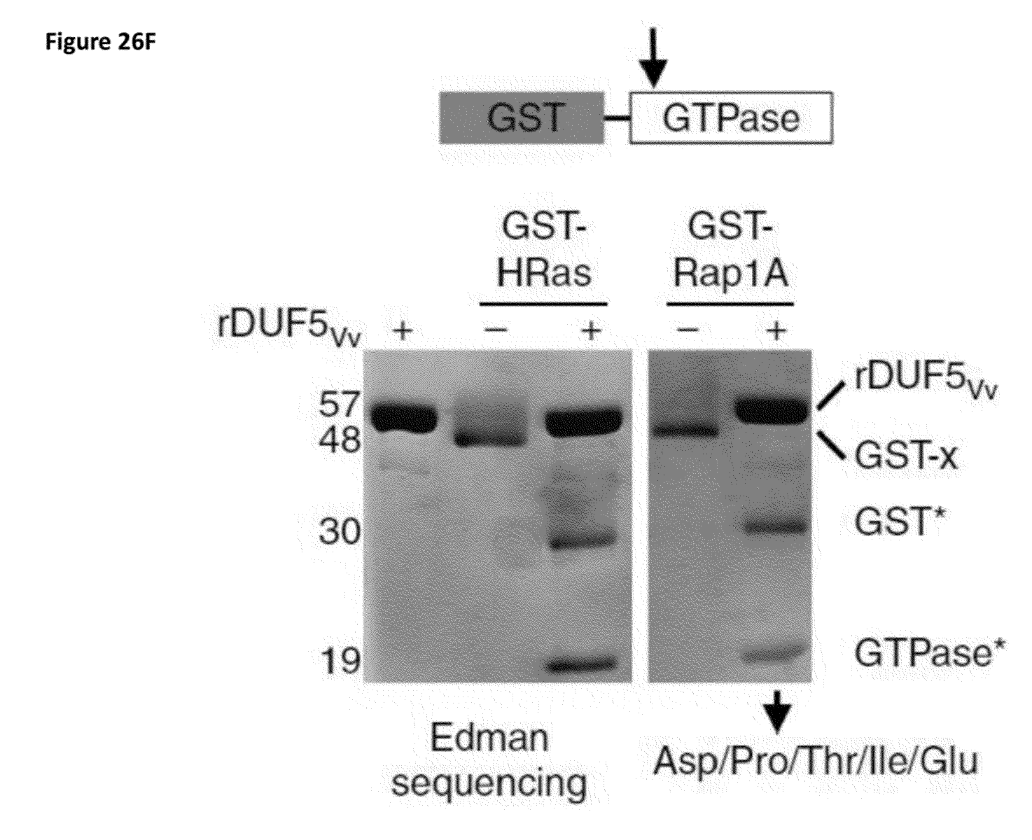

Domains similar to DUF5_{Vv} have been identified in other bacterial species (FIG. 26A). To determine whether Ras processing is a conserved function among bacteria, the effector domain from the Aeromonas hydrophila MARTX toxin (rDUF5_{Ah}) and a hypothetical effector protein from insect pathogen Photorhabdus asymbiotica (rDUF5_{Pa}) were also purified and tested for proteolytic activity. Both proteins were found to cleave rKRas in vitro with cleavage occurring between Y32 and D33 (FIG. 26B). As further validation, DUF5 was fused to LFN (LFNDUF5_{Ah}). This protein induced both cytotoxicity and Ras cleavage in intoxicated cells when delivered to cells by PA (FIG. 26C). Thus, DUF5 represents a new family of bacterial toxin effectors that catalyses site-specific processing of the Switch I region of all three major isoforms of Ras independently of any other cellular proteins.

Rap1 is Also a Substrate for DUF5_{Vv}.

Other bacterial protein toxins are known to promiscuously target a wide range of small GTPases and other cellular proteins¹⁵. As the amino acid sequence of the Switch I region of Ras is well conserved across Ras subfamily members (FIG. 26D), it was considered that DUF5_{Vv} might also cleave other small GTPases. To test this, representative Ras subfamily small GTPases fused via their N termini to enhanced GFP (EGFP) were ectopically expressed in HEK 293T cells and anti-GFP antibody was used to detect the released N-terminal fragment. In cells treated with LFNDUF5_{Vv}+PA, EGFP-HRas and EGFP-Rap1 were both cleaved with 480% efficiency. Processing of another Ras subfamily member, Rit2, was also detected in this assay, but with inconsistent efficiency, resulting in a large s.d. across multiple experiments (FIG. 26E). This indicates that Rit2 may be a low-affinity substrate resulting in experimental variation dependent on the ratio of toxin to GFP-Rit2 in each cell or sample (FIG. 26E). Other small EGFP-GTPases (RalA, RheB2, RhoB and Arf1) showed no cleavage, indicating they are not in-vivo substrates (FIG. 26E and FIG. 32).

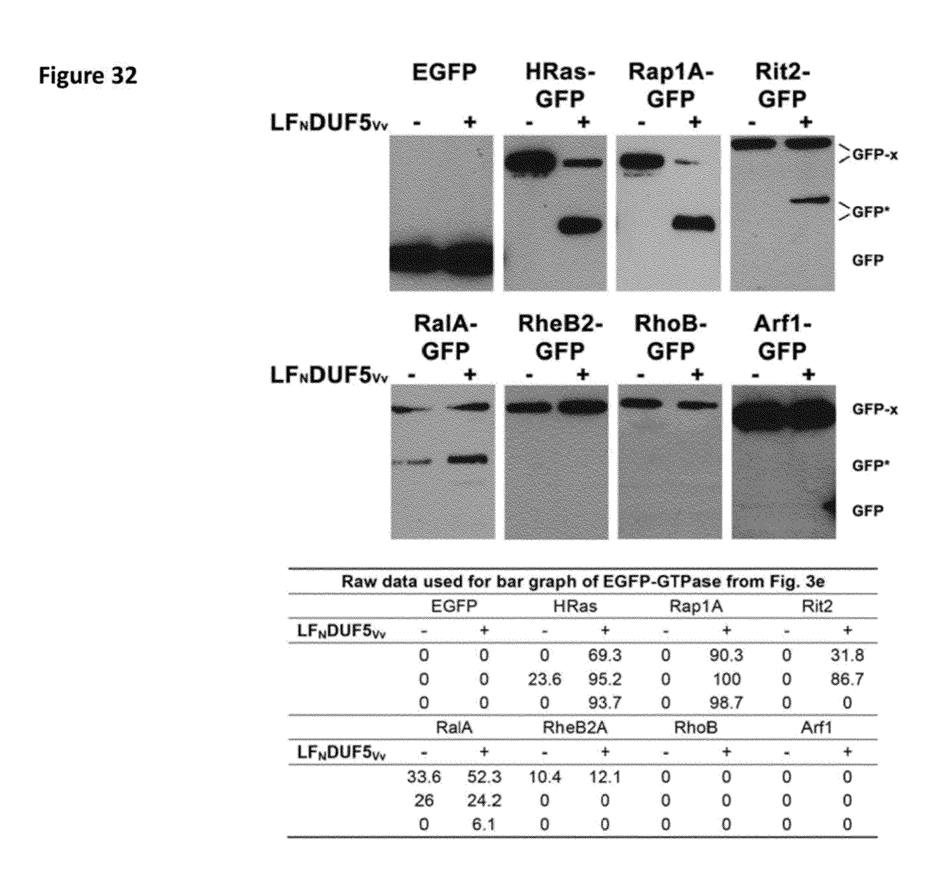

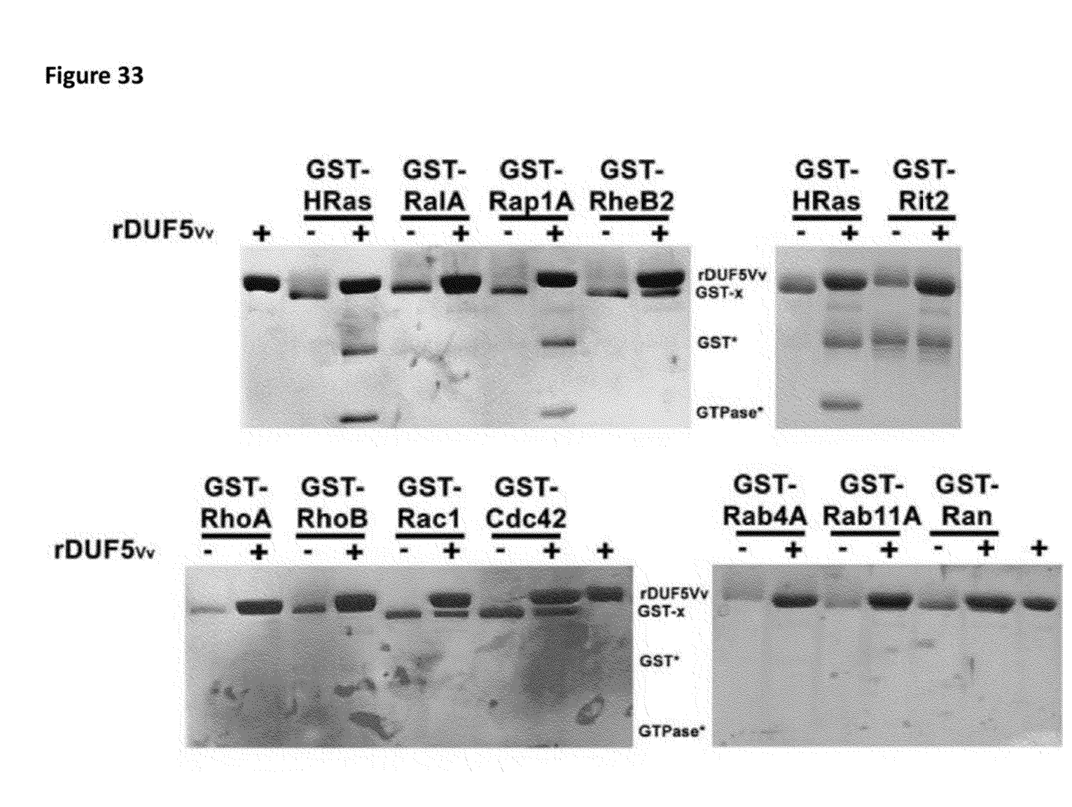

DUF5_{Vv} specificity for Ras and Rap1 was further verified biochemically. Small Ras GTPases covering the diversity of Ras subfamilies were purified as substrates for in-vitro assay to assess whether rDUF5_{Vv} could catalyse their cleavage. Among the 11 GTPases tested (FIG. 33), only Rap1 was confirmed as a DUF5_{Vv} substrate, with cleavage occurring after Y32 (FIG. 26F), whereas Rit2 was not cleaved at all, confirming that in cells this is a low-affinity substrate (FIG. 33). Other GTPases belonging to the Ras, Rho, Rab and Ran subfamilies were not processed (FIG. 33). Thus, DUF5_{Vv} is a specific protease that preferably cleaves Ras and Rap1 without cellular cofactors. The detection of Rap1 as an additional substrate is especially interesting for bacterial pathogenesis, as Rap1 activates ERK in response to bacterial components other than lipopolysaccharide and is critical for macrophage phagocytosis31,32.

DUF5_{Vv} Targets Ras During Bacterial Infection.

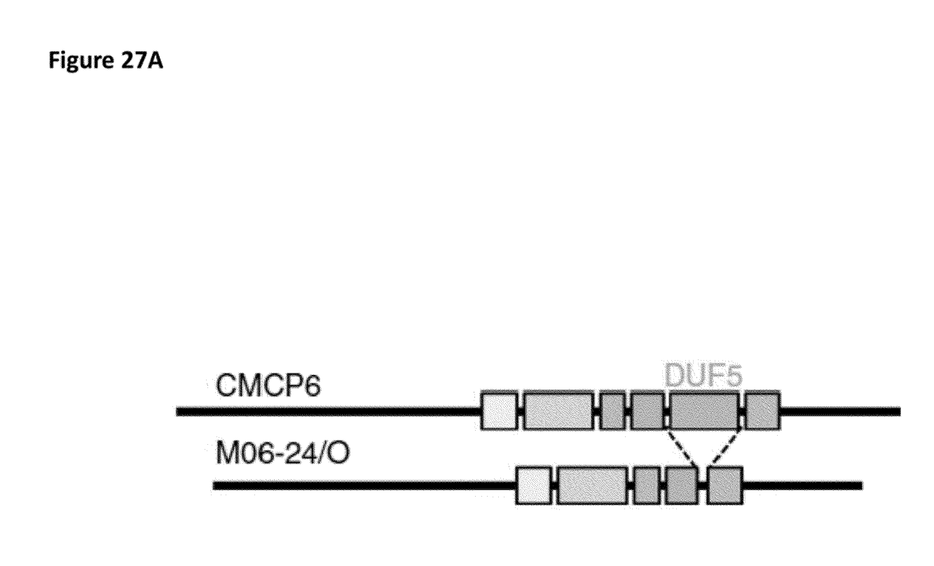

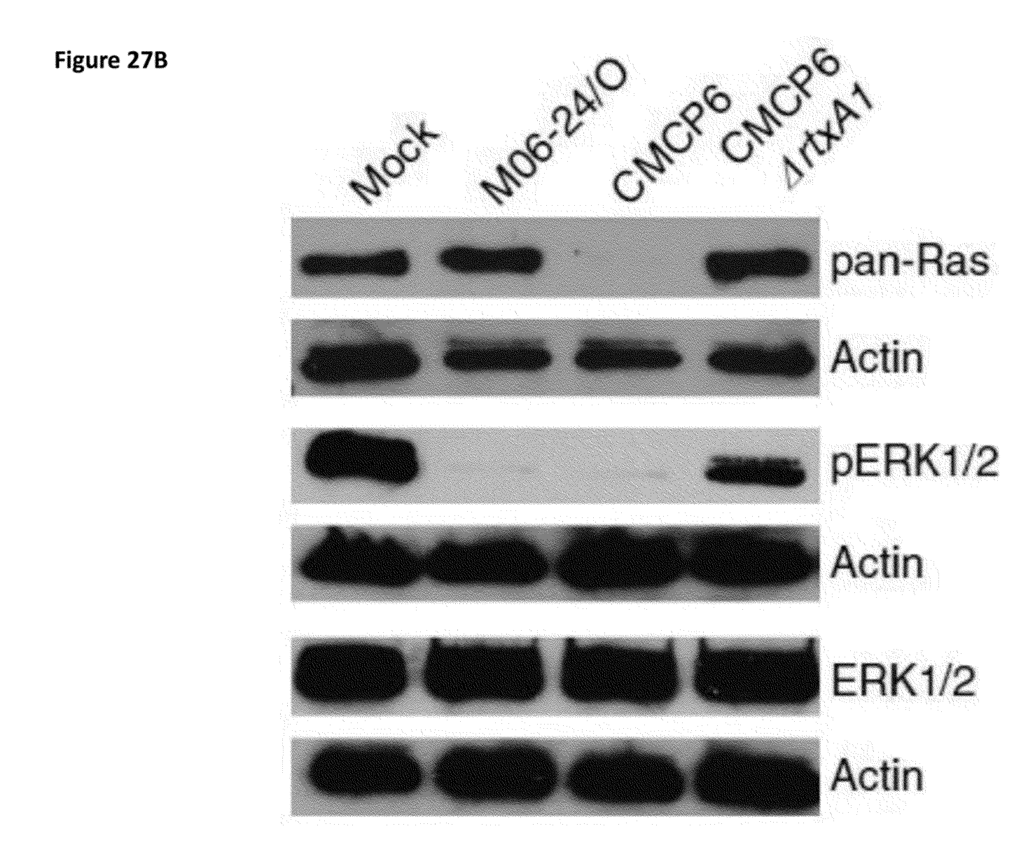

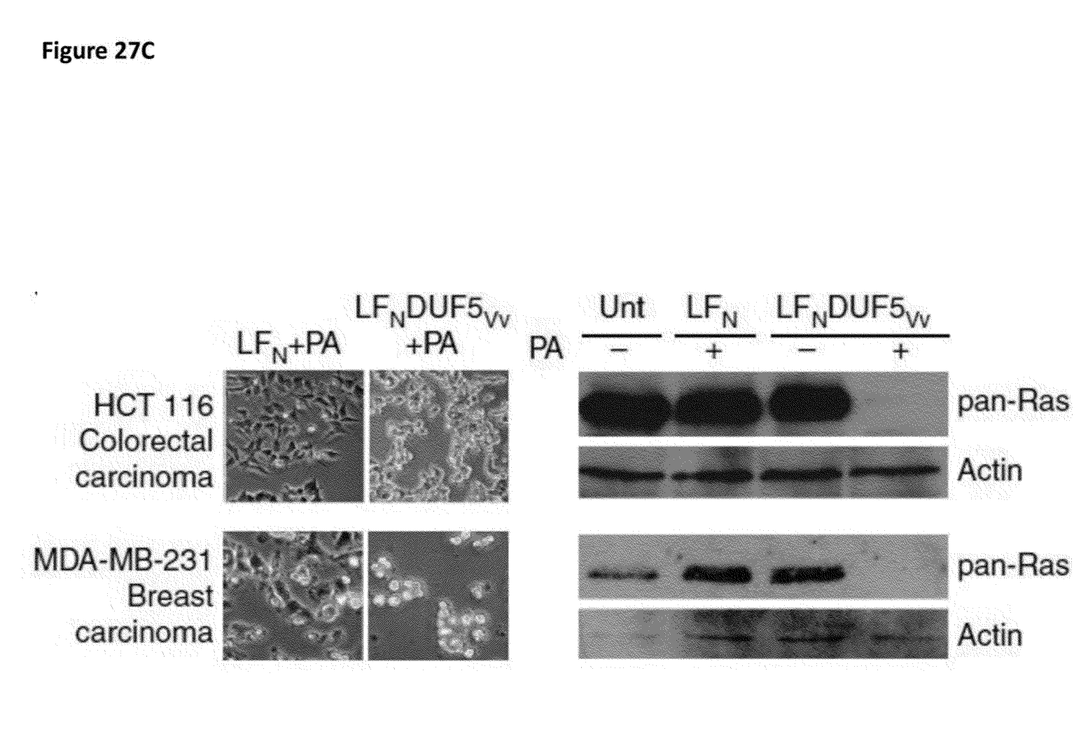

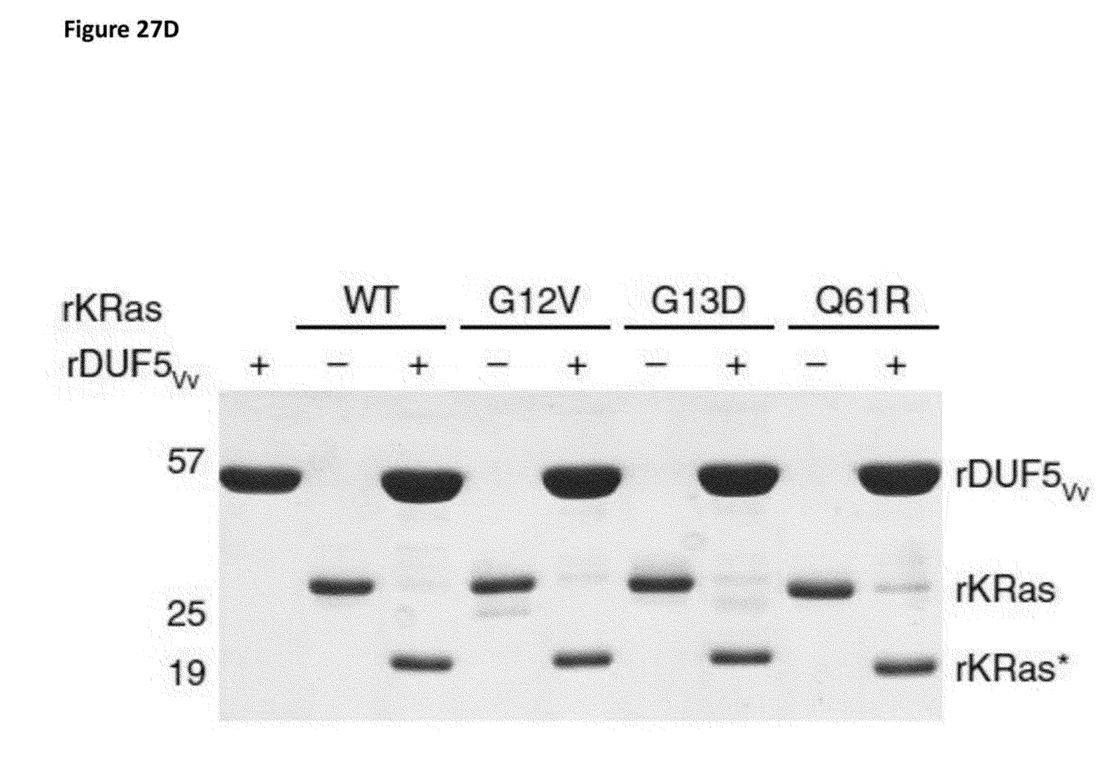

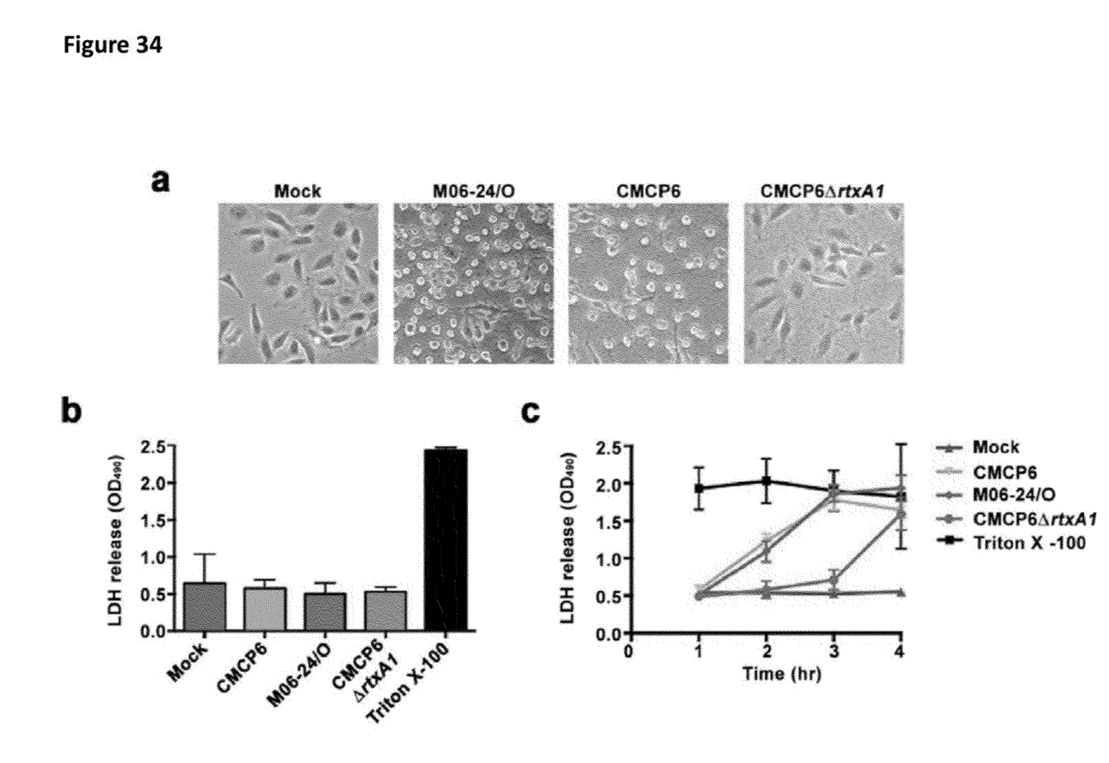

Given the importance of Ras and Rap1 in the host response to bacterial infection, it is not surprising that DUF5_{Vv} was previously shown to contribute to V. vulnificus virulence¹⁹. The strain CMCP6 produces a MARTX toxin that carries five effector domains, including DUF5_{Vv} in the fifth position. By contrast, M06-24/O produces a toxin with only four effector domains (FIG. 27A), having undergone a genetic recombination that resulted in an in-frame deletion of the DNA sequence for the DUF5_{Vv} domain^{19,33}. As a result of the loss of DUF5_{Vv}, M06-24/O is tenfold less virulent than CMCP6 (ref. 19). The increased virulence of CMCP6 was found to be specifically due to DUF5_{Vv} 19, even though both toxin forms induce cellular necrosis^{34,35} (FIG. 34).

To link this defect in virulence to Ras activation and demonstrate that Ras can be processed during normal toxin delivery, HeLa cells were co-cultured for 1 h with V. vulnificus and proteins in cell lysates were analysed by western blotting. Cells treated with wild-type bacteria producing full-length active MARTX toxin no longer showed detectable Ras or pERK1/2. This inactivation was dependent on an intact rtxA1 toxin gene, as a null mutation in rtxA1 of V. vulnificus CMCP6 did not show loss of detectable Ras or pERK1/2. Further, co-culture of cells with V. vulnificus M06-24/O, which produces the MARTX toxin naturally missing DUF5_{Vv}, did not affect Ras, linking this MARTX-dependent activity specifically to the DUF5_{Vv} effector domain. Interestingly, cells treated with M06-24/O unexpectedly still showed a reduction of pERK1/2, revealing that these multifunctional toxins probably have redundant strategies to inactivate ERK during infection (FIG. 27B).

Oncogenic KRas is Processed by DUF5_{Vv}.

Point mutations resulting in constitutive activation of Ras have long been associated with many different types of adenocarcinomas⁵⁻⁷. The discovery of a novel bacterial toxin mechanism to halt cell proliferation through processing of Ras is not only important for understanding the function of bacterial toxins during infection but also presents an opportunity to potentially target Ras during carcinogenesis through delivery of DUF5. This strategy would be most successful if mutant forms of Ras found in cancer cells are also DUF5 substrates.

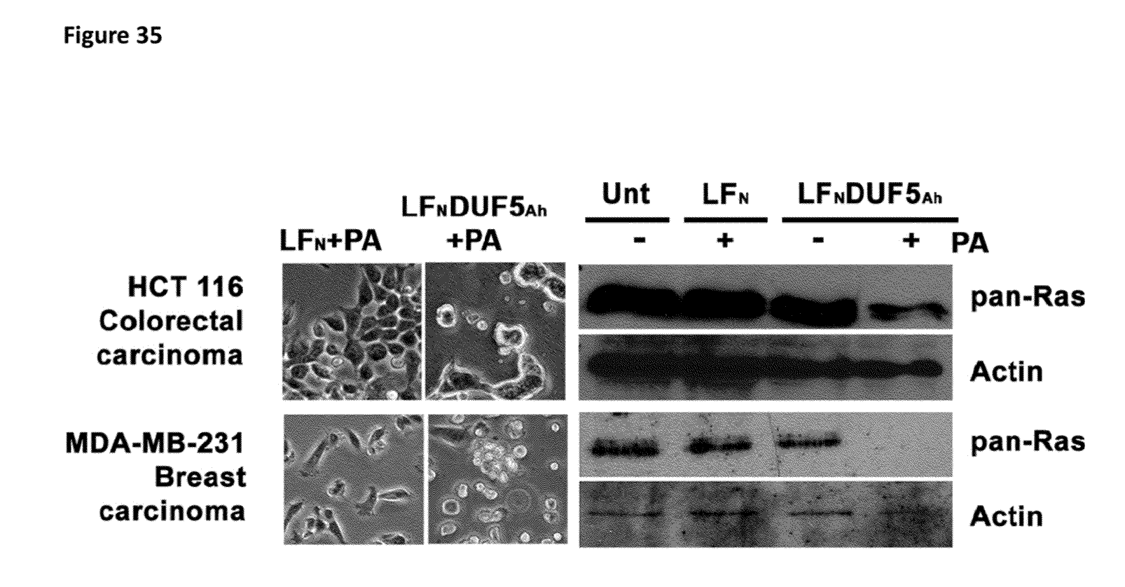

When HCT116 colorectal carcinoma cells, which express KRas with a G13D mutation, were intoxicated with PA in combination with LFNDUF5_{Vv} (FIG. 27C) or LFNDUF5 (FIG. 35), significant cell morphological changes were observed and Ras was undetectable by western blotting. Similar results were obtained with the breast cancer cell line MDA-MB-231 that likewise carries the KRas G13D mutation. This cell line also contains a G464V mutation in B-Raf36, an effector of both Ras and Rap1 (ref 37), demonstrating that DUF5_{Vv} can effectively intoxicate cells even if they have additional activating mutations downstream of Ras and Rap1.

As further demonstration that DUF5_{Vv} could be employed as a cancer treatment, rKRas was modified to carry three of the most common Ras mutations associated with tumorigenesis: G12V, G13D or Q61R7. All three mutant forms of KRas were confirmed as in vitro substrates for rDUF5_{Vv}-dependent site-specific processing (FIG. 27D). Thus, the ability of DUF5_{Vv} to cleave KRas is unaffected by the most common RAS mutations. Overall, these data show that cells carrying constitutively active forms of Ras are not protected from DUF5_{Vv} cytotoxicity and thus DUF5_{Vv} is a valid candidate for use as an anti-tumour agent.

Discussion

MARTX toxins are large bacterial toxins that carry multiple effector domains, each with a specific enzymatic activity. DUF5_{Vv}, the extra effector domain of the MARTX toxin from the most virulent strains of the sepsis-causing pathogen V. vulnificus, was previously shown to be highly cytotoxic for mammalian cells, although the mechanism of this cytotoxicity was unknown²⁰. In this work, we demonstrate that DUF5_{Vv} is a representative member of a new family of bacterial toxin effectors that catalyse site-specific processing of the Switch I region of Ras and Rap1. Activated Ras or Rap1 would normally interact with downstream effectors such as c-Raf, to stimulate the phosphorylation of ERK1/2. In particular, Y32 in the Switch I region plays an important role in stabilizing the GTP-bound form of Ras and its interaction with the Raf kinases²⁷. Thus, it is predicted that DUF5_{Vv} cleavage between Y32 and D33 would destabilize the Switch I and presumably the interactions of Ras and Rap1 with their binding partners. As Ras and Rap1 form parallel pathways that relay signals from surface receptors and guanine nucleotide exchange factors to activate ERK1/2, disabling both small GTPases simultaneously nullifies all downstream signaling pathways³⁸, resulting in the complete loss of pERK1/2 in DUF5_{Vv}-treated cells. In the context of bacterial infection, this is important to inactivate innate immune responses, accounting for the direct linkage of this toxin effector domain to virulence of V. vulnificus. We propose that the DUF5 effector domain be renamed RRSP for Ras/Rap1-specific protease, acknowledging its site-specific processing of the Switch I region of Ras and Rap1.

As small GTPases are responsible for regulating essential cell functions, many other bacterial protein toxins and effectors target GTPases by posttranslational modification or by manipulating Q3 their function¹⁵. However, few of these toxins target Ras specifically, for example, Pseudomonas aeruginosa ExoS ADP ribosylates R41 of Ras and Rap³⁹⁻⁴¹, and thereby directly inhibits phagocytosis in mice⁴². However, ExoS also has broad substrate recognition including other GTPases⁴³ and other proteins such as moesin and vimentin^{16,44,45.} Similarly, Clostridium sordellii lethal toxin TcsL (also known as LT) has been shown to glucosylate Ras at T35 in the Switch I^{46,47} resulting is cellular apoptosis⁴⁸. In addition, TcsL UDP-glucosylates other small Ras, Rap, Ral, Rho and Rac GTPases with some specificity differences depending on strain⁴⁹. Through a similar process, Clostridium perfringens large toxin TpeL modifies T35 of Ras and, to a lesser extent, Rap1 and possibly Rac1, except it preferentially uses UDP-Nacetylglucosamine as a sugar donor^{50,51}.

The unique feature of RRSP demonstrated here is its irreversible mechanism of action by cleaving rather than modifying Ras and Rap1. The biochemical basis for the specificity of RRSP for Ras and Rap1 should be explored further in the future. Although it is possible that the specificity is dictated by the conservation of the amino acid sequence in the Ras and Rap1 Switch I regions, it is more likely to be that recognition of the target is multifactorial depending on a multifaceted protein—protein interaction between RRSP and Ras or Rap1. This possibility is supported by studies of Clostridium difficile toxin TcdB recognition of RhoA as a substrate for glucosylation, which is mediated in part by specificity for target residue T37 in the Switch I region⁵², but also by Ser73 outside the Switch I⁵³. In addition, amino acids of TcdB essential to discriminate substrate are found outside the catalytic site, further indicating that specificity of TcdB from Rho in not driven solely by the Switch I sequence⁵⁴.

In addition to protein—protein interactions, specificity of RRSP for Ras and Rap1 may include spatial localization to anionic membranes or specificity for the active or inactive state conformation when bound to GTP or GDP, respectively. However, in cells, we routinely observed 100% processing of all Ras isoforms in as little as 30 min and we also observed 100% cleavage of KRas G12V, G13D and Q61R in vitro, despite not controlling the GTP or GDP state using buffers. These data would seem to support the hypothesis that RRSP can target both active and inactive forms of Ras and thereby access both membrane and cytoplasmic pools of Ras. In addition, as the Switch I region undergoes structural changes with activation state, and both active and inactive forms of Ras seem to be substrates for RRSP, we suppose specificity is at least in part driven by protein—protein interaction outside the Switch I region and this will be explored in the future through detailed structural and binding studies.

A critical question for bacterial infection is how the processing of Ras and Rap1 contributes to increased virulence. The MARTX toxin of V. vulnificus is known to play a role during infection both in paralysing phagocytic cells⁵⁵ and in breaching the epithelial barrier to promote spread of the bacterium from the intestine to other organs⁵⁶⁻⁵⁸. Overall, small GTPases play a central role in the barrier function of epithelial layers such that loss of this control could contribute to bacterial spread across the intestinal barrier¹⁵. In particular, Ras and Rap1 are essential for sensing and signalling pathogen-associated molecular patterns and for regulating inflammatory responses of the host organism^{15,59}. Ras and Rap1 function in response to bacterial components such as LPS and for macrophage phagocytosis, activating the ERK1/2 Q4 pathway cascade^{31,32}; in the context of bacterial infection, inhibition of these cascades would slow down the host response to bacterial infection, such that V. vulnificus strains that carry this domain are more virulent¹⁹.

A final impact of our discovery is the possibility that the RRSP effector domain could be deployed across the cell membrane to specifically target tumour cells using different delivery strategies. More than three decades after the discovery of Ras implication in cancer development, targeting Ras remains one of the hardest challenges of cancer research and drug discovery. Here, we propose that proteins in this new RRSP effector family could be employed immediately as research tools, but in the future developed as new anti-cancer therapeutic agents. Of particular immediate interest, re-engineered PA selectively targeting cancer cells could be used to deliver LFNDUF5 into cells to destroy Ras and thereby deregulate tumour growth and proliferation. This approach has already been validated in cell systems in which PA was fused to the epidermal growth factor for delivery of LFN-tethered cargo into cancer cells with upregulated expression of the epidermal growth factor receptor⁶⁰. This system has also been proven with PA modified to bind to the HER2 receptor, a protein strongly upregulated in tumour cells, in particular breast cancers⁶¹. As alternative future approaches, RRSP effector domains could be fused to specific antibodies for use as an immunotoxin⁶², or expressed and delivered by Salmonella bacteria that home to solid tumours⁶³. It could also be expressed by viruses engineered to specifically infect cancer cells⁶⁴. The ability of RRSP to cleave both normal and mutant forms of Ras indicates that any developed reagent could be successful whether used for Ras cancers, non-Ras cancers, or other Ras-associated diseases.

Methods

General Molecular Biology Techniques.

E. coli DH5a and TOP10 cells (Life Technologies) were grown at 37° C. in Luria-Bertani liquid or on agar medium supplemented with either 100 μg ml⁻1 ampicillin or 50 μg ml⁻1 kanamycin, as needed. Common reagents were obtained from Sigma-Aldrich, Fisher or VWR, and common restriction enzymes and polymerases were obtained from New England Biolabs or Life Technologies. Custom DNA oligonucleotides were purchased from Integrated DNA Technologies (Coralville, Iowa). Plasmids were prepared by alkaline lysis followed by precipitation in ethanol or purified using Epoch spin columns according to the manufacturer's recommended protocol. A Qiagen Midi Prep kit was used for preparation of plasmids used in yeast transformations. Plasmids were introduced into E. coli by electroporation and into HeLa cells by transfection using polyethylenimine (PEI).

Yeast Non-Essential Gene Deletion Screen.

The Life Technologies YKO yeast deletion library covering all non-essential genes was replicated from stocks at the Northwestern University High Throughput Analysis Laboratory using a Genetix QPixII Automatic colony picker. Each strain from the library was subsequently grown in 1 ml yeast extract peptone dextrose with addition of 50 μg ml⁻1 G418. After overnight growth at 30° C. with agitation, each strain was transformed with plasmid pYC-C2 using a PLATE solution method and transformants were selected on synthetic complete agar without uracil and with 2% glucose to repress DUF5_{Vv}-C2 expression. Colonies were patched with toothpicks onto synthetic complete agar supplemented with 2% galactose and 1% raffinose to induce DUF5_{Vv}-C2 expression. Initial positive selection was defined as yeast that formed a patch when grown on galactose. These were subsequently rescreened in a dilution plating assay as previously described²⁰ and those with a plating efficiency comparable to a strain transformed with empty vector were considered validated hits. Identified strains were analysed and classified based on information in the Saccharomyces Genome Database (www.yeastgenome.org), last accessed on 25 Oct. 2014.

Intoxication of Cells with Proteins Fused to LFN.

HeLa, HCT116 and HEK293 cells were grown at 37° C. with 5% CO₂ in DMEM medium (Life Technologies) with 10% fetal bovine serum (Gemini Bio-Products, West Sacramento, Calif.), 100 Uml1 penicillin and 1 μg ml⁻1 streptomycin. Purification of LFN, LFNDUF5_{Vv} and LFNDUF5₁ₕ has been previously described20. PA purified as previously described⁶⁵ was provided by Shivani Agarwal (Northwestern University). Cell lines were seeded overnight into tissue culture-treated dishes and flasks, except for HCT116 cells, which were seeded for 48 h. Before intoxication, the media was exchanged for fresh media and then 7 nM PA and 3 nM LFN-tagged toxins were added to the media and incubated for the times indicated in the legend at 37° C. with 5% CO₂. Cells were imaged at ×10 at times indicated in the legend using a Nikon TS Eclipse 100 microscope equipped with a Nikon CoolPix 995 digital camera or processed for western blotting or colony formation as detailed below.

Western Blotting.

A total of 2.5-5×10⁴ treated cells were washed with PBS, then resuspended in 120 ml of 2× Laemmli sample buffer and boiled for 10 min. Ten microlitres of lysate were separated by SDS-PAGE and transferred to nitrocellulose (Amersham) using the Bio Rad Trans-Blot Turbo system. Nitrocellulose membranes were blocked overnight at 4° C. in 5% (w/v) powdered milk diluted in Trisbuffered saline containing 0.001% Tween-20 (TBS-T). Immunodetection of proteins was conducted as previously described²⁰, using primary antibodies purchased from Cell Signaling Technologies (p44/42 MAPK (ERK1/2) rabbit mAb 137F5 (1:1,000), phospho-p44/42 (ERK1/2) rabbit mAb 197G2 (1:1,000), p38 MAPK rabbit polyclonal 9212 (1:1,000) and phospho-p38 rabbit mAb 12F8 (1:1,000)), EMD Millipore (pan-Ras mouse mAb RAS10 (05-516, 1:1,000)), Thermo Scientific (HRas PA5-22392 (1:1,000), KRas PA5-27234 (1:1,000) and NRas PA5-28861 (1:1,000)) and Sigma-Aldrich (H6908 rabbit polyclonal (1:5,000), actin mouse mAb AC-40 (1:1,000) and Tubulin T6074, (1:10,000)). Antibody binding to proteins was detected using anti-mouse (1:5,000) or anti-rabbit (1:5,000) secondary antibodies conjugated to horseradish peroxidase from Jackson Immuno Research and developed using SuperSignal WestPico chemiluminescent reagents (Thermo Scientific) and X-ray film. For serial detection of proteins and detection of the actin-loading controls from the same nitrocellulose membrane, membranes were washed in TBS-T for 10 min and then stripped of antibody by washing the membrane for 10 min with stripping buffer (1.5% glycine, 1% Tween-20, 0.1% SDS). After two more 10-min washes with TBS-T, the membrane was re-probed for other proteins. Tubulin-loading controls were performed by cutting the membrane horizontally to separate the upper loading control portion containing tubulin from the lower portion containing the small Ras family GTPases. Uncropped western blottings are not shown but are provided herein but are provided in the Supplementary Material for Antic et al., Nat. Commun. 2015 Jun. 8; 6:7396, which is incorporated herein by reference in its entirety.

Ras G-LISA.

Active (GTP-bound) Ras in intoxicated cells was measured using the Ras G-LISA activation colorimetric assay kit from Cytoskeleton, Inc. (Denver, Colo.). HeLa cells were seeded into 10-cm² tissue culture-treated dishes and grown to ˜80% confluency, at which time the cells were intoxicated with LFN proteins in combination with PA for 24 h as described above. Cells were collected in the lysis buffer and total protein content was determined by the Precision Red assay using reagents supplied with the kit. The lysate was frozen in a dry ice-ethanol bath and stored at 80° C. Active Ras in each lysate was then determined according the manufacturer's protocol. This kit used the pan-Ras RAS10 mAb for detection of active Ras and this antibody was subsequently obtained directly from Millipore for western blotting detection of Ras as described above.

Clonogenic Colony-Formation Assay.

A total of 10⁵ HeLa cells were seeded into six-well dishes overnight, intoxicated with LFN protein as described above and assessed by a clonogenic colony-formation assay as described previously⁶⁶. Briefly, cells were released from wells with 0.25% trypsin/EDTA (Sigma), counted in a hemocytometer and then diluted. The number of cells indicated was replated in fresh media in duplicate. After 14 days, cells were fixed with 70% ethanol and stained with 0.5% crystal violet, and colonies of more than 50 cells were counted. The surviving fraction was compared with cells treated with LF_{N}+PA.

Ectopic Expression of HA-Tagged Ras Isoforms.

Plasmids for ectopic expression of HA-HRas (pcDNA3-HA-HRas_wt, 14723) and HA-NRas (pCGN NRas wt, 39503) were obtained from Addgene (Cambridge, Mass.). Plasmids for overexpression of HA-KRas and HA-KRas G12V were obtained from Athanasios Vassilopoulos (Northwestern University). Plasmid DNA (2 mg) was mixed with 90 ml PEI diluted in incomplete DMEM media, vortexed 15 times and then incubated for 15 min at room temperature. Seven hundred microlitres of complete DMEM were added into the plasmid-PEI mix and the whole volume was added to HeLa cells. After 24 h, cells were intoxicated as described above.

Immunoprecipitation of HA-HRas and Mass Spectrometry.

HeLa cells, either untreated or intoxicated with LFNDUF5_{Vv}+PA as described above, were washed with cold PBS and then resuspended in RIPA buffer (50 mM Tris-HCl pH 7.5, 150 mM NaCl, 0.1% SDS, 0.5% sodium deoxycholate, 1% Triton and ‘cOmplete’ protease inhibitors). HeLa cell lysates were incubated with 50 ml of anti-HA agarose beads (Sigma) for 2 h at 4° C. under mild agitation. Beads were then washed five times with 500 ml of RIPA buffer and five times with 500 ml of washing buffer (50 mM Tris-HCl pH 7.5, 150 mM NaCl). Proteins bound to the beads were eluted with 3M sodium thiocyanate buffer (50 mM Tris-HCl pH 7.5, 150 mM NaCl). Elution fractions were analysed by SDS-PAGE followed by Coomassie staining or immunoblotting using anti-HA and isotype-specific anti-HRas antibody as described above. The smaller HRas band was excised from the gel, put in water and then frozen for shipping. Trypsin digestion followed by liquid chromatography-tandem mass spectrometry on the Thermo LTQ-FT Ultra spectrophotometer was conducted at the University of Illinois at Chicago Mass Spectrometry, Metabolomics and Proteomics Facility according to their standard protocols.

Preparation of 6×His- or GST-Tagged Small GTPases.

DNA sequences corresponding to KRas (KRas4B, NP_{—}004976.2), HRas (NP_{—}001123914.1) and Q5 NRas (NP_{—}002515.1) genes were amplified from templates as described above, using primers designed for ligation-independent cloning, and the products were cloned into the pMCSG7 expression vector by ligation-independent cloning⁶⁷. The G12V, G13D and Q61R mutations were introduced by site-directed mutagenesis using the pMCSG7-KRas vector as a template. Primers are listed in the Table 2 below:

TABLE 2
Oligonucleotides Used in this Example

| | |
|---|---|
| DUF5 VV FWD | TACTTCCAATCCAATGCTCAAGAGCTGAAAGAA |
| | AGAGCAAAAG |
| | (SEQ ID NO: 37) |
| DUF5 VV REV | TTATCCACTTCCAATGCTACAAACTGCCCTTGA |
| | ACGTG |
| | (SEQ ID NO: 38) |
| DUF5 AH FWD | TACTTCCAATCCAATGCTCCGGGCAAAACGGTG |
| | GTGACG |
| | (SEQ ID NO: 39) |
| DUF5 AH REV | TTATCCACTTCCAATGCTAGACATCGGCGTACT |
| | CGACCCGC |
| | (SEQ ID NO: 40) |
| DUF5 PA FWD | TACTTCCAATCCAATGCTCCATTACTCCATGAC |
| | CTCATCACC |
| | (SEQ ID NO: 41) |
| DUF5 PA REV | TTATCCACTTCCAATGCTACACATCATCATAAC |
| | ACTTGCG |
| | (SEQ ID NO: 42) |
| KRAS FWD | TACTTCCAATCCAATGCTATGACTGAATATAAA |
| | CTTGTGGTAGTTGGAGCTGG |
| | (SEQ ID NO: 43) |
| KRAS REV | TTATCCACTTCCAATGCTACATAATTACACACT |
| | TTGTCTTTGACTTCTTTTTCTTC |
| | (SEQ ID NO: 44) |
| HRAS FWD | TACTTCCAATCCAATGCTATGACGGAATATAAG |
| | CTGGTGGTGGTG |
| | (SEQ ID NO: 45) |
| HRAS REV | TTATCCACTTCCAATGCTAGGAGAGCACACACT |
| | TGCAGCTC |
| | (SEQ ID NO: 46) |
| NRAS FWD | TACTTCCAATCCAATGCTATGACTGAGTACAAA |
| | CTGGTGGTGG |
| | (SEQ ID NO: 47) |
| NRAS REV | TTATCCACTTCCAATGCTACATCACCACACATG |
| | GCAATCCC |
| | (SEQ ID NO: 48) |
| EGFPC3-GST FWD | GCTTCGAATTCTGCACCCGGGTGGTCTGGTTCC |
| | GCGTGGA |
| | (SEQ ID NO: 49) |
| EGFPC3-GST REV | CTAGATCCGGTGGATCCCCTCAGTGGTGGTGGT |
| | GGTGGTGC |
| | (SEQ ID NO: 50) |
| KRAS_G13D FWD | TAGTTGGAGCTGGTGACGTAGGCAAGAGTGC |
| | (SEQ ID NO: 51) |
| KRAS_G13D REV | GCACTCTTGCCTACGTCACCAGCTCCAACTA |
| | (SEQ ID NO: 52) |
| KRAS_Q61R FWD | GATATTCTCGACACAGCAGGTAGAGAGGAGTAC |
| | AGTGCAATG |
| | (SEQ ID NO: 53) |
| KRAS_Q61R REV | CATTGCACTGTACTCCTCTCTACCTGCTGTGTC |
| | GAGAATATC |
| | (SEQ ID NO: 54) |

Plasmids were confirmed to be accurate by DNA sequencing and then transformed into E. coli BL21(DE3). Cultures of E. coli were grown at 25° C. in Terrific Broth supplemented with 100 μg ml⁻1 ampicillin to an OD₆₀₀ of 0.6-0.7 and then induced with 1 mM isopropyl-β-D-thiogalactoside and growth was continued at 18° C. for ˜18 h. Bacteria were harvested by centrifugation, re-suspended in buffer A1 (50 mM Tris pH 7.5, 500 mM NaCl, 10 mM MgCl2, 0.1% Triton X-100, 5 mM β-mercaptoethanol) and lysed by sonication. After centrifugation at 30,000 g for 30 min, the soluble lysate was loaded onto a 5-ml HisTrap column using the ÄKTA protein purification system (GE Healthcare). The column was washed with buffer B1 (10 mM Tris pH 7.5, 500 mM NaCl, 10 mM MgCl2, 50 mM imidazole) followed by elution in the same buffer with 500 mM imidazole (buffer C1). Proteins were further purified by size-exclusion chromatography (Superdex 200 (26/60), GE Healthcare) in buffer D1 (10 mM Tris-HCl pH 7.5, 500 mM NaCl, 10 mM MgCl2, 5 mMb-mercaptoethanol). GST-fusion GTPases were obtained from Seema Mattoo (Purdue University, IN), and expressed and purified as previously reported68.

Preparation of 6×His-Tagged DUF5 Proteins.

DNA sequences corresponding to DUF5_{Vv} (V. vulnificus CMCP6-MARTX_{Vv} Q3596-L4089, NP_{—}759056.1), DUF5 (A. hydrophila ATCC7966-MARTXAh P3069-V3570-locus WP_{—}011705266) and DUF5ₚₐ (P. asymbiotica ATCC43949-P41-V532 locus WP_{—}011705266) were amplified from their respective genomes using primers designed for ligation-independent cloning and the products were cloned into the pMCSG7 expression vector by ligation-independent cloning⁶⁷. Primers are listed in Supplementary Table 1. Plasmids were confirmed to be accurate by DNA sequencing and then transformed into E. coli BL21(DE3). Cultures were grown in Terrific Broth supplemented with 100 μg ml⁻¹ ampicillin at 37° C. until OD₆₀₀=0.7-0.8 and then induced with 1 mM isopropyl-β-_{D}-thiogalactoside at 18° C. for ˜18 h. Proteins were purified as described above for Ras proteins, except all buffers were adjusted to pH 8.3 instead of 7.5.

In-Vitro Cleavage Assay and N-Terminal Sequencing.

rKRas, rHRas, rNRas and GST-fused small GTPases were incubated with rDUF5 proteins at equimolar concentrations (10 mM) in 10 mM Tris pH 7.5, 500 mM NaCl, 10 mM MgCl2 at 37° C. for 10 min, unless otherwise indicated. Reactions were stopped by adding 6× Laemmli sample buffer and incubating the sample at 90° C. for 5 min. Proteins were separated on 18% SDS-polyacrylamide gels and visualized using Coomassie stain. Cleavage of Ras isoforms and GTPases was quantified from scanned gels using NIH Image J 1.64. To identify the cleavage site, proteins separated by 18% SDS-polyacrylamide were transferred onto a polyvinylidene difluoride membrane. After Coomassie staining, processed bands were excised from the membrane and sequenced on an ABI 494 Procise Protein Sequencer (Applied Biosystem) using automated Edman degradation at the Tufts University Core Facility.

In-Vivo Cleavage Assay of Small GTPases.

DNA sequences coding for HRas, Rap1A, Rit2, RalA, Rheb2A, RhoB and Arf1 were amplified from plasmids for overexpression of GST-GTPases as described above68. Products were inserted into pEGFP-C3 (Clontech) using SmaI and the Gibson Assembly Cloning Kit (NEB). HEK 293T cells were transfected with the resulting plasmids as described above. After 24 h, cells were intoxicated with LFN proteins and cleavage detected using monoclonal GFP-HRP antibody (Miltenyi Biotec) as described above. The amount of cleaved protein as a percent of total GFP protein was quantified from scanned gels using NIH Image J 1.64 and data were normalized to the pixels detected in the absence of intoxication.

Bacterial Challenge of HeLa Cells.

V. vulnificus rifampicin-resistant isolates of strains CMCP6, M06-24/O and CMCP6DrtxA1 (ref 19) were grown at 30° C. in Luria-Bertani medium with 50 μg ml⁻¹ rifampicin. Overnight cultures were diluted 1:500 and grown at 30° C. with shaking until the OD₆₀₀ reached 0.55-0.6. Bacteria from 1 ml were pelleted at 1,800 g for 4 min, washed once in PBS and then resuspended in 1 ml PBS. Media were exchanged over 5×10⁴ HeLa cells previously seeded in 12-well plates overnight for antibiotic-free media. V. vulnificus in PBS (multiplicity of infection=100) or an equal volume of buffer was added to media over cells and plates were centrifuged at 25° C. for 5 min at 500 g. After 60 min, cells were photographed as described above, to assess rounding before collection of lysate and western blotting of proteins in 15 ml of lysate as described above. In a separate set of experiments, cells in phenol red-free DMEM with 10% fetal bovine serum but no antibiotics were incubated up to 4 h. At 1-h intervals, 50 ml of supernatant were sampled and assayed for release of lactate dehydrogenase using the Cytotox 96 Non-Radioactive Cytotoxicity Assay (Promega), according to the manufacturer's protocol. Percent cell lysis was calculated as the lactate dehydrogenase release in the sample divided by a positive control lysed with 0.1% Triton X-100.

### REFERENCES

1. Santos, E. et al. Malignant activation of a K-ras oncogene in lung carcinoma but not in normal tissue of the same patient. Science 223, 661-664 (1984).
2. Malumbres, M. & Barbacid, M. RAS oncogenes: the first 30 years. Nat. Rev. Cancer 3, 459-465 (2003).
3. Cox, A. D. & Der, C. J. Ras history: the saga continues. Small GTPases 1, 2-27 (2010).
4. Young, A., Lou, D. & McCormick, F. Oncogenic and wild-type Ras play divergent roles in the regulation of mitogen-activated protein kinase signaling. Cancer Discov. 3, 112-123 (2013).
5. Vogelstein, B. et al. Cancer genome landscapes. Science 339, 1546-1558 (2013).
6. Prior, I. A., Lewis, P. D. & Mattos, C. A comprehensive survey of Ras mutations in cancer. Cancer Res. 72, 2457-2467 (2012).
7. Cox, A. D., Fesik, S. W., Kimmelman, A. C., Luo, J. & Der, C. J. Drugging the undruggable RAS: Mission Possible? Nat. Rev. Drug Discov. 13, 828-851 (2014).
8. Burns, M. C. et al. Approach for targeting Ras with small molecules that activate SOS-mediated nucleotide exchange. Proc. Natl Acad. Sci. USA 111, 3401-3406 (2014).
9. Khvalevsky, E. Z. et al. Mutant KRAS is a druggable target for pancreatic cancer. Proc. Natl Acad. Sci. USA 110, 20723-20728 (2013).
10. Ostrem, J. M., Peters, U., Sos, M. L., Wells, J. A. & Shokat, K. M. K-Ras(G12C) inhibitors allosterically control GTP affinity and effector interactions. Nature 503, 548-551 (2013).
11. Russo, M., Di Nicolantonio, F. & Bardelli, A. Climbing RAS, the everest of oncogenes. Cancer Discov. 4, 19-21 (2014).
12. Shima, F. et al. In silico discovery of small-molecule Ras inhibitors that display antitumor activity by blocking the Ras-effector interaction. Proc. Natl Acad. Sci. USA 110, 8182-8187 (2013).
13. David, M. D., Cochrane, C. L., Duncan, S. K. & Schrader, J. W. Pure lipopolysaccharide or synthetic lipid a induces activation of p21Ras in primary macrophages through a pathway dependent on Src family kinases and PI3K. J. Immunol. 175, 8236-8241 (2005).
14. Zeiser, J., Gerhard, R., Just, I. & Pich, A. Substrate specificity of clostridia! glucosylating toxins and their function on colonocytes analyzed by proteomics techniques. J. Proteome Res. 12, 1604-1618 (2013).
15. Aktories, K. & Schmidt, G. in Ras Superfamily Small G Proteins: Biology and Mechanisms 1. (ed. Wittinghofer, A.) 65-97 (Springer-Verlag Wein, 2014).
16. Simon, N. C., Aktories, K. & Barbieri, J. T. Novel bacterial ADP-ribosylating toxins: structure and function. Nat. Rev. Microbiol. 12, 599-611 (2014).
17. Satchell, K. J. Structure and function of MARTX toxins and other large repetitive RTX proteins. Annu. Rev. Microbiol. 65, 71-90 (2011).
18. Egerer, M. & Satchell, K. J. Inositol hexakisphosphate-induced autoprocessing of large bacterial protein toxins PLoS Pathog. 6, e1000942 (2010).
19. Kwak, J. S., Jeong, H. G. & Satchell, K. J. Vibrio vulnificus rtxA1 gene recombination generates toxin variants with altered potency during intestinal infection. Proc. Natl Acad. Sci. USA 108, 1645-1650 (2011).
20. Antic, I., Biancucci, M. & Satchell, K. J. Cytotoxicity of the Vibrio vulnificus MARTX toxin effector DUF5 is linked to the C2A subdomain. Proteins 82, 2643-2656 (2014).
21. Geissler, B., Tungekar, R. & Satchell, K. J. Identification of a conserved membrane localization domain within numerous large bacterial protein toxins Proc. Natl Acad. Sci. USA 107, 5581-5586 (2010).
22. Brothers, M. C. et al. Backbone and side-chain assignments of an effector membrane localization domain from Vibrio vulnificus MARTX toxin. Biomol. NMR Assign. 8, 225-228 (2014).
23. Cherry, J. M. et al. Saccharomyces genome database: the genomics resource of budding yeast. Nucleic Acid Res. 40, D700-D705 (2012).
24. Mendoza, M. C., Er, E. E. & Blenis, J. The Ras-ERK and PI3K-mTOR pathways: cross-talk and compensation. Trends Biol. Sci. 36, 320-328 (2011).
25. Eisenberg, S. et al. The role of palmitoylation in regulating Ras localization and function. Biochem. Soc. Trans. 41, 79-83 (2013).
26. Hamer, P. J. et al. Production and characterization of anti-RAS p21 monoclonal antibodies. Hybridoma 9, 573-587 (1990).
27. Buhrman, G., Holzapfel, G., Fetics, S. & Mattos, C. Allosteric modulation of Ras positions Q61 for a direct role in catalysis. Proc. Natl Acad. Sci. USA 107, 4931-4936 (2010).
28. Rubinfeld, H. & Seger, R. The ERK cascade: a prototype of MAPK signaling Mol. Biotechnol. 31, 151-174 (2005).
29. Seger, R. & Krebs, E. G. The MAPK signaling cascade. FASEB J. 9, 726-735 (1995).
30. Spoerner, M., Herrmann, C., Vetter, I. R., Kalbitzer, H. R. & Wittinghofer, A. Dynamic properties of the Ras switch I region and its importance for binding to effectors. Proc. Natl Acad. Sci. USA 98, 4944-4949 (2001).
31. Caron, E., Self, A. J. & Hall, A. The GTPase Rap1 controls functional activation of macrophage integrin alphaMbeta2 by LPS and other inflammatory mediators. Curr. Biol. 10, 974-978 (2000).
32. Moon, E. Y. & Pyo, S. Lipopolysaccharide stimulates Epac1-mediated Rap1/NFkappaB pathway in Raw 264.7 murine macrophages. Immunol. Lett. 110, 121-125 (2007).
33. Roig, F. J., Gonzalez-Candelas, F. & Amaro, C. Domain organization and evolution of multifunctional autoprocessing repeats-in-toxin (MARTX) toxin in Vibrio vulnificus. Appl. Environ. Microbiol. 77, 657-668 (2011).
34. Liu, M., Alice, A. F., Naka, H. & Crosa, J. H. The HlyU protein is a positive regulator of rtxA1, a gene responsible for cytotoxicity and virulence in the human pathogen Vibrio vulnificus. Infect. Immun. 75, 3282-3289 (2007).
35. Lee, J. H. et al. Identification and characterization of the Vibrio vulnificus rtxA essential for cytotoxicity in vitro and virulence in mice. J. Microbiol. 45, 146-152 (2007).
36. Davies, H. et al. Mutations of the BRAF gene in human cancer. Nature 417, 949-954 (2002).
37. Okada, T. et al. The strength of interaction at the Raf cysteine-rich domain is a critical determinant of response of Raf to Ras family small GTPases. Mol. Cell. Biol. 19, 6057-6064 (1999).
38. Raaijmakers, J. H. & Bos, J. L. Specificity in Ras and Rap signaling. J. Biol. Chem. 284, 10995-10999 (2009).
39. Coburn, J. & Gill, D. M. ADP-ribosylation of p21ras and related proteins by Pseudomonas aeruginosa exoenzyme S. Infect. Immun. 59, 4259-4262 (1991).
40. Ganesan, A. K. et al. Pseudomonas aeruginosa exoenzyme S, a double ADPribosyltransferase, resembles vertebrate mono-ADP-ribosyltransferases. J. Biol. Chem. 274, 9503-9508 (1999).
41. Riese, M. J., Wittinghofer, A. & Barbieri, J. T. ADP ribosylation of Arg41 of Rap by ExoS inhibits the ability of Rap to interact with its guanine nucleotide exchange factor, C3G. Biochemistry 40, 3289-3294 (2001).
42. Rangel, S. M., Logan, L. K. & Hauser, A. R. The ADP-ribosyltransferase domain of the effector protein ExoS inhibits phagocytosis of Pseudomonas aeruginosa during pneumonia. mBio 5, e01080-e01014 (2014).
43. Fraylick, J. E., Rucks, E. A., Greene, D. M., Vincent, T. S. & Olson, J. C. Eukaryotic cell determination of ExoS ADP-ribosyltransferase substrate specificity. Biochem. Biophys. Res. Commun. 291, 91-100 (2002).
44. Maresso, A. W., Deng, Q., Pereckas, M. S., Wakim, B. T. & Barbieri, J. T. Pseudomonas aeruginosa ExoS ADP-ribosyltransferase inhibits ERM phosphorylation. Cell Microbiol. 9, 97-105 (2007).
45. Coburn, J., Dillon, S. T., Iglewski, B. H. & Gill, D. M. Exoenzyme S of Pseudomonas aeruginosa ADP-ribosylates the intermediate filament protein vimentin. Infect. Immun. 57, 996-998 (1989).
46. Just, I., Selzer, J., Hofmann, F., Green, G. A. & Aktories, K. Inactivation of Ras by Clostridium sordellii lethal toxin-catalyzed glucosylation. J. Biol. Chem. 271, 10149-10153 (1996).
47. Popoff, M. R. et al. Ras, Rap, and Rac small GTP-binding proteins are targets for Clostridium sordellii lethal toxin glucosylation. J. Biol. Chem. 271, 10217-10224 (1996).
48. Dreger, S. C. et al. Killing of rat basophilic leukemia cells by lethal toxin from Clostridium sordellii: critical role of phosphatidylinositide 3′-OH kinase/Akt signaling Biochemistry 48, 1785-1792 (2009).
49. Genth, H. & Just, I. Functional implications of lethal toxin-catalysed glucosylation of (H/K/N)Ras and Rac1 in Clostridium sordellii-associated disease. Eur. J. Cell Biol. 90, 959-965 (2011).
50. Guttenberg, G. et al. Molecular characteristics of Clostridium perfringens TpeL toxin and consequences of mono-O-GlcNAcylation of Ras in living cells. J. Biol. Chem. 287, 24929-24940 (2012).
51. Pauillac, S. et al. Characterization of the enzymatic activity of Clostridium perfringens TpeL. Toxicon 75, 136-143 (2013).
52. Just, I. et al. Glucosylation of Rho proteins by Clostridium difficile toxin B. Nature 375, 500-503 (1995).
53. Jank, T., Pack, U., Giesemann, T., Schmidt, G. & Aktories, K. Exchange of a single amino acid switches the substrate properties of RhoA and RhoD toward glucosylating and transglutaminating toxins J Biol. Chem. 281, 19527-19535 (2006).
54. Jank, T., Giesemann, T. & Aktories, K. Clostridium difficile glucosyltransferase toxin B-essential amino acids for substrate binding. J. Biol. Chem. 282, 35222-35231 (2007).
55. Lo, H. R. et al. RTX toxin enhances the survival of Vibrio vulnificus during infection by protecting the organism from phagocytosis. J. Infect. Dis. 203, 1866-1874 (2011).
56. Jeong, H. G. & Satchell, K. J. F. Additive function of Vibrio vulnificus MARTX_{Vv} and _{Vv}hA cytolysins promotes rapid growth and epithelial tissue necrosis during intestinal infection. PLoS Pathog. 8, e1002581 (2012).
57. Kashimoto, T. et al. Vibrio vulnificus detected in the spleen leads to fatal outcome in a mouse oral infection model. FEMS Microbiol. Lett. 362, fnv005 (2015).
58. Thiaville, P. C. et al. Genotype is correlated with but does not predict virulence of Vibrio vulnificus biotype 1 in subcutaneously inoculated, iron dextrantreated mice. Infect. Immun. 79, 1194-1207 (2011).
59. Lemichez, E. & Aktories, K. Hijacking of Rho GTPases during bacterial infection. Exp. Cell. Res. 319, 2329-2336 (2013).
60. Mechaly, A., McCluskey, A. J. & Collier, R. J. Changing the receptor specificity of anthrax toxin. mBio 3, e00088-12 (2012).
61. McCluskey, A. J., Olive, A. J., Starnbach, M. N. & Collier, R. J. Targeting HER2-positive cancer cells with receptor-redirected anthrax protective antigen. Mol. Oncol. 7, 440-451 (2013).
62. Antignani, A. & Fitzgerald, D. Immunotoxins: the role of the toxin. Toxins 5, 1486-1502 (2013).
63. Van Dessel, N. et al. Potent and tumor specific: arming bacteria with therapeutic proteins. Ther. Deliv. 6, 385-399 (2015).
64. Bell, J. & McFadden, G. Viruses for tumor therapy. Cell. Host Microbe 15, 260-265 (2014).
65. Willhite, D. C. & Blanke, S. R. Soluble expression and one-step purification of recombinant Bacillus anthracis protective antigen. Protein Peptide Lett. 5, 273-278 (1998).
66. Puck, T. T. & Marcus, P. I. A rapid method for viable cell titration and clone production with HeLa cells in tissue culture—the use of X-irradiated cells to supply conditioning factors. Proc. Natl Acad. Sci. USA 41, 432-437 (1955).
67. Stols, L. et al. A new vector for high-throughput, ligation-independent cloning encoding a tobacco etch virus protease cleavage site. Protein Expr. Purif. 25, 8-15 (2002).
68. Mattoo, S. et al. Comparative analysis of Histophilus somni immunoglobulinbinding protein A (IbpA) with other Fic domain-containing enzymes reveals differences in substrate and nucleotide specificities. J. Biol. Chem. 286, 32834-32842 (2011).
69. Pai, E. F. et al. Refined crystal-structure of the triphosphate conformation of H-Ras P21 at 1.35 a resolution—implications for the mechanism of GTP hydrolysis. EMBO J. 9, 2351-2359 (1990).

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

1. A method of treating a cell proliferative disease or disorder in a patient, the method comprising administering a therapeutic polypeptide comprising the amino acid sequence of a DUF5 protease or a portion of a DUF5 protease to the patient.

2. The method of claim 1, wherein the cell proliferative disease or disorder is associated with an activating mutation in a Ras protein.

3. The method of claim 1, wherein the Ras protein is selected from the group consisting of KRAS, HRAS, or NRAS.

4. The method of claim 2, wherein the activating mutation is present in codon 12, 13, or 61 of the Ras protein.

5. The method of claim 1, wherein the cell proliferative disease or disorder is a tumor of a primary tissue selected from the group consisting of adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus.

6. The method of claim 1, wherein the DUF5 protease is selected from the group consisting of Vibrio vulnificus DUF5 protease, Vibrio ordalii DUF5 protease, Vibrio cholerae DUF5 protease, Vibrio spendidus DUF5 protease, Moritella dasanensis DUF5 protease, Aeromonas salmonicida DUF5 protease, Aeromonas hydrophila DUF5 protease, Photorhabdus temperata DUF5 protease, Xenorhabdus nematophila DUF5 protease, Photorhabdus luminescens DUF5 protease, Photorhabdus asymbiotica DUF5 protease, Yersinia kristensenii DUF5 protease, and Pasteurella multocida DUF5 protease.

7. The method of claim 1, wherein the therapeutic polypeptide comprises the amino acid sequence of any of SEQ ID NOs:1, 3, 5, 7, 9, 11, 13, 15, 17, 19, 21, 23, 25, and 27 or an amino acid sequence having at least 50% sequence identity with the amino acid sequence of any of SEQ ID NOs:1, 3, 5, 7, 9, 11, 13, 15, 17, 19, 21, 23, 25, and 27 and the therapeutic polypeptide cleaves a Ras protein.

8. The method of claim 1, wherein the therapeutic polypeptide comprises the amino acid sequence of any of SEQ ID NOs:2, 4, 6, 8, 9, 10, 12, 14, 16, 18, 20, 22, 24, 26, and 28 or an amino acid sequence having at least 50% sequence identity with the amino acid sequence of any of SEQ ID NOs:2, 4, 6, 8, 9, 10, 12, 14, 16, 18, 20, 22, 24, 26, and 28 and the therapeutic polypeptide cleaves a Ras protein.

9. The method of claim 7, wherein the therapeutic polypeptide cleaves the Ras protein between a tyrosine at amino acid position 32 and an aspartic acid at amino acid position 33.

10. The method of claim 8, wherein the therapeutic polypeptide cleaves the Ras protein between a tyrosine at amino acid position 32 and an aspartic acid at amino acid position 33.

11. The method of claim 1, wherein the therapeutic polypeptide is formulated as a pharmaceutical composition for delivering the therapeutic polypeptide to proliferating cells.

12. The method of claim 11, wherein the therapeutic polypeptide comprises the DUF5 protease or the portion of the DUF5 protease fused or complexed with a carrier in the pharmaceutical composition that facilitates transport of the DUF5 protease or the portion of the DUF5 protease into the proliferating cells.

13. The method of claim 12, wherein the therapeutic polypeptide comprises the DUF5 protease or the portion of the DUF5 protease fused to anthrax toxin lethal factor N-terminus (LF_{N}).

14. The method of claim 13, wherein the therapeutic polypeptide is contacted with anthrax toxin protective antigen (PA) to form a complex that is delivered to the cytosol of proliferating cells.

15. A pharmaceutical composition comprising a DUF5 protease or the portion of the DUF5 protease fused or complexed with a carrier that facilitates transport of the DUF5 protease or the portion of the DUF5 protease into proliferating cells.

16. The pharmaceutical composition of claim 15, comprising a fusion protein comprising DUF5 protease or the portion of the DUF5 protease fused at its N-terminus to anthrax toxin lethal factor N-terminus (LF_{N}).

17. The pharmaceutical composition of claim 16 further comprising anthrax toxin protective antigen (PA) which forms a complex with the fusion protein and the complex is delivered to the cytosol of proliferating cells.

18. The pharmaceutical composition of claim 15, wherein the DUF5 protease is selected from the group consisting of Vibrio vulnificus DUF5 protease, Vibrio ordalii DUF5 protease, Vibrio cholerae DUF5 protease, Vibrio spendidus DUF5 protease, Moritella dasanensis DUF5 protease, Aeromonas salmonicida DUF5 protease, Aeromonas hydrophila DUF5 protease, Photorhabdus temperata DUF5 protease, Xenorhabdus nematophila DUF5 protease, Photorhabdus luminescens DUF5 protease, Photorhabdus asymbiotica DUF5 protease, Yersinia kristensenii DUF5 protease, and Pasteurella multocida DUF5 protease.

19. The pharmaceutical composition of claim 15, wherein the DUF5 protease or the portion of the DUF5 protease comprises the amino acid sequence of any of SEQ ID NOs:1, 3, 5, 7, 9, 11, 13, 15, 17, 19, 21, 23, 25, and 27 or an amino acid sequence having at least 50% sequence identity with the amino acid sequence of SEQ ID NO:1 and the DUF5 protease cleaves a Ras protein.

20. The pharmaceutical composition of claim 15, wherein the DUF5 protease or the portion of the DUF5 protease comprises the amino acid sequence of any of SEQ ID NOs:2, 4, 6, 8, 9, 10, 12, 14, 16, 18, 20, 22, 24, 26, and 28 or an amino acid sequence having at least 50% sequence identity with the amino acid sequence of any of SEQ ID NOs:2, 4, 6, 8, 9, 10, 12, 14, 16, 18, 20, 22, 24, 26, and 28 and the DUF5 protease or the portion of the DUF5 protease cleaves a Ras protein.
